(19) United States
(12) Patent Application Publication
(10) Pub. No.: US 2024/0122864 A1
(43) Pub. Date: Apr. 18, 2024

(54) PARTICLES ENCAPSULATING FUSION PROTEINS CONTAINING LINKED EPITOPES

(71) Applicant: COUR PHARMACEUTICALS DEVELOPMENT COMPANY INC., Skokie, IL (US)

(72) Inventor: Daniel R Getts, Northbrook, IL (US)

(21) Appl. No.: 18/333,103

(22) Filed: Jun. 12, 2023

Related U.S. Application Data
(63) Continuation of application No. 16/067,867, filed on Jul. 3, 2018, now abandoned.
(60) Provisional application No. 62/274,711, filed on Jan. 4, 2016.

(51) Int. Cl. A61K 9/50; A61K 39/00; A61P 35/00

(52) U.S. Cl. CPC A61K 9/5031; A61K 39/0007; A61K 39/0008; A61K 39/001186; A61K 39/001188; A61P 35/00; A61K 2039/55555; A61K 2039/572; A61K 2039/70

(57) ABSTRACT

The present invention provides compositions comprising biodegradable particles that encapsulate two or more epitopes linked together by one or more linkers that are susceptible to cleavage by specific proteases. The present invention further provides methods for inducing antigen-specific tolerance and protective immune responses and for the treatment inflammatory diseases, such as autoimmune diseases, allergies, cancers, or infectious diseases.

### CROSS-REFERENCE TO RELATED APPLICATIONS

This application claims priority to U.S. Provisional Application No. 62/274,711, filed Jan. 4, 2016, the content of which is incorporated herein by reference in its entirety.

### DESCRIPTION OF THE TEXT FILE SUBMITTED ELECTRONICALLY

The contexts of the text file submitted electronically herewith are incorporated herein by reference in their entirety: A computer-readable format copy of the Sequence Listing (file name: COUR-013_01WO_Seglist.txt, date recorded: Jan. 4, 2016, file size: 1.17 megabytes)

### BACKGROUND OF THE INVENTION

Inflammatory diseases and disorders are conditions in which an abnormal or otherwise deregulated inflammatory response contributes to the etiology or severity of disease and encompass a wide range of maladies including autoimmune diseases, allergies, cancers, and infections.

Conventional clinical strategies for general long-term immunosuppression in disorders associated with an undesired immune response are based on the long-term administration of broad acting immunosuppressive drugs, for example, signal 1 blockers such as cyclosporin A (CsA), FK506 (tacrolimus) and corticosteroids. However, these drugs often require high doses in order to achieve the desired efficacy and long-term use often results in toxic side-effects. Moreover, even in patients that tolerate these drugs, the requirement for life-long immunosuppressive drug therapy carries a significant risk of severe side effects, nephrotoxicity, and metabolic disorders. Further, general, non-specific immune suppression inhibits both pathological (e.g., autoreactive) and beneficial immune responses, such as those elicited against cancerous cells and infectious agents. As a result, patients undergoing immunosuppressive treatments are at increased risk for the development of various cancers and contraction of serious infections.

Similarly, cancer therapeutics generally result in broad, non-specific immune activation in attempts to eradicate cancerous cells. However, these broad activation strategies result in damage to healthy, non-cancerous or non-malignant tissue and even death. Therefore, there exists a need in the art for improved therapeutics that can effectively regulate immune responses in an antigen-specific manner. Such therapeutics would allow for the targeted treatment of inflammatory diseases, such as autoimmune diseases and cancer, thereby minimizing the negative side effects associated with broad, non-specific activation or inhibition of the immune system.

Methods of inducing antigen-specific tolerance have been developed, including cell coupling of an antigen or peptide. For example, in one method, peptide-induced, cell-coupled tolerance involves collection, separation, and treatment of peripheral blood cells with disease specific autoantigens and the coupling reagent ethylene carbodimide (ECDI) under sterile conditions. These peptide-coupled cells are subsequently re-infused into the donor/patient. This process is costly and must be conducted under closely monitored conditions by skilled practitioners and is limited in the number of centers that can conduct the procedure. Although the use of red blood cells as the donor cell type expands the potential source to include allogeneic donors, dramatically increases the supply of source cells, and potentially expands the number of suitable delivery centers to include any setting certified for blood transfusion, significant drawbacks remain including a limited supply of source cells and necessity for blood-type matching to minimize immune response to the donor cells.

Recently, peptide-coupled particles have been described which eliminate the requirement for a supply of source cells and circumvents the tissue-typing requirement of the prior approaches (See U.S. Patent Publication No. 2012-0076831, incorporated by reference herein in its entirety). Notwithstanding, the use of antigens coupled to the outside of particles is associated with increased anaphylaxis and has significant chemistry, manufacturing and control issues. However, when an antigen is encapsulated within the particle, these adverse events can be avoided. Surprisingly, the size and the charge can be altered to control the phenotype of the immune response elicited, inducing either enhanced tolerogenic or regulatory responses (e.g., in the context of autoimmunity) or enhanced protective immune responses (e.g., in the context of cancer) to specific antigens.

While particles encapsulating a single epitope or protein have been created, many of disease pathologies are related to multiple proteins. Even further, a single disease may have several immunogenic epitopes within each antigen. For example, multiple sclerosis (MS) is thought to involve inflammatory reactions to multiple sites of several different autologous proteins, including at least proteolipid protein (PLP), myelin oligodendrocyte glycoprotein (MOG), and myelin basic protein (MBP). The primary target antigens, however, are not known for certain. What is more, the target antigens differ between patients and the T cell reactivity to different antigens changes over time in a process known as epitope spreading. Thus in order to ensure coverage of all possible antigenic epitopes that a particular MS patient may be reactive towards at any given point, encapsulation of the entire proteins would be necessary. Given the size and physical properties of these large proteins, however, encapsulation is exceedingly difficult. As such, particles encapsulating multiple epitopes associated with a particular disease would likely increase the therapeutic efficacy of such particles. However, the properties of different proteins, such as solubility or isoelectric point, increase the complexity of manufacturing particles encapsulating more than one protein or epitope, and often result in variable and difficult to control encapsulation efficiencies.

Further, methods inducing antigen-specific immune activation, for example against tumor antigens, have also been developed, including the use of multiple epitope constructs expressing MAGE3 and HPV in the treatment of squamous cell carcinoma of the head and neck (See U.S. Pat. Nos. 8,263,560 and 7,842,480). Additionally, a multi-epitope system using RNA-lipoplexes that utilizes individual RNA vectors encoding four tumor antigens (NY-ESO-1, MAGE-A3, tyrosinase, and TPTE) encapsulated into liposomes (RNA-LPX) has also been described (See Kranz et al., Nature, V. 534, pp. 396-401, 2016). Administration of these RNA-LPXs induced systemic IFNα responses and amplified T cell responses against the encoded antigens. However, this system does not overcome the challenges associated with encapsulating multiple, independent components into a single particle. Variations in the encapsulation efficiency may result in a disproportionate incorporation of one component over the other. Further, the use of nucleic acid-based vaccines requires the use of endogenous transcription and translation pathways. Each of these factors increases the variability of the system, altering the relative expression of the encoded proteins and decreasing the therapeutic efficacy of the composition. As such, there is a need in the art for compositions and methods that allow for the incorporation of multiple disease epitopes into a single therapeutic composition for use in the treatment of inflammatory diseases, particularly those diseases where multiple epitopes or proteins are involved in pathogenesis.

### SUMMARY OF THE INVENTION

The present invention provides biodegradable particles that encapsulate two or more epitopes linked together by one or more linkers. The linkers are amino acid sequences that are susceptible to cleavage by specific proteases, allowing for control over antigen presentation by major histocompatibility (MHC)-I or MHC-II and further enhancing control over the resultant immune response. Linking the epitopes into a single protein allows for a particle that can induce tolerance to multiple epitopes that is capable of delivering the epitopes at a controlled ratio to each other. Such particles are useful for ameliorating inflammatory diseases, such as autoimmune diseases or allergies, associated with more than one epitope. Incorporation of immune modulators and agonists, such as TLR agonists also for the use of such particles the treatment of cancers.

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the biodegradable particle has a zeta potential of about −100 mV to about 0 mV. In further embodiments, the biodegradable particle has a zeta potential of about −50 mV to about −40 mV. In further embodiments, the biodegradable particle has a zeta potential of about −75 mV to about −50 mV. In further embodiments, the biodegradable particle has a zeta potential of about −50 mV.

In some embodiments, the biodegradable particle comprises poly(lactide-co-glycolide) (PLG). In further embodiments, the biodegradable particle comprises PLG with a copolymer ratio of about 50:50 of polylactic acid:polyglycolic acid. In some embodiments, the surface of the biodegradable particle is carboxylated. In further embodiments, the carboxylation is achieved by using poly(ethylene-maleic anhydride) (PEMA), poly-acrylic acid, or sodium cholate.

In some embodiments, the biodegradable particle has a diameter of between about 0.1 μm to about 10 μm. In some embodiments, the biodegradable particle has a diameter of between about 0.3 μm to about 5 μm. In some embodiments, the biodegradable particle has a diameter of between about 0.5 μm to about 3 μm. In some embodiments, the biodegradable particle has a diameter of between about 0.5 μm to about 1 μm. In some embodiments, the biodegradable particle has a diameter of about 0.5 μm. In some embodiments, the biodegradable particle has a diameter of about 0.6 μm.

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, and wherein said biodegradable particle has a negative zeta potential. In some embodiments, the linker comprises an amino acid sequence susceptible to specific cleavage by a protease located in the phagolysosome of a cell or a site susceptible to a specific cleavage by a protease located in the cytosol of the cell. In some embodiments, the linker comprises an amino acid sequence susceptible to specific cleavage by a protease located in the phagolysosome of a cell and a site susceptible to a specific cleavage by a protease located in the cytosol of the cell.

In some embodiments, the site susceptible to specific cleavage by a protease located in the phagolysosome is susceptible to cleavage by a furin or cathepsin protease. In further embodiments, the site susceptible to specific cleavage by a protease located in the phagolysosome is susceptible to cleavage by a furin protease. In further embodiments, the site susceptible to specific cleavage by a protease located in the phagolysosome is susceptible to cleavage by a cathepsin protease. In still further embodiments, the site susceptible to specific cleavage by a protease located in the phagolysosome is one or more of cathepsin A, cathepsin B, cathepsin C, cathepsin D, cathepsin E, cathepsin F, cathepsin G, cathepsin H, cathepsin K, cathepsin L, cathepsin O, cathepsin W, or cathepsin Z. In still further embodiments, the site susceptible to specific cleavage by a protease located in the phagolysosome is cathepsin L.

In further embodiments, the site susceptible to specific cleavage by a protease located in the cytosol is susceptible to cleavage by a furin or cathepsin protease. In further embodiments, the site susceptible to specific cleavage by a protease located in the cytosol is susceptible to cleavage by cathepsin S. In further embodiments, the amino acid sequence of the linker comprises a site susceptible to specific cleavage by cathepsin L and a site susceptible to a specific cleavage by cathepsin S. In still further embodiments, the amino acid sequence of the linker is Gly-Ala-Val-Val-Arg-Gly-Ala (SEQ ID NO: 5141).

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the two or more antigenic epitopes comprise autoimmune antigens, antigens expressed on a tissue to be transplanted into a subject, antigens derived from an enzyme for enzyme replacement therapy, or antigens derived from an allergen. In further embodiments, the two or more antigenic epitopes each comprise at least a portion of a protein, wherein said portions are from the same protein. In further embodiments, the two or more antigenic epitopes each comprise at least a portion of a protein, wherein said portions are from different proteins. In some embodiments, the different proteins are associated with the same autoimmune disorder, the same tissue to be transplanted into a subject, or the same allergen.

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the two or more antigenic epitopes each comprise at least a portion of a protein selected from the group consisting of: myelin basic protein, acetylcholine receptor, endogenous antigen, myelin oligodendrocyte glycoprotein, pancreatic beta-cell antigen, insulin, glutamic acid decarboxylase (GAD), collagen type 11, human cartilage gp39, fp130-RAPS, proteolipid protein, fibrillarin, small nucleolar protein, thyroid stimulating factor receptor, histones, glycoprotein gp70, pyruvate dehydrogenase dihydrolipoamide acetyltransferase (PCD-E2), hair follicle antigen, A-gliadin, gliadin, insulin, proinsulin, islet specific glucose-6-phosphatase catalytic subunit-related protein (IGRP), human tropomyosin isoform 5, Bahia grass pollen (BaGP), peach allergen Pru p 3, alpha s 1-Caein Milk allergen, Apigl celery allergen, Berel Brazil nut allergen, B-Lactoglobulin Milk allergen, Bovine serum albumin, Cor a 1.04 Hazelnut allergen, myelin associated glycoprotein, aquaporin α3 chain of type IV collagen, Ovalbumin Egg allergen, Advate, antihemophilic factor, Kogenate, Eloctate, recombinant factor VIII Fc fusion protein, Refacto, Novo VIIa, recombinant factor VII, eptacog alfa, Helixate, Monanine, Coagulation Factor IX, Wilate, Ceredase, Alglucerase, Cerezyme, Imiglucerase, Elelso, taliglucerase alfa, Fabrazyme, Agalsidase beta, Aldurazyme, -I-iduronidase, Myozyme, Acid-glucosidase, Elaprase, iduronate-2-sulfatase, Naglazyme arylsufatase B, or N-acetylgalactosamine-4-sulfatase.

In some embodiments, the two or more antigenic epitopes are selected from the group consisting of SEQ ID NOS: 2-1294. In further embodiments, the two or more antigenic epitopes are selected from the group consisting of SEQ ID NOs: 1295-1724; SEQ ID NOs: 1726-1766; SEQ ID NOs: 4986-5140; and discontinuous epitopes derived from SEQ ID NO: 1725.

In some embodiments, the two or more antigenic epitopes are selected from the group consisting of SEQ ID NOs: 1767-1840; SEQ ID NOs: 1842-1962; SEQ ID NOs: 1964-2027; SEQ ID NOs: 2029-2073; SEQ ID NOs: 2075-2113; SEQ ID NOs: 2115-2197; SEQ ID NOs: 2199-2248; SEQ ID NOs: 2250-2259; SEQ ID NOs: 2261-2420; SEQ ID NOs: 2422-2486; SEQ ID NOs: 2489-2505; and discontinuous epitopes derived from SEQ ID NOs: 1841, 1963, 2028, 2074, 2114, 2198, 2260, 2249, 2421, 2487, and 2488.

In some embodiments, the two or more antigenic epitopes are selected from the group consisting of SEQ ID NOs: 2506-3260; SEQ ID NOs: 3262-3693; and discontinuous epitopes derived from 3261. In some embodiments, the two or more antigenic epitopes are selected from the group consisting of SEQ ID NOs: 3694-3857; SEQ ID NOs: 3860-4565; and discontinuous epitopes derived from 3857, 3858, and 3859. In some embodiments, the two or more antigenic epitopes are selected from the group consisting of SEQ ID NOs: 4566-4576; SEQ ID NOs: 4578-4610; SEQ ID NOs: 4612-4613; and SEQ ID NOs: 5018-5039; and discontinuous epitopes derived from 4357, 4577, and 4611.

In some embodiments, the two or more antigenic epitopes are selected from the group consisting of SEQ ID NOs: 4614-4653. In some embodiments, the two or more antigenic epitopes are selected from the group consisting of SEQ ID NOs: 4654-4694; SEQ ID NOs: 4696-4894; SEQ ID NOs: 4896-4901; and discontinuous epitopes derived from 4695 and 4895. In some embodiments, the two or more antigenic epitopes are selected from the group consisting of SEQ ID NOs: 4902-4906. In some embodiments, the two or more antigenic epitopes are selected from the group consisting of SEQ ID NOs: 4907-4914. In some embodiments, the two or more antigenic epitopes are selected from the group consisting of: SEQ ID NOs: 4915-4917. In some embodiments, the two or more antigenic epitopes are selected from the group consisting of SEQ ID NOs: 4918-4941. In some embodiments, the two or more antigenic epitopes are selected from the group consisting of SEQ ID NOs: 4942-4952. In some embodiments, the two or more antigenic epitopes are selected from the group consisting of SEQ ID NOs: 4953-4963. In some embodiments, the two or more antigenic epitopes are selected from the group consisting of SEQ ID NOs: 4964-4974.

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the two or more antigenic epitopes are derived from a therapeutic antibody, antigen-binding fragment, or Fc fragment thereof. In some embodiments, the antibody or antigen-binding fragment thereof is a monoclonal antibody, a humanized monoclonal antibody, a human monoclonal antibody, a chimeric antibody, a single chain antibody, fragment antigen binding region (Fab), a single chain variable fragment (scFv), small modular immunopharmaceutical (SMIP), or a single antigen-binding domain.

In further embodiments, the antibody or antigen-binding fragment thereof binds to α4β1 integrin, Bacillus anthracis, B-L gamma S, C5, CD3, CD11a, CD20, CD25, CD30, CD33, CD52, CD59, CTLA4, EGFR, GD2, GPIIb, IIIa, HER2, IgE, IL-1β, IL-5, IL12/23, PCSK9, PD1, RANK, RSV F protein, TNFα, or VEGF-A. In further embodiments, the antibody or antigen-binding fragment thereof is Abciximab, Adalimumab, Adotrastuzumab emtansine, Alemtuzumab, Basiliximab, Bevacizumab, Belimumab, Blinatumomab, Brentuximab Vedotin, Canakinumab, Catumaxomab, Cetuximab, Certolizumab pegol, Daclizumab, Denosumab, Dinutuximab, Eculizumab, Efalizumab, Evolocumab, Gemtuzumab ozogamicin, Golimumab, Ibritumomab tiuxetan, Ipilimumab, Infliximab, Motavizumab, Muromonab, Natalizumab, Nivolumab, Obinutuzumab, Ofatumumab, Omalizumab, Panitumumab, Palivizumab, Pembrolizumab, Pertuzumab, Ramucirumab, Ranibizumab, Raxibacumab, Rituximab, Secukinumab, Siltuximab, Trastuzumab, Tocilizumab, Tositumomab-I-131, Ustekinumab, or Vedolizumab.

In some aspect of the invention, the two or more antigenic epitopes are derived from a variant of a therapeutic antibody or antigen-binding fragment thereof that lacks functional complementarity determining regions (CDRs). In further aspects, the variant of the antibody or antigen-binding fragment thereof that lacks functional CDRs is a monoclonal antibody, a humanized monoclonal antibody, a human monoclonal antibody, a chimeric antibody, a single chain antibody, fragment antigen binding region (Fab), a single chain variable fragment (scFv), small modular immunopharmaceutical (SMIP), or a single antigen-binding domain.

Some aspects of the present invention provide for a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, one of said one or more fusion proteins comprises the antigenic epitopes MOG₁₋₂₀, MBP₁₃₋₃₂, MOG₃₅₋₅₅, MBP₁₄₆₋₁₇₀, PLP₁₃₉₋₁₅₄, MBP₁₁₁₋₁₂₉, and/or MBP₈₃₋₉₉. In further aspects, one of said one or more fusion proteins comprises the antigenic epitopes SEQ ID NO:1350, SEQ ID NO: 4986, and SEQ ID NO: 4987.

Some aspects of the present invention provide for a pharmaceutical composition comprising a biodegradable particle described herein. In further aspects, the pharmaceutical composition comprises a pharmaceutically acceptable carrier. In further aspects, the pharmaceutical composition comprises pharmaceutically acceptable excipients.

Some aspects of the present invention provide for methods of inducing antigen-specific tolerance in a subject comprising administering an effective amount of a biodegradable particle described herein. In some aspects, the method of inducing antigen-specific tolerance in a subject comprises administering to the subject an effective amount of a biodegradable particle comprising one or more fusion proteins encapsulated therein, wherein each one of said one or more fusion proteins comprises two or more antigenic epitopes, wherein said two or more antigenic epitopes are separated by a linker, wherein said linker comprises an amino acid sequence susceptible to specific cleavage, and wherein said biodegradable particle has a negative zeta potential. In some embodiments, the effective amount of the biodegradable particle is administered to the subject orally, intravenously, sublingually, buccally, enterically, topically, rectally, subcutaneously, nasally, intraosseously (i.e., intraosseous infusion), intraperitoneally, intrathecally, transdermally, or transmucosally. In further embodiments, the effective amount of the biodegradable particle is administered to the subject intravenously or subcutaneously. In further embodiments, the effective amount of the biodegradable particle is administered to the subject intravenously. In further embodiments, the effective amount of the biodegradable particle is administered to the subject subcutaneously.

In some embodiments, an effective amount of a biodegradable particle described herein is administered to the subject to treat or prevent a disease or condition. In some embodiments, the disease or condition is selected from the group consisting of: an autoimmune disease, a lysosomal storage disease, an enzyme deficiency, inflammatory disease, an allergy, transplantation rejection, and a hyper-immune response. In further embodiments, the disease or condition is selected from the group consisting of multiple sclerosis, type 1 diabetes, asthma, a food allergy, an environmental allergy, Celiac disease, inflammatory bowel disease, including Crohn's disease and ulcerative colitis, a mucopolysaccharide storage disorder, gangliosidosis, alkaline hypophosphatasia, cholesterol ester storage disease, hyperuricemia, growth hormone deficiency, renal anemia Hemophilia, Hemophilia A, Hemophilia B, von Willebrand disease, Gaucher's Disease, Fabry's Disease, Hurler's Disease, Pompe's Disease, Hunter's Disease, Maroteaux-Lary Disease and a condition caused by the antigen in the subject to produce an overreaction to the antigen.

In some embodiments, the disease or condition is multiple sclerosis, wherein each one of said one or more fusion proteins comprises two or more antigenic epitopes selected from the group consisting of SEQ ID NOS: 2-1294.

In some embodiments, the disease or condition is Celiac disease, wherein each one of said one or more fusion proteins comprises two or more antigenic epitopes selected from the group consisting of SEQ ID NOs: 1295-1724; SEQ ID NOs: 1726-1766; SEQ ID NOs: 4986-5140; and discontinuous epitopes derived from SEQ ID NO: 1725.

In some embodiments, the disease or condition is Type I Diabetes, wherein each one of said one or more fusion proteins comprises two or more antigenic epitopes selected from the group consisting of SEQ ID NOs: 1767-1840; SEQ ID NOs: 1842-1962; SEQ ID NOs: 1964-2027; SEQ ID NOs: 2029-2073; SEQ ID NOs: 2075-2113; SEQ ID NOs: 2115-2197; SEQ ID NOs: 2199-2248; SEQ ID NOs: 2250-2259; SEQ ID NOs: 2261-2420; SEQ ID NOs: 2422-2486; SEQ ID NOs: 2489-2505; and discontinuous epitopes derived from SEQ ID NOs: 1841, 1963, 2028, 2074, 2114, 2198, 2260, 2249, 2421, 2487, and 2488.

In some embodiments, the disease or condition is rheumatoid arthritis, wherein each one of said one or more fusion proteins comprises two or more antigenic epitopes selected from the group consisting of SEQ ID NOs 2506-3260; SEQ ID NOs: 3262-3693; and discontinuous epitopes derived from 3261.

In some embodiments, the disease or condition is systemic lupus, wherein each one of said one or more fusion proteins comprises two or more antigenic epitopes selected from the group consisting of SEQ ID NOs 3694-3857; SEQ ID NOs: 3860-4565; and discontinuous epitopes derived from 3857, 3858, and 3859.

In some embodiments, the disease or condition is Good Pasture's syndrome, wherein each one of said one or more fusion proteins comprises two or more antigenic epitopes selected from the group consisting of SEQ ID NOs: 4566-4576; SEQ ID NOs: 4578-4610; SEQ ID NOs: 4612-4613; and SEQ ID NOs: 5018-5039; and discontinuous epitopes derived from 4357, 4577, and 4611.

In some embodiments, the disease or condition is uveitis, wherein each one of said one or more fusion proteins comprises two or more antigenic epitopes selected from the group consisting of SEQ ID NOs: 4614-4653.

In some embodiments, the disease or condition is thyroiditis, wherein each one of said one or more fusion proteins comprises two or more antigenic epitopes selected from the group consisting of SEQ ID NOs: 4654-4694; SEQ ID NOs: 4696-4894; SEQ ID NOs: 4896-4901; and discontinuous epitopes derived from 4695 and 4895.

In some embodiments, the disease or condition is myositis, wherein each one of said one or more fusion proteins comprises two or more antigenic epitopes selected from the group consisting of SEQ ID NOs: 4902-4906.

In some embodiments, the disease or condition is vasculitis, wherein each one of said one or more fusion proteins comprises two or more antigenic epitopes selected from the group consisting of SEQ ID NOs: 4907-4914.

In some embodiments, the disease or condition is pancreatitis, wherein each one of said one or more fusion proteins comprises two or more antigenic epitopes selected from the group consisting of SEQ ID NOs: 4915-4917.

In some embodiments, the disease or condition is Crohn's disease, wherein each one of said one or more fusion proteins comprises two or more antigenic epitopes selected from the group consisting of SEQ ID NOs: 4918-4941.

In some embodiments, the disease or condition is ulcerative colitis, wherein each one of said one or more fusion proteins comprises two or more antigenic epitopes selected from the group consisting of SEQ ID NOs: 4942-4952.

In some embodiments, the disease or condition is psoriasis, wherein each one of said one or more fusion proteins comprises two or more antigenic epitopes selected from the group consisting of SEQ ID NOs: 4953-4963.

In some embodiments, the disease or condition is reactive arthritis, wherein each one of said one or more fusion proteins comprises two or more antigenic epitopes selected from the group consisting of SEQ ID NOs: 4964-4974.

Some aspects of the present invention provide methods for decreasing inhibitory neutrophil accumulation in a subject comprising administering to the subject an effective amount of a biodegradable particle described herein. In some embodiments, the subject has cancer. In some embodiments, the two or more antigenic epitopes each comprise at least a portion of a protein selected from the group consisting of CD19, CD20, BCMA, CD22, CLL1, CD33, CEA, CD123, CS1, EGFR, PSMA, EphA2, MCSP, ADAM17, PSCA, TPTE, HPU16, immature laminin receptor, TAG-72, HPV E6, HPV E7, BING-4, Calcium-activated chloride channel 2, cyclin B₁, 9D7, Ep-CAM, EphA3, Her2/neu, telomerase, mesothelin, SAP-1, survivin, proteins of the BAGE family, proteins of the CAGE family, proteins of the GAGE family, proteins of the MAGE family (e.g., MAGE-A3), proteins of the SAGE family, proteins of the RAGE family, CT9, CT10, NY-ESO1/LAGE-1, PRAME, SSX-2, Melan-A/MART-1, Cp100/pmel17, tyrosinase, TRP-1/TRP-2, P.polypeptide, MC1R, prostate-specific antigen, β-catenin, BRCA1/2, CDK4, CML66, fibronectin, MART-2, p53, Ras, TGF-βRII, and MUC1.

Some aspects of the present invention provide methods for increasing tissue regeneration in a subject comprising administering to the subject an effective amount of a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the particles increase epithelial cell regeneration in a colitis patient. In further embodiments, each of the one or more fusion proteins encapsulated by the particle comprises two or more antigenic epitopes selected from the group consisting of SEQ ID NOs: 4918-4941 and SEQ ID NOs: 4942-4952. In some embodiments, the particles increase re-myelination in a multiple sclerosis patient. In further embodiments, each of the one or more fusion proteins encapsulated by the particle comprises two or more antigenic epitopes derived from myelin basic protein and/or myelin oligodendrocyte glycoprotein. In further embodiments, each of said the or more fusion proteins encapsulated by the particle comprises two or more antigenic epitopes selected from the group consisting of: SEQ ID NOS: 2-1294.

Some aspects of the present invention provide methods for reducing the incidence and/or severity of an immune response to a therapeutic protein by a subject comprising administering to the subject an effective amount of a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the subject is undergoing enzyme replacement therapy for treatment of a disease selected from the group consisting of Hemophilia, Hemophilia A, Hemophilia B, von Willebrand disease, Gaucher's Disease, Fabry's Disease, Hurler's Disease, Pompe's Disease, Hunter's Disease, a mucopolysaccharide storage disorder, gangliosidosis, alkaline hypophosphatasia, cholesterol ester storage disease, hyperuricemia, growth hormone deficiency, renal anemia and Maroteaux-Lary Disease.

In further embodiments, the antigenic epitopes comprise one or more enzyme selected from the group consisting of Advate, antihemophilic factor, Kogenate, Eloctate, recombinant factor VIII Fc fusion protein, Refacto, Novo VIIa, recombinant factor VII, eptacog alfa, Helixate, Monanine, Coagulation Factor IX, Wilate, Ceredase, Alglucerase, Cerezyme, Imiglucerase, Elelso, taliglucerase alfa, Fabrazyme, Agalsidase beta, Aldurazyme, -I-iduronidase, Myozyme, Acid-glucosidase, Elaprase, iduronate-2-sulfatase, Naglazyme arylsufatase B, and N-acetylgalactosamine-4-sulfatase. In further embodiments, the antigenic epitopes comprise one or more protein selected from the group consisting of interferon-alpha, interferon-alpha 2a, interferon-beta Ib, interferon-beta Ia, insulin, DNAase, Neupogen, Epogen, Procrit (Epotein Alpha), Aranesp (2nd Generation Procrit), Intron A (interferon-alpha 2b), IL-2 (Proleukin), IL-I ra, BMP-7, TNF-alpha Ia, tPA, PDGF, interferon-gamma Ib, uPA, GMCSF, Factor VII, Factor VIII, Betaferon (interferon beta-Ia), somatotropin, and Rebif (interferon beta Ia).

In some embodiments, the therapeutic protein is an antibody, antigen-binding fragment, or Fc fragment thereof. In further embodiments, the antibody, antigen-binding fragment, or Fc fragment thereof is wherein the antibody or antigen-binding fragment thereof is Abciximab, Adalimumab, Adotrastuzumab emtansine, Alemtuzumab, Basiliximab, Bevacizumab, Belimumab, Blinatumomab, Brentuximab Vedotin, Canakinumab, Catumaxomab, Cetuximab, Certolizumab pegol, Daclizumab, Denosumab, Dinutuximab, Eculizumab, Efalizumab, Evolocumab, Gemtuzumab ozogamicin, Golimumab, Ibritumomab tiuxetan, Ipilimumab, Infliximab, Motavizumab, Muromonab, Natalizumab, Nivolumab, Obinutuzumab, Ofatumumab, Omalizumab, Panitumumab, Palivizumab, Pembrolizumab, Pertuzumab, Ramucirumab, Ranibizumab, Raxibacumab, Rituximab, Secukinumab, Siltuximab, Trastuzumab, Tocilizumab, Tositumomab-I-131, Ustekinumab, or Vedolizumab.

Some aspects of the present invention provide methods for increasing or inducing a protective immune response in a subject comprising administering an effective amount of a biodegradable particle described herein. In some embodiments, the method comprises administering to the subject an effective amount of a biodegradable particle comprising one or more fusion proteins encapsulated therein, wherein each one of the one or more fusion proteins comprises two or more antigenic epitopes, wherein the two or more antigenic epitopes are separated by a linker, wherein the linker comprises an amino acid sequence susceptible to specific cleavage, and wherein said biodegradable particle has a negative zeta potential. In some embodiments, the biodegradable particle is administered to the subject orally, intravenously, sublingually, buccally, enterically, topically, rectally, subcutaneously, nasally, intraosseously (i.e. intraosseous infusion), intraperitoneally, intrathecally, transdermally, or transmucosally. In some embodiments, the biodegradable particle is administered to the subject intravenously or subcutaneously. In further embodiments, the biodegradable particle is administered to the subject intravenously. In further embodiments, the biodegradable particle is administered to the subject subcutaneously.

Some aspects of the present invention provide methods for increasing or inducing a protective immune response in a subject comprising administering an effective amount of a biodegradable particle described herein, wherein the biodegradable particle is administered to the subject to treat or prevent a disease or condition. In some embodiments, the disease or condition is a cancer or an infectious disease. In some embodiments, the cancer is selected from the group consisting of a carcinoma, a lymphoma, a blastoma, a sarcoma such as liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma, a neuroendocrine tumor, mesothelioma, synovioma, schwannoma, meningioma, adenocarcinoma, melanoma, a leukemia, and a lymphoid malignancy. In some embodiments, the two or more antigenic epitopes each comprise at least a portion of a protein selected from the group consisting of CD19, CD20, BCMA, CD22, CLL1, CD33, CEA, CD123, CS1, EGFR, PSMA, EphA2, MCSP, ADAM17, PSCA, TPTE, HPU16, immature laminin receptor, TAG-72, HPV E6, HPV E7, BING-4, Calcium-activated chloride channel 2, cyclin B₁, 9D7, Ep-CAM, EphA3, Her2/neu, telomerase, mesothelin, SAP-1, survivin, proteins of the BAGE family, proteins of the CAGE family, proteins of the GAGE family, proteins of the MAGE family (e.g., MAGE-A3), proteins of the SAGE family, proteins of the XAGE family, CT9, CT10, NY-ESO1/LAGE-1, PRAME, SSX-2, Melan-A/MART-1, Cp100/pmel17, tyrosinase, TRP-1/TRP-2, P.polypeptide, MC1R, prostate-specific antigen, β-catenin, BRCA1/2, CDK4, CML66, fibronectin, MART-2, p53, Ras, TGF-βRII, and MUC1.

In some embodiments, the infectious disease is a bacterial, fungal, parasitic, or viral infection. In some embodiments, the viral infection is selected from the group consisting of a herpes virus infection, hepatitis virus infection, West Nile virus infection, flavivirus infection, influenza virus infection, rhinovirus infection, papillomavirus infection, paramyxovirus infection, parainfluenza virus infection, and/or a retrovirus infection. In some embodiments, the bacterial infection is selected from the group consisting of a Staphylococcus infection, Streptococcus infection, mycobacterial infection, Bacillus infection, Salmonella infection, Vibrio infection, Spirochete infection, and Neisseria infection.

Some aspects of the present invention provide a biodegradable particle comprising one or more fusion proteins encapsulated therein, wherein each one of said one or more fusion proteins comprises two or more antigenic epitopes selected from the group consisting of MOG₁₋₂₀, MBP₁₃₋₃₂, MOG₃₅₋₅₅, MBP₁₄₆₋₁₇₀, PLP₁₃₉₋₁₅₄, MBP₁₁₁₋₁₂₉, and MBP₈₃₋₉₉, wherein said two or more antigenic epitopes are separated by a linker, wherein said linker comprises an amino acid sequence susceptible to specific cleavage, wherein said biodegradable particle has a diameter of about 200 nm to 1000 nm, and wherein said biodegradable particle has a negative zeta potential of less than −30 mV.

Some aspects of the present invention provide methods of treating multiple sclerosis in a subject comprising administering to the subject an effective amount of a biodegradable particle comprising one or more fusion proteins encapsulated therein, wherein each one of said one or more fusion proteins comprises two or more antigenic epitopes selected from the group consisting of MOG₁₋₂₀, MBP₁₃₋₃₂, MOG₃₅₋₅₅, MBP₁₄₆₋₁₇₀, PLP₁₃₉₋₁₅₄, MBP₁₁₁₋₁₂₉, and MBP₈₃₋₉₉, wherein said two or more antigenic epitopes are separated by a linker, wherein said linker comprises an amino acid sequence susceptible to specific cleavage, wherein said biodegradable particle has a diameter of about 200 nm to 1000 nm, and wherein said biodegradable particle has a negative zeta potential of less than 30 mV. In some embodiments, the biodegradable particle is administered to the subject orally, intravenously, sublingually, buccally, enterically, topically, rectally, subcutaneously, nasally, intraosseously (i.e., intraosseous infusion), intraperitoneally, intrathecally, transdermally, or transmucosally. In some embodiments, the biodegradable particle is administered to the subject intravenously or subcutaneously. In further embodiments, the biodegradable particle is administered to the subject intravenously. In further embodiments, the biodegradable particle is administered to the subject subcutaneously.

### BRIEF DESCRIPTION OF THE DRAWINGS

FIG. 1 shows an exemplary fusion protein that is encapsulated into a biodegradable particle.

FIG. 2 illustrates the multi-peptide concept. A table of tolerogenic and control antigens, amino acid sequences, and molecular weights is shown (FIG. 2A). Also shown is a diagram highlighting the difference between one (top) and multiple (bottom) encapsulated peptides (FIG. 2B).

FIG. 3 shows physical analysis, including size distribution (FIG. 3A) and Zeta potential (FIG. 3B), of particles encapsulating PLP₁₃₉₋₁₅₁ as determined by light scattering.

FIG. 4 shows results of in vitro proliferation assays of PLG nanoparticles with DO11.10 transgenic T cells. FIG. 4A shows results of cells with nanoparticles alone. FIG. 4B shows results of cells with nanoparticles and 1 μg Ova323.

FIG. 5 shows results of in vitro proliferation assays of PLG nanoparticles with transgenic DO11.10 transgenic T cells. Shown are the results of nanoparticles alone (FIG. 5A), cell with nanoparticles only (FIG. 5B), cells with nanoparticles and 1 μg Ova₃₂₃ (FIG. 5C), and cells with nanoparticles and 1 μg/mL αCD28 (FIG. 5D).

FIG. 6 shows an inventory of particle batches encapsulating tolerogenic antigens individually (PLP₁₃₉₋₁₅₁, PLP₁₇₈₋₁₉₁, MBP₈₄₋₁₀₄, and MOG₉₂₋₁₀₆) or together (Tolerogenic together).

FIG. 7 shows an inventory of particle batches encapsulating control peptides individually (Ova₃₂₃₋₃₃₉, PLP₅₆₋₇₀, VP1₂₃₃₋₂₅₀, and VP2₇₀₋₈₆) or together (Control together).

FIG. 8 shows effects of PLG particles encapsulating control (OVA₃₂₃₋₃₃₉-PLG) or tolerogenic (PLP₁₃₉₋₁₅₁-PLG) peptides on splenic regulatory Type 1 T regulatory cell (TR1) populations. FIG. 8A shows percentages of LAG3⁺FoxP3⁻ cells in mice injected with PLG particles encapsulating control or tolerogenic peptides on Day 3 (left) and Day 5 (right), and the percentage of antigen-specific TR1 cells (LAG3⁺FoxP3⁻) that are also IFNγ⁺IL-10⁺. FIG. 8B shows the number of LAG3⁺FoxP3⁻ cells in mice treated with particles encapsulating control or tolerogenic peptides. FIG. 8C shows the number of LAG3⁺FoxP3⁻IFNγ⁺IL-10⁺ cells in mice treated with particles encapsulating control or tolerogenic peptides.

FIG. 9 shows data from a separate but duplicate experiment from FIG. 8. These data are different from the data shown in FIG. 8 in that the TR1s are not confirmed as FoxP3⁻ for this graph.

FIG. 10 shows flow cytometry results for splenic regulatory T cell populations following a transfer of naïve 5B6 (PLP₁₃₉₋₁₅₁ TCR transgenic mice) lymphocytes into näive SJL mice and treatment with either PLP₁₃₉₋₁₅₁-PLG particles or control OVA₃₂₃₋₃₃₉-SE PLG particles. All results are gated on CD90.1/Thy1.1 (PLP₁₃₉₋₁₅₁ TCR⁺) populations.

FIG. 11 shows T cell populations following transfer of CD4⁺ cells from 5B6 donors into näive SJL mice and treatment with either PLP₁₃₉₋₁₅₁-SE PLG particles or control OVA₃₂₃₋₃₃₉-SE PLG particles (d3 and d5). EAE was induced in a separate cohort (d5 post-EAE and d17 post-EAE). Shown are flow cytometry analyses for numbers antigen-specific T cells (FIG. 11A), proliferating antigen-specific T cells (FIG. 11B), antigen-specific regulatory T cells (FIG. 11C), and non-antigen specific regulatory T cells (FIG. 11D). All results are gated on CD90.1/Thy1.1 (PLP₁₃₉₋₁₅₁ TCR⁺) populations unless otherwise indicated (FIG. 11D).

FIG. 12 shows antigen-specific regulatory T cell expansion following transfer of CD4⁺ cells from DO11 (OVA₃₂₃₋₃₃₉ TCR transgenic mice) donors into naïve Balb/c RAG KO mice and treatment with either OVA₃₂₃₋₃₃₉-SE PLG particles or control PLP₁₃₉₋₁₅₁-SE PLG particles. Shown are flow cytometry analyses for the percentage (FIG. 12A) and numbers (FIG. 12B) of regulatory CD25⁺FoxP3⁺ T Cell populations. All results are gated on DO11 TCR⁺ populations.

FIG. 13 shows IFNγ-producing antigen-specific regulatory T cells following transfer of CD4⁺ cells from DO11 (OVA₃₂₃₋₃₃₉ TCR transgenic mice) donors into naïve Balb/c RAG KO mice and treatment with either OVA₃₂₃₋₃₃₉-SE PLG particles or control PLP₁₃₉₋₁₅₁-SE PLG particles. Shown are flow cytometry analyses of percentages (FIG. 13A) and numbers (FIG. 13B) of IFNγ-producing antigen-specific regulatory T cells.

FIG. 14 shows TR1 populations following transfer of CD4⁺ cells from DO11 (OVA₃₂₃₋₃₃₉ TCR transgenic mice) donors into naïve Balb/c RAG KO mice and treatment with either OVA₃₂₃₋₃₃₉-SE PLG particles or control PLP₁₃₉₋₁₅₁-SE PLG particles. Shown are flow cytometry analyses of percentages (FIG. 14A) and numbers (FIG. 14B).

FIG. 15 shows proliferation of antigen-specific regulatory T Cell populations after infusion of OVA₃₂₃₋₃₃₉-SE PLG particles or control PLP₁₃₉₋₁₅₁-SE PLG particles. Shown are flow cytometry results for Ki67⁺ DO11 TCR⁺ CD4⁺ cells (FIG. 15A), CD25⁺FoxP3⁺ gated on DO11 TCR⁺ CD4⁺ cells (FIG. 15B), and Ki67⁺CD49b⁺LAG3⁺ cells (FIG. 15C, from separate experiment, not confirmed as FoxP3⁻).

FIG. 16 shows the amino acid sequence of a PLP139-Ova323 fusion peptide linked by cathepsin-specific cleavage sites.

FIG. 17 shows results of an in vitro proliferation assay with the PLP139-Ova323 fusion peptide and DO11 (FIG. 17A) or 5B6 (FIG. 17B) cells.

FIG. 18 shows the characterization of three different attempts at encapsulation of the PLP139-Ova323 fusion peptide.

FIG. 19 shows the results of an in vitro proliferation assay DO11.10 transgenic T-cells treated with PLG(PLP139-Ova323) particles, PLG(Ova323) particles, or PLG(PLP139) particles. Shown are the results of cell treated with nanoparticles alone (FIG. 19A), nanoparticles and 1 μg Ova₃₂₃ (FIG. 19B), and nanoparticles, 1 μg Ova323, and 1 μg/mL αCD28 (FIG. 19C).

FIG. 20 shows the results of an in vitro proliferation assay with 5B6 transgenic T-cells treated with PLG(PLP139-Ova323) particles, PLG(Ova323) particles, or PLG(PLP139) particles. Shown are the results of cell treated with nanoparticles alone (FIG. 20A), nanoparticles and 1 μg PLP139 (FIG. 20B), and nanoparticles, 1 μg PLP139, and 1 μg/mL αCD28 (FIG. 20C).

FIG. 21 is a table showing the inventory of particle batches encapsulating PLP139-Ova323 fusion peptide.

FIG. 22 shows induction of EAE after administration of a PLP139-Ova323 fusion peptide, demonstrated by disease score (FIG. 22A) and mean ear swelling (FIG. 22B).

FIG. 23 shows amino acid sequences of four-epitope fusion peptides; EAE-1 linked epitopes, PLP139:PLP178:MOG92:MBP (FIG. 23A) and control linked epitopes, OVA323:PLP56:VP1-233:VP2-70 (FIG. 23B).

FIG. 24 shows characterization of nanoparticles encapsulating tolerogenic antigen fusion peptide (EAE-1 linked epitopes).

FIG. 25 is a table showing an inventory of particle batches encapsulating tolerogenic fusion peptide (EAE-1 linked epitopes) or negative control fusion peptide.

FIG. 26 shows the encapsulation efficiency of single EAE-specific epitopes and linked EAE-specific epitopes.

FIG. 27 shows the effects of encapsulated EAE-1 linked epitopes (EAE-1 tolerance treatment) on EAE disease score compared to encapsulated control linked epitopes (control linked epitope tolerance), OVA, and PLP139.

FIG. 28 demonstrates that FALK peptides induce EAE (FIG. 28A) and induce T cell proliferation (FIG. 28B).

FIG. 29 shows potential fusion peptides comprising neoepitopes, immune modulators, and TLR agonists.

FIG. 30 shows an outline of a protocol for development of linked-oncologic epitope proteins, subsequent nanoparticle encapsulation, and administration to patients.

FIG. 31 shows anticipated results of functional in vitro assays with peripheral blood monocytes treated with encapsulated NyEsol with or without anti-PD1. Shown are assays for the effects on T cell proliferation (FIG. 31A), IFNγ production (FIG. 31B), and IFNα production (FIG. 31C).

FIG. 32 shows anticipated survival curves for mice treated with encapsulated NyEsol with or without anti-PD1 in a mouse model of melanoma.

FIG. 33 shows exemplary in vitro functional assays for PBMCs treated with encapsulated, linked NY-ESO-1:Mage-A3:TPTE:Tyrosinase fusion proteins. Shown are anticipated results of T cell proliferation assays (FIG. 33A) and IFNγ production (FIG. 33B).

FIG. 34 shows potential fusion peptides comprising epitopes found in numerous disease and disorders.

### DETAILED DESCRIPTION OF THE INVENTION

The present inventors have found that nanoparticles encapsulating fusion proteins comprised of multiple peptide epitopes connected by cleavable linkers with specific protease sites can induce antigen-specific immune tolerance thus regulating the immune response in a multitude of disease models. In one embodiment, such particles are capable of decreasing the immune response to one or more of the peptide epitopes of the fusion protein, and are particularly useful in the treatment of diseases or conditions characterized by an excessive inflammatory immune response associated with more than one antigenic epitope, such as autoimmune diseases or allergies. In another embodiment, such particles are capable of inducing a protective immune response to one or more of the peptide epitopes of the fusion protein, and are particularly useful in the treatment of disease or conditions characterized by the absence of an immunogenic response, such as cancer.

As used in this specification and the appended claims, the singular forms “a,” “an” and “the” include plural references unless the content clearly dictates otherwise.

As used in this specification, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All publications and patents mentioned in the present application and/or listed below are herein incorporated by reference in their entireties.

### Fusion Proteins

Particular embodiments of the present invention are based, at least in part, on the novel discovery that particles encapsulating multiple antigens or epitopes can induce tolerance to each of these antigens when the antigens are linked together in a fusion protein by cleavable linkers. In some embodiments, the linkers are amino acid sequences that contain specific protease sites, and can be designed to allow for processing by the Class I pathway or the Class II pathway. In such embodiments, particle-encapsulated epitopes that are linked on the same fusion protein can be processed by both the class I and class II pathways. Thus, epitopes that are processed by the class I pathway can be linked in an encapsulated fusion protein with epitopes that are processed by the class II pathway

In some embodiments described herein, fusion proteins are encapsulated by biodegradable particles. The terms “fusion protein,” “fusion peptide,” “fusion polypeptide,” and “chimeric peptide” are used interchangeably herein and refer to one polypeptide chain created through the joining of two or more nucleotide sequences that originally encode for distinct proteins, or distinct parts of the same protein. Suitable fragments of an antigen for incorporation into the fusion protein described herein include any fragment of the full-length peptide that retains the function of generating the desired antigen-specific tolerance function of the present invention. “Fragment” refers to a portion of a protein. This portion contains, preferably, at least 10%, 20%, 30%, 40%, 50%, 60%, 70%, 80%, or 90% of the entire length of the reference sequence of the protein.

The fusion protein may be created by various means understood in the art (e.g., genetic fusion, chemical conjugation, etc.). The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. The two proteins may be fused either directly or via an amino acid linker. A peptide linker sequence may be employed to separate the first and second polypeptide components by a distance sufficient to ensure that each polypeptide folds into its secondary and tertiary structures. Amino acid sequences which may be usefully employed as linkers include those disclosed in Maratea et. al., Gene 40:39-46 (1985); Murphy et al., Proc. Natl. Acad. Sci. USA 83:8258-8262 (1986); U.S. Pat. Nos. 4,935,233 and 4,751,180; herein incorporated by reference in their entireties. The linker sequence may generally be from 1 to about 50 amino acids in length. In some embodiments, linker sequences are not required and/or utilized, for example, when the first and second polypeptides have non-essential N-terminal amino acid regions that can be used to separate the functional domains and prevent steric interference.

In a preferred embodiment, the individual antigens or epitopes are linked via an amino acid linker comprising a protease cleavage site that is specific for an intracellular protease (e.g., a protease present in the phagolysosome or cytosol of the cell). In some embodiments, the individual antigens or epitopes are linked by linkers comprising the same protease cleavage site. In some embodiments, the individual antigens or epitopes are linked by linkers comprising different protease cleavage sites. In further embodiments, one or more of the linker sequences in a fusion protein may comprise one or more protease cleavage sites.

Cleavage of the fusion protein by proteases located in the phagolysosome or cytosol directs the cleavage products (e.g., the individual peptide epitopes) for Class I or Class II antigen presentation. Class I antigen presentation is mediated by cytosolic proteases and major histocompatibility complex (MHC)-I and facilitates the presentation of intracellular proteins. As such, MHCI molecules typically present self-antigens or foreign proteins as a result of intracellular infection. Antigens presented in the context of MHCI are recognized by CD8⁺ T cells and typically lead to a cytotoxic response. Class II antigen presentation is mediated by phagocytosis of extracellular antigens, which are degraded by proteases present in the phagolysosome. Extracellular antigens are presented in the context of MHCII, and are recognized by CD4⁺ T cells. This recognition can lead to multiple downstream immune responses, such as Th1, Th2, Th17, Th22, or T regulatory responses depending on the nature of the antigen, the activation state of the antigen presenting cell, and the local cytokine microenvironment.

As such, introduction of specific cleavage sites allows for the regulation of the downstream immune response phenotype. For example, in the context of autoimmunity, it may be desirable to introduce cleavage sites for proteases present in the phagolysosome to increase the probability that the epitopes present in the fusion protein are presented in MHCII and lead to a regulatory or tolerogenic response. Alternatively, in the context of cancer therapeutics, it may be desirable to introduce cleavage sites for proteases present in the cytosol to increase the probability that the epitopes present in the fusion protein are presented in MHCI and result in a cytotoxic response that kills the cancerous cell.

The cleavage sites can be specific for any type of protease, such as serine proteases, cysteine proteases (e.g. cathepsins), metalloproteases, aspartic proteases, and others. In some embodiments, the cleavage sites are specific for cathepsin and/or furin proteases located in the phagolysosome. In some embodiments, the cleavage site is specific for any one or more of cathepsin proteases located in the phagolysosome such as cathepsin A, cathepsin B, cathepsin C, cathepsin D, cathepsin E, cathepsin F, cathepsin G, cathepsin H, cathepsin K, cathepsin L, cathepsin O, cathepsin W, or cathepsin Z. In a particular embodiment, the cleavage site is specific for cathepsin L. In some embodiments, the cleavage sites are specific for cathepsin and/or furin proteases located in the cytosol. In a particular embodiment, the cleavage site is specific for cathepsin S. In a further embodiment, the fusion protein comprises cleavage sites specific for cathepsin S and cathepsin L. In a particular embodiment, the linker sequence is Gly-Ala-Val-Val-Arg-Gly-Ala (SEQ ID NO: 5141).

As used herein, an “antigen” or “antigenic moiety” refers to any moiety, for example a peptide, that is recognized by the host's immune system. Examples of antigenic moieties include, but are not limited to, autoantigens, enzymes, and/or bacterial or viral proteins, peptides, drugs or components. An antigen may comprise one or more epitopes. As use herein, an “epitope” refers to a portion of an antigen recognized by an antibody or a T cell receptor. Not all epitopes are linear epitopes; epitopes can also be discontinuous, conformational epitopes. A number of discontinuous epitopes associated with autoimmune diseases or inflammatory diseases and/or disorders are known. In some embodiments, fusion proteins of the present invention comprise epitopes or antigens that have been previously described by PCT Application Publication No. WO 2015/023796, U.S. Patent Publication No. US 2015-0283218, and U.S. Patent Publication No. US 2015-0190485, each of which are hereby incorporated by reference in their entirety. Sequence identifiers used herein are consistent in their numbering with the sequence identifiers of U.S. Patent Publication No. US 2015-0190485.

In certain embodiments of this invention, the antigen or epitope is not in the same form as expressed in the subject being treated, but is a fragment or derivative thereof. Inducing antigens of this invention include peptides based on a molecule of the appropriate specificity but adapted by fragmentation, residue substitution, labeling, conjugation, and/or fusion with peptides having other functional properties. The adaptation may be performed for any desirable purposes, including but not limited to the elimination of any undesirable property, such as toxicity or immunogenicity; or to enhance any desirable property, such as mucosal binding, mucosal penetration, or stimulation of the tolerogenic arm of the immune response. Terms such as insulin peptide, collagen peptide, and myelin basic protein peptide, as used herein, refer not only to the intact subunit, but also to allotypic and synthetic variants, fragments, fusion peptides, conjugates, and other derivatives that contain a region that is homologous (preferably 70% identical, more preferably 80% identical and even more preferably 90% identical at the amino acid level) to at least 10 and preferably 20 consecutive amino acids of the respective molecule for which it is an analog, wherein the homologous region of the derivative shares with the respective parent molecule an ability to induce tolerance to the target antigen.

It is recognized that tolerogenic regions of an inducing antigen are often different from immunodominant epitopes, for example, for the stimulation of an antibody and/or T cell response. Tolerogenic regions are generally regions that can be presented in particular cellular interactions involving T cells. Tolerogenic regions may be present and capable of inducing tolerance upon presentation of the intact antigen. Some antigens contain cryptic tolerogenic regions, in that the processing and presentation of the native antigen does not normally trigger tolerance. An elaboration of cryptic antigens and their identification is found in International Patent Publication WO 94/27634.

In certain embodiments of this invention, fusion proteins are comprised of two, three, or a higher plurality of antigen or epitopes. It may be desirable to implement these embodiments when there is a plurality of target antigens.

Antigens can be prepared by a number of techniques known in the art, depending on the nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

In some embodiments, the antigens or epitopes are derived from a therapeutic antibody, antigen-binding fragment, or Fc fragment thereof. In some embodiments, the antigen is derived from a variant therapeutic antibody or antigen-binding fragment thereof that lacks functional complementarity determining regions (CDRs), In such embodiments, the antibody or antigen-binding fragment thereof may include a monoclonal antibody, a humanized monoclonal antibody, a human monoclonal antibody, a chimeric antibody, a single chain antibody, fragment antigen binding region (Fab), a single chain variable fragment (scFv), small modular immunopharmaceutical (SMIP), or a single antigen-binding domain. In some embodiments, the therapeutic antibody or antigen-binding fragment thereof binds to α4β1 integrin, Bacillus anthracis, B-L gamma S, C5, CD3, CD11a, CD20, CD25, CD30, CD33, CD52, CD59, CTLA4, EGFR, GD2, GPIIb, IIIa, HER2, IgE, IL-10, IL-5, IL12/23, PCSK9, PD1, RANK, RSV F protein, TNFα, or VEGF-A.

In some embodiments, the antigen is derived from a therapeutic antibody such as Abciximab, Adalimumab, Adotrastuzumab emtansine, Alemtuzumab, Basiliximab, Bevacizumab, Belimumab, Blinatumomab, Brentuximab Vedotin, Canakinumab, Catumaxomab, Cetuximab, Certolizumab pegol, Daclizumab, Denosumab, Dinutuximab, Eculizumab, Efalizumab, Evolocumab, Gemtuzumab ozogamicin, Golimumab, Ibritumomab tiuxetan, Ipilimumab, Infliximab, Motavizumab, Muromonab, Natalizumab, Nivolumab, Obinutuzumab, Ofatumumab, Omalizumab, Panitumumab, Palivizumab, Pembrolizumab, Pertuzumab, Ramucirumab, Ranibizumab, Raxibacumab, Rituximab, Secukinumab, Siltuximab, Trastuzumab, Tocilizumab, Tositumomab-I-131, Ustekinumab, or Vedolizumab.

In certain embodiments of this invention, the combination comprises a complex mixture of antigens obtained from a cell or tissue, one or more of which plays the role of inducing antigen. The antigens may be in the form of whole cells, either intact or treated with a fixative such as formaldehyde, glutaraldehyde, or alcohol. The antigens may be in the form of a cell lysate, created by detergent solubilization or mechanical rupture of cells or tissue, followed by clarification. The antigens may also be obtained by subcellular fractionation, particularly an enrichment of plasma membrane by techniques such as differential centrifugation, optionally followed by detergent solubilization and dialysis. Other separation techniques are also suitable, such as affinity or ion exchange chromatography of solubilized membrane proteins.

In one embodiment, the antigenic peptide or protein is an autoantigen, an alloantigen, neoantigen, oncoanitgen, or a transplantation antigen. In yet another particular embodiment, the autoantigen is selected from the group consisting of myelin basic protein, collagen or fragments thereof, DNA, nuclear and nucleolar proteins, mitochondrial proteins and pancreatic β-cell proteins. In some embodiments, one or more fusion proteins comprises the antigenic epitopes MOG₁₋₂₀, MBP₁₃₋₃₂, MOG₃₅₋₅₅, MBP₁₄₆₋₁₇₀, PLP₁₃₉₋₁₅s, MBP₁₁₁₋₁₂₉, and/or MBP₈₃₋₉₉. In some embodiments, the antigenic peptide or protein is gliadin or a gliadin epitope. In some embodiments, the antigen is one or more antigens selected from the group consisting of SEQ ID NOs: 1295-1724, SEQ ID NOs: 1726-1766 and SEQ ID NOs: 4986-5140.

The invention provides for the induction of tolerance to an autoantigen for the treatment of autoimmune diseases by administering the antigen for which tolerance is desired. For example, autoantibodies directed against the myelin basic protein (MBP) are observed in patients with multiple sclerosis, and, accordingly, MBP antigenic peptides or proteins may be used in the invention to be delivered using the compositions of the present invention to treat and prevent multiple sclerosis.

By way of another non-limiting example, a subject who is a candidate for a transplant from a non-identical twin may suffer from rejection of the engrafted cells, tissues or organs, as the engrafted antigens are foreign to the recipient. Prior tolerance of the recipient subject to the intended graft abrogates or reduces later rejection. Reduction or elimination of chronic anti-rejection therapies may be achieved by the practice of the present invention. In another example, many autoimmune diseases are characterized by a cellular immune response to an endogenous or self-antigen. Tolerance of the immune system to the endogenous antigen is desirable to control the disease.

In a further example, sensitization of a subject to an industrial pollutant or chemical, such as may be encountered on-the-job, presents a hazard of an immune response. Prior tolerance of the subject's immune system to the chemical/pollutant, in particular in the form of the chemical/pollutant reacted with the subject's endogenous proteins, may be desirable to prevent the later occupational development of an immune response.

Allergens are other antigens for which tolerance of the immune response thereto is also desirable. In one embodiment, the antigen is a gliadin or a gliadin epitope. In a further embodiment, the antigen is A-gliadin or an A-gliadin epitope. In some embodiments, the antigen is a mix of gliadins or gliadin epitopes. In further embodiments, the gliadins or a gliadin epitopes comprise one or more of SEQ ID NOs: 4983-4985.

Notably, even in diseases where the pathogenic autoantigen is unknown, bystander suppression may be induced using antigens present in the anatomical vicinity. For example, autoantibodies to collagen are observed in rheumatoid arthritis and, accordingly, a collagen-encoding gene may be utilized as the antigen-expressing gene module in order to treat rheumatoid arthritis (See e.g., Choy (2000) Curr Opin Investig Drugs 1: 58-62). Furthermore, tolerance to beta cell autoantigens may be utilized to prevent development of type 1 diabetes (See e.g., Bach and Chatenoud (2001) Ann Rev Immunol 19: 131-161).

As another example, auto-antibodies directed against myelin oligodendrocyte glycoprotein (MOG) are observed in autoimmune encephalomyelitis and in many other CNS diseases as well as multiple sclerosis (See e.g., Iglesias et al. (2001) Glia 36: 22-34). Accordingly, use of MOG antigen expressing constructs in the invention allows for treatment of multiple sclerosis as well as related autoimmune disorders of the central nervous system.

Still other examples of candidate autoantigens for use in treating autoimmune disease include: myelin basic protein, acetylcholine receptor, endogenous antigen, myelin oligodendrocyte glycoprotein, pancreatic beta-cell antigen, insulin, glutamic acid decarboxylase (GAD), collagen type 11, human cartilage gp39, fp130-RAPS, proteolipid protein, fibrillarin, small nucleolar protein, thyroid stimulating factor receptor, histones, glycoprotein gp70, pyruvate dehydrogenase dihydrolipoamide acetyltransferase (PCD-E2), hair follicle antigen, A-gliadin, gliadin, insulin, proinsulin, islet specific glucose-6-phosphatase catalytic subunit-related protein (IGRP), human tropomyosin isoform 5, Bahia grass pollen (BaGP), peach allergen Pru p 3, alpha s 1-Caein Milk allergen, Apigl celery allergen, Berel Brazil nut allergen, B-Lactoglobulin Milk allergen, Bovine serum albumin, Cor a 1.04 Hazelnut allergen, myelin associated glycoprotein, aquaporin α3 chain of type IV collagen, Ovalbumin Egg allergen, Advate, antihemophilic factor, Kogenate, Eloctate, recombinant factor VIII Fc fusion protein, Refacto, Novo VIIa, recombinant factor VII, eptacog alfa, Helixate, Monanine, Coagulation Factor IX, Wilate, Ceredase, Alglucerase, Cerezyme, Imiglucerase, Elelso, taliglucerase alfa, Fabrazyme, Agalsidase beta, Aldurazyme, -I-iduronidase, Myozyme, Acid-glucosidase, Elaprase, iduronate-2-sulfatase, Naglazyme arylsufatase B, or N-acetylgalactosamine-4-sulfatase pancreatic beta-cell antigens, insulin and GAD to treat insulin-dependent diabetes mellitus; collagen type 11, human cartilage gp 39 (HCgp39) and gpl30-RAPS for use in treating rheumatoid arthritis; myelin basic protein (MBP), proteolipid protein (PLP) and myelin oligodendrocyte glycoprotein (MOG, see above) to treat multiple sclerosis; fibrillarin, and small nucleolar protein (snoRNP) to treat scleroderma; thyroid stimulating factor receptor (TSH-R) for use in treating Graves' disease; nuclear antigens, histones, glycoprotein gp70 and ribosomal proteins for use in treating systemic lupus erythematosus; pyruvate dehydrogenase dihydrolipoamide acetyltransferase (PCD-E2) for use in treating primary biliary cirrhosis; hair follicle antigens for use in treating alopecia areata; and human tropomyosin isoform 5 (hTM5) for use in treating ulcerative colitis. In some embodiments, the antigens are selected from SEQ ID NOS: 2-1294.

Combinations can be humanized for their ability to promote tolerance by conducting experiments with isolated cells or in animal models.

In some embodiments, the tolerance inducing compositions of the present invention contain an apoptosis signaling molecule (e.g., in addition to a fusion protein). In some embodiments, the apoptosis signaling molecule is coupled and/or associated with the surface of the carrier. In some embodiments an apoptotic signaling molecules allows a carrier to be perceived as an apoptotic body by antigen presenting cells of the host, such as cells of the host reticuloendothelial system; this allows presentation of the associated peptide epitopes in a tolerance-inducing manner. Without being bound by theory, this is presumed to prevent the upregulation of molecules involved in immune cell stimulation, such as MHC class I/II, and costimulatory molecules. These apoptosis signaling molecules may also serve as phagocytic markers. For example, apoptosis signaling molecules suitable for the present invention have been described in U.S. Pat. No. 8,198,020, which is hereby incorporated by reference in its entirety. Molecules suitable for the present invention include molecules that target phagocytes, which include macrophages, dendritic cells, monocytes and neutrophils.

In some embodiments, molecules suitable as apoptotic signaling molecules act to enhance tolerance of the associated peptides. Additionally, a carrier bound to an apoptotic signaling molecule can be bound by Clq in apoptotic cell recognition (Paidassi et al., (2008) J. Immunol. 180:2329-2338; herein incorporated by reference in its entirety). For example, molecules that may be useful as apoptotic signaling molecules include rapamycin, phosphatidyl serine, annexin-1, annexin-5, milk fat globule-EGF-factor 8 (MFG-E8), or the family of thrombospondins (e.g., thrombospondin-1 (TSP-1)). Various molecules suitable for use as apoptotic signaling molecules with the present invention are discussed, for example, in U.S. Patent Publication No. 2012/0076831; herein incorporated by reference in its entirety.

In some embodiments, the fusion protein comprises one or more immune agonist. An immune agonist, as used herein, refers to a molecule that activates a particular immune signaling pathway, particularly an immunogenic signaling pathway. In some embodiments, the immune agonist activates a pattern recognition receptor such as a Toll-like receptor (TLR), C-type lectin receptor (CLR), NOD-like receptor, RIG-like receptor, or others. In particular embodiments, the agonist is a TLR agonist, such as a TLR1, TLR2, TLR3, TLR4, TLR5, TLR6, TLR7, TLR9, or TLR10 agonist. In such embodiments, the immune agonist facilitates the generation of an immunogenic response against one or more of the epitopes comprised within the fusion protein. Such embodiments are particularly useful in the context of vaccines and cancer immunotherapy.

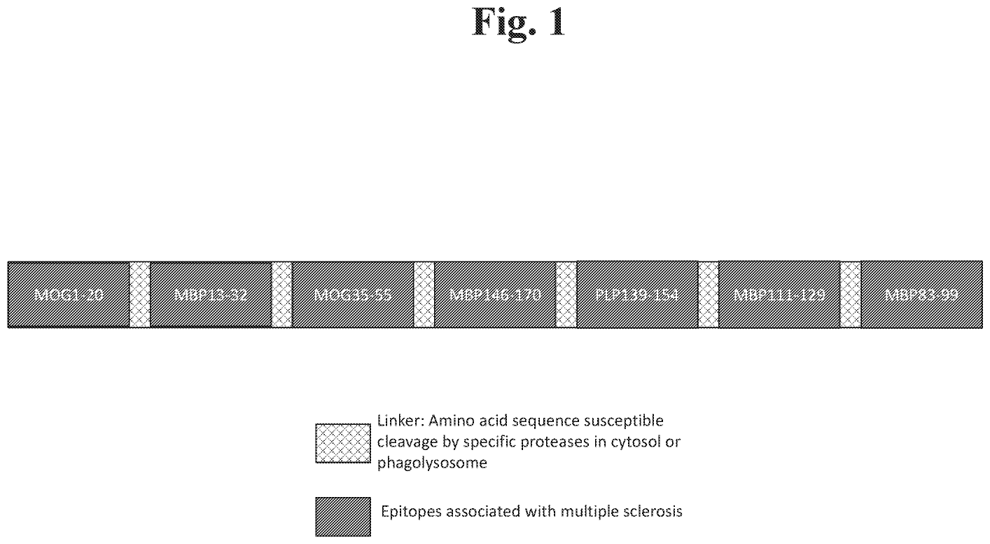

By way of example, and not intended to be limiting, a hypothetical exemplary fusion protein is shown in FIG. 1 and contains the epitopes MOG₁₋₂₀, MOG₃₅₋₅₅, MBP₁₃₋₃₂, MBP₈₃₋₉₉, MBP₁₁₁₋₁₂₉, MBP₁₄₆₋₁₇₀, and PLP₁₃₉₋₁₃₄, which are associated with multiple sclerosis (MS). The fusion protein is constructed by linking these 7 polypeptide epitopes together with specific linkers. These linkers are repeated amino acid sequences susceptible to cleavage by specific proteases. This protein will have a generic isoelectric point (PI) and solubility. When encapsulated into a particle, the particle will encapsulate the polypeptide epitopes at an equal ratio to each other.

### Biodegradable Particles

Certain embodiments are directed to biodegradable particles that encapsulate a fusion protein comprising two or more peptides, antigens, or epitopes connected by an amino acid linker sequence comprising specific protease sites. Particular embodiments contemplate that these particles are surprisingly effective at inducing tolerance to some or all of the linked peptides, antigens, or epitopes of the fusion proteins. Certain embodiments contemplate that the manufacture of these biodegradable particles is improved compared to biodegradable particles that encapsulate more than one peptide, antigen, and/or epitope that are not linked in a fusion protein

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term “particle” may be used to encompass beads and spheres. The particle may have any particle shape or conformation. However, in some embodiments it is preferred to use particles that are less likely to clump in vivo. Examples of particles within these embodiments are those that have a spherical shape.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups, including, but not limited to, poly(acrylic acid), Poly(ethylene-maleic anhydride) (PEMA), poly(vinyl alcohol) and sodium cholate.

In some embodiments, an antigenic peptide molecule is coupled to the carrier particle (e.g., immune modifying particle) by a conjugate molecule and/or linker group. In some embodiments, coupling of the antigenic peptide and/or apoptotic signaling molecule to the carrier particle (e.g., PLG particle) comprises one or more covalent and/or non-covalent interactions. In some embodiments, the antigenic peptide is attached to the surface of the carrier particle with a negative zeta potential. In some embodiments, the antigenic peptide is encapsulated within the carrier particle with a negative zeta potential. In some embodiments, the antigenic peptide is conjugated or linked to the carrier particle to produce an antigen-conjugated particle (See PCT Application No. PCT/US2016/068423, incorporated herein by reference in its entirety).

In one embodiment, the buffer solution contacting the immune modified particle may have a basic pH. Suitable basic pH for the basic solution include 7.1, 7.5, 8.0, 8.5, 9.5, 10.0, 10.5, 11.0, 11.5, 12.0, 12.5, 13.0, and 13.5. The buffer solution may also be made of any suitable base and its conjugate. In some embodiments of the invention, the buffer solution may include, without limitation, sodium bicarbonate, potassium bicarbonate, lithium bicarbonate, potassium dihydrogen phosphate, sodium dihydrogen phosphate, or lithium dihydrogen phosphate and conjugates thereof.

In one embodiment, the buffer solution contacting the immune modified particle may have an acidic pH. Suitable basic pH for the acidic solution include 4, 4.1, 4.2, 4.5, 5, 5.5, 6 and 6.5.

In one embodiment of the invention, the immune modified particles contain co-polymers. These co-polymers may have varying molar ratio. In some embodiments, the co-polymer ratio of the carrier particles described herein in 50:50. In further embodiments, suitable co-polymer ratio of the carrier particles described herein may be 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In another embodiment, the co-polymer may be periodical, statistical, linear, branched (including star, brush, or comb co-polymers) co-polymers. In some embodiments, the co-polymers ratio may be, but not limited to, polystyrene:poly(vinyl carboxylate)/80:20, polystyrene:poly(vinyl carboxylate)/90:10, poly(vinyl carboxylate):polystyrene/80:20, poly(vinyl carboxylate):polystyrene/90:10, polylactic acid:polyglycolic acid/80:20, or polylactic acid:polyglycolic acid/90:10.

In one embodiment, the particle is a liposome. In a further embodiment, the particle is a liposome composed of the following lipids at the following molar ratios—30:30:40 phosphatidylcholine:phosphatidylglycerol:cholesterol. In yet a further embodiment, the particle is encapsulated within a liposome.

It is not necessary that each particle be uniform in size, although the particles must generally be of a size sufficient to trigger phagocytosis in an antigen presenting cell or other MPS cell. Preferably, the particles are microscopic or nanoscopic in size, in order to enhance solubility, avoid possible complications caused by aggregation in vivo and to facilitate pinocytosis. Particle size can be a factor for uptake from the interstitial space into areas of lymphocyte maturation. A particle having a diameter of from about 0.1 μm to about 10 μm is capable of triggering phagocytosis. Thus in one embodiment, the particle has a diameter within these limits. In another embodiment, the particle has a diameter of about 0.3 μm to about 5 μm. In still another embodiment, the particle has a diameter of about 0.5 μm to about 3 μm. In a further embodiment, the particle has a diameter of about 0.2 μm to about 1 μm. In a further embodiment the particle has a diameter of about 0.1 μm, 0.2 μm, 0.3 μm, 0.4 μm, 0.5 μm, 1.0 μm, 1.5 μm, 2.0 μm, 2.5 μm, 3.0 μm, 3.5 μm, 4.0 μm, 4.5 μm, or about 5.0 μm. In a particular embodiment the particle has a size of about 0.5 μm. In some embodiments, the overall weights of the particles are less than about 10,000 kDa. In some embodiments, the weights of the particles are less than about 5,000 kDa, 1,000 kDa, 500 kDa, 400 kDa, 300 kDa, 200 kDa, 100 kDa, 50 kDa, 20 kDa, or less than about 10 kDa. The particles in a composition need not be of uniform diameter. By way of example, a pharmaceutical formulation may contain a plurality of particles, some of which are about 0.5 μm, while others are about 1.0 μm. Any mixture of particle sizes within these given ranges will be useful.

The particles of the current invention can possess a particular zeta potential. In certain embodiments, the zeta potential is negative. In one embodiment, the zeta potential is less than about −100 mV. In one embodiment, the zeta potential is less than about −50 mV. In certain embodiments, the particles possess a zeta potential between −100 mV and 0 mV. In a further embodiment, the particles possess a zeta potential between −75 mV and 0 mV. In a further embodiment, the particles possess a zeta potential between −60 mV and 0 mV. In a further embodiment, the particles possess a zeta potential between −50 mV and 0 mV. In still a further embodiment, the particles possess a zeta potential between −40 mV and 0 mV. In a further embodiment, the particles possess a zeta potential between −30 mV and 0 mV. In a further embodiment, the particles possess a zeta potential between −20 mV and 0 mV. In a further embodiment, the particles possess a zeta potential between −10 mV and 0 mV. In some embodiments, the particles possess a zeta potential between −80 mV and −30 mV. In a further embodiment, the particles possess a zeta potential between −80 mV and −20 mV. In a further embodiment, the particles possess a zeta potential between −80 mV and −10 mV. In a further embodiment, the particles possess a zeta potential between −70 mV and −30 mV. In a further embodiment, the particles possess a zeta potential between −70 mV and −20 mV. In a further embodiment, the particles possess a zeta potential between −70 mV and −10 mV. In a further embodiment, the particles possess a zeta potential between −60 mV and −30 mV. In a further embodiment, the particles possess a zeta potential between −60 mV and −20 mV. In a further embodiment, the particles possess a zeta potential between −60 mV and −10 mV. In a further embodiment, the particles possess a zeta potential between −50 mV and −30 mV. In a further embodiment, the particles possess a zeta potential between −50 mV and −20 mV. In a further embodiment, the particles possess a zeta potential between −50 mV and −10 mV. In a further embodiment, the particles possess a zeta potential between −50 mV and −40 mV. In a further embodiment, the zeta potential is less than about −30 mV.

In some embodiments, the charge of a carrier particle (e.g., positive, negative, neutral) is selected to impart application-specific benefits (e.g., physiological compatibility, beneficial surface-peptide interactions, etc.). In some embodiments, a carrier particle has a net neutral or negative charge (e.g., to reduce non-specific binding to cell surfaces which, in general, bear a net negative charge). In certain embodiments carrier particles are capable of being conjugated, either directly or indirectly, to an antigen to which tolerance is desired (also referred to herein as an antigen-specific peptide, antigenic peptide, autoantigen, inducing antigen or tolerizing antigen). In some instances, a carrier particle has multiple binding sites (e.g., 2, 3, 4, 5, 6, 7, 8, 9, 10, 20, 50, 100, or more) in order to have multiple copies of an antigen-specific peptide, or multiple different peptides, exposed on the surface (e.g., to increase the likelihood of a tolerance response). In some embodiments, a carrier particle displays a single type of antigenic peptide. In some embodiments, a carrier particle displays multiple different antigenic peptides on the surface. In some embodiments, a carrier particle surface displays functional groups for the covalent attachment of selected moieties (e.g., antigenic peptides). In some embodiments, carrier particle surface functional groups provide sites for non-covalent interaction with selected moieties (e.g., antigenic peptides). In some embodiments, a carrier particle has a surface to which conjugating moieties may be adsorbed without chemical bond formation.

In some embodiments, the particle is non-metallic. In these embodiments the particle may be formed from a polymer. In a preferred embodiment, the particle is biodegradable in a subject. In this embodiment, the particles can be provided in a subject across multiple doses without there being an accumulation of particles in the subject. Examples of suitable particles include polystyrene particles, PLGA particles, citric acid particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts point to the significance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles of the present invention may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22: 1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), etc.

Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

Suitable beads which are currently available commercially include polystyrene beads such as FluoSpheres (Molecular Probes, Eugene, Oreg.).

In some embodiments, the present invention provides systems comprising (a) a delivery scaffold configured for the delivery of chemical and/or biological agents to a subject; and (b) antigen-coupled poly(lactide-co-glycolide) particles for induction of antigen-specific tolerance. In some embodiments, at least a portion of said delivery scaffold is microporous. In some embodiments, the antigen-coupled poly(lactide-co-glycolide) particles are encapsulated within said scaffold. In some embodiments, the chemical and/or biological agents are selected from the group consisting of: protein, peptide, small molecules, nucleic acids, cells, and particles. In some embodiments, chemical and/or biological agents comprise cell, and said cells comprise pancreatic islet cells.

Physical properties are also related to a nanoparticle's usefulness after uptake and retention in areas having immature lymphocytes. These include mechanical properties such as rigidity or rubberiness. Some embodiments are based on a rubbery core, e.g., a poly(propylene sulfide) (PPS) core with an overlayer, e.g., a hydrophilic overlayer, as in PEG, as in the PPS-PEG system recently developed and characterized for systemic (but not targeted or immune) delivery. The rubbery core is in contrast to a substantially rigid core as in a polystyrene or metal nanoparticle system. The term rubbery refers to certain resilient materials besides natural or synthetic rubbers, with rubbery being a term familiar to those in the polymer arts. For example, cross-linked PPS can be used to form a hydrophobic rubbery core. PPS is a polymer that degrades under oxidative conditions to polysulphoxide and finally polysulphone, transitioning from a hydrophobic rubber to a hydrophilic, water-soluble polymer. Other sulphide polymers may be adapted for use, with the term sulphide polymer referring to a polymer with a sulphur in the backbone of the mer. Other rubbery polymers that may be used are polyesters with glass transition temperature under hydrated conditions that is less than about 37° C. A hydrophobic core can be advantageously used with a hydrophilic overlayer since the core and overlayer will tend not to mingle, so that the overlayer tends to sterically expand away from the core. A core refers to a particle that has a layer on it. A layer refers to a material covering at least a portion of the core. A layer may be adsorbed or covalently bound. A particle or core may be solid or hollow. Rubbery hydrophobic cores are advantageous over rigid hydrophobic cores, such as crystalline or glassy (as in the case of polystyrene) cores, in that higher loadings of hydrophobic drugs can be carried by the particles with the rubbery hydrophobic cores.

Another physical property is the surface's hydrophilicity. A hydrophilic material may have a solubility in water of at least 1 gram per liter when it is un-crosslinked. Steric stabilization of particles with hydrophilic polymers can improve uptake from the interstitium by reducing non-specific interactions; however, the particles' increased stealth nature can also reduce internalization by phagocytic cells in areas having immature lymphocytes. The challenge of balancing these competing features has been met, however, and this application documents the creation of nanoparticles for effective lymphatic delivery to DCs and other APCs in lymph nodes. Some embodiments include a hydrophilic component, e.g., a layer of hydrophilic material. Examples of suitable hydrophilic materials are one or more of polyalkylene oxides, polyethylene oxides, polysaccharides, polyacrylic acids, and polyethers. The molecular weight of polymers in a layer can be adjusted to provide a useful degree of steric hindrance in vivo, e.g., from about 1,000 to about 100,000 or even more; artisans will immediately appreciate that all the ranges and values within the explicitly stated ranges are contemplated, e.g., between 10,000 and 50,000.

The particles may incorporate functional groups for further reaction. Functional groups for further reaction include electrophiles or nucleophiles; these are convenient for reacting with other molecules. Examples of nucleophiles are primary amines, thiols, and hydroxyls. Examples of electrophiles are succinimidyl esters, aldehydes, isocyanates, and maleimides.

A great variety of means, well known in the art, may be used to conjugate antigenic peptides and proteins to carriers. These methods include any standard chemistries which do not destroy or severely limit the biological activity of the antigen peptides and proteins, and which allow for a sufficient number of antigen peptides and proteins to be conjugated to the carrier in an orientation which allows for interaction of the antigen peptide or protein with a cognate T cell receptor. Generally, methods are preferred which conjugate the C-terminal regions of an antigen peptide or protein, or the C-terminal regions of an antigen peptide or protein fusion protein, to the earner. The exact chemistries will, of course, depend upon the nature of the earner material, the presence or absence of C-terminal fusions to the antigen peptide or protein, and/or the presence or absence of conjugating moieties.

Functional groups can be located on the particle as needed for availability. One location can be as side groups or termini on the core polymer or polymers that are layers on a core or polymers otherwise tethered to the particle. For instance, examples are included herein that describe PEG stabilizing the nanoparticles that can be readily functionalized for specific cell targeting or protein and peptide drug delivery.

Conjugates such as ethylene carbodiimide (ECDI), hexamethylene diisocyanate, propyleneglycol di-glycidylether which contain 2 epoxy residues, and epichlorohydrin may be used for fixation of peptides or proteins to the carrier surface. Without being bound by theory, ECDI is suspected of carrying out two major functions for induction of tolerance: (a) it chemically couples the protein/peptides to the cell surface via catalysis of peptide bond formation between free amino and free carboxyl groups; and, (b) it induces the carrier to mimic apoptotic cell death such that they are picked up by host antigen presenting cells in the spleen and induce tolerance. It is this presentation to host T cells in a non-immunogenic fashion that leads to direct induction of anergy in autoreactive cells. In addition, ECDI serves as a potent stimulus for the induction of specific regulatory T cells.

In one series of embodiments, the antigen peptides and proteins are bound to the carrier via a covalent chemical bond. For example, a reactive group or moiety near the C-terminus of the antigen (e.g., the C-terminal carboxyl group, or a hydroxyl, thiol, or amine group from an amino acid side chain) may be conjugated directly to a reactive group or moiety on the surface of the carrier (e.g., a hydroxyl or carboxyl group of a PLA or PGA polymer, a terminal amine or carboxyl group of a dendrimer, or a hydroxyl, carboxyl or phosphate group of a phospholipid) by direct chemical reaction. Alternatively, there may be a conjugating moiety which covalently conjugates to both the antigen peptides and proteins and the carrier, thereby linking them together.

Reactive carboxyl groups on the surface of a carrier may be joined to free amines (e.g., from Lys residues) on the antigen peptide or protein, by reacting them with, for example, 1-ethyl-3-[3,9-dimethyl aminopropyl] carbodiimide hydrochloride (EDC) or N-hydroxysuccinimide ester (NHS). Similarly, the same chemistry may be used to conjugate free amines on the surface of a carrier with free carboxyls (e.g., from the C-terminus, or from Asp or Glu residues) on the antigen peptide or protein. Alternatively, free amine groups on the surface of a carrier may be covalently bound to antigen peptides and proteins, or antigen peptide or protein fusion proteins, using sulfo-SIAB chemistry, essentially as described by Arano et al. (1991) Chem. 2:71-6.

In another embodiment, a non-covalent bond between a ligand bound to the antigen peptide or protein and an anti-ligand attached to the carrier may conjugate the antigen to the carrier. For example, a biotin ligase recognition sequence tag may be joined to the C-terminus of an antigen peptide or protein, and this tag may be biotinylated by biotin ligase. The biotin may then serve as a ligand to non-covalently conjugate the antigen peptide or protein to avidin or streptavidin which is adsorbed or otherwise bound to the surface of the carrier as an anti-ligand. Alternatively, if the antigen peptides and proteins are fused to an immunoglobulin domain bearing an Fc region, as described above, the Fc domain may act as a ligand, and protein A, either covalently or non-covalently bound to the surface of the carrier, may serve as the anti-ligand to non-covalently conjugate the antigen peptide or protein to the carrier. Other means are well known in the art which may be employed to non-covalently conjugate antigen peptides and proteins to carriers, including metal ion chelation techniques (e.g., using a poly-His tag at the C-terminus of the antigen peptide or protein or antigen peptide or protein fusion proteins, and a Ni⁺-coated carrier), and these methods may be substituted for those described here.

Conjugation of a nucleic acid moiety to a platform molecule can be effected in any number of ways, typically involving one or more crosslinking agents and functional groups on the nucleic acid moiety and platform molecule. Linking groups are added to platforms using standard synthetic chemistry techniques. Linking groups can be added to nucleic acid moieties using standard synthetic techniques. The practitioner has a number of choices for antigens used in the combinations of this invention. The inducing antigen present in the combination contributes to the specificity of the tolerogenic response that is induced. It may or may not be the same as the target antigen, which is the antigen present or to be placed in the subject being treated which is a target for the unwanted immunological response, and for which tolerance is desired.

An inducing antigen of this invention may be a polypeptide, polynucleotide, carbohydrate, glycolipid, or other molecule isolated from a biological source, or it may be a chemically synthesized small molecule, polymer, or derivative of a biological material, providing it has the ability to induce tolerance according to this description when combined with the mucosal binding component.

In some embodiments, the present invention provides a carrier (e.g., immune modifying particle) coupled to one or more peptides, polypeptides, and/or proteins. In some embodiments, a carrier (e.g., PLG carrier), such as those described herein, are effective to induce antigen-specific tolerance and/or prevent the onset of an immune related disease (such as experimental autoimmune encephalomyelitis (EAE) in a mouse model) and/or diminish the severity of a pre-existing immune related disease. In some embodiments, the compositions and methods of the present invention can cause T cells to undertake early events associated with T-cell activation, but do not allow T-cells to acquire effector function. For example, administration of compositions of the present invention can result in T-cells having a quasi-activated phenotype, such as CD69 and/or CD44 upregulation, but do not display effector function, such as indicated by a lack of IFN-γ or IL-17 synthesis. In some embodiments, administration of compositions of the present invention results in T-cells having a quasi-activated phenotype without having conversion of naive antigen-specific T-cells to a regulatory phenotype, such as those having CD25⁺Foxp3⁺ phenotypes.

In some embodiments, the surface of a carrier (e.g., particle) comprises chemical moieties and/or functional groups that allow attachment (e.g., covalently, non-covalently) of antigenic peptides and/or other functional elements to the carrier. In some embodiments, the number, orientation, spacing, etc. of chemical moieties and/or functional groups on the carrier (e.g., particle) vary according to carrier chemistry, desired application, etc.

In some embodiments, a carrier comprises one or more biological or chemical agents adhered to, adsorbed on, encapsulated within, and/or contained throughout the carrier. In some embodiments, a chemical or biological agent is encapsulated in and/or contained throughout the particles. The present invention is not limited by the nature of the chemical or biological agents. Such agents include, but are not limited to, proteins, nucleic acid molecules, small molecule drugs, lipids, carbohydrates, cells, cell components, and the like. In some embodiments, two or more (e.g., 3, 4, 5, etc.) different chemical or biological agents are included on or within the carrier. In some embodiments, agents are configured for specific release rates. In some embodiments, multiple different agents are configured for different release rates. For example, a first agent may release over a period of hours while a second agent releases over a longer period of time (e.g., days, weeks, months, etc.). In some embodiments, the carrier or a portion thereof is configured for slow-release of biological or chemical agents. In some embodiments, the slow release provides release of biologically active amounts of the agent over a period of at least 30 days (e.g., 40 days, 50 days, 60 days, 70 days, 80 days, 90 days, 100 days, 180 days, etc.). In some embodiments, the carrier or a portion thereof is configured to be sufficiently porous to permit ingrowth of cells into the pores. The size of the pores may be selected for particular cell types of interest and/or for the amount of ingrowth desired.

Encapsulation of the antigen, biological, and/or chemical agents in the particle of the invention has been surprisingly found to induce immunological tolerance and has several advantages. First, the encapsulated particles have a slower cytokine response. Second, when using multiple antigens, biological, and/or chemical agents, encapsulation removes the competition between these various molecules that might occur if the agents were attached to the surface of the particle. Third, encapsulation allows more antigens, biological, and/or chemical agents to be incorporated with the particle. Fourth, encapsulation allows for easier use of complex protein antigens or organ homogenates (e.g., pancreas homogenate for type 1 diabetes or peanut extract in peanut allergy). Finally, encapsulation of antigens, biological, and/or chemical agents within the particle instead of conjugation to the surface of the particle maintains the net negative charge on the surface of the particle.

In some embodiments, the synthetic, biodegradable particles of the present invention provide ease of manufacturing, broad availability of therapeutic agents, and increased treatment sites. In particular embodiments, surface-functionalized biodegradable poly(lactide-co-glycolide) particles with a high density of surface carboxylate groups, synthesized using the surfactant poly(ethylene-alt-maleic anhydride) provide a carrier that offers numerous advantages over other carrier particles and/or surfaces. Experiments conducted during development of embodiments of the present invention demonstrated the conjugation of peptides (e.g., PLP₁₃₉₋₁₅₁ peptide) to these particles. Such peptide-coupled particles have shown that they are effective for the prevention of disease development and the induction of immunological tolerance (e.g., in the SJL/J PLP₁₃₉₋₁₅₁/CFA-induced R-EAE murine model of multiple sclerosis). Peptide coupled carriers of the present invention provide numerous advantages over other tolerance induction structures. In some embodiments, the particles are biodegradable, and therefore will not persist for long times in the body. The time for complete degradation can be controlled. In some embodiments, particles are functionalized to facilitate internalization without cell activation (e.g., phosphatidylserine loaded into PLG microspheres). In some embodiments, particles incorporate targeting ligands for a specific cell population. In some embodiments, anti-inflammatory cytokines such as IL-10 and TGF-β, are included on or within particles to limit activation of the cell type that is internalizing the particles and to facilitate the induction of tolerance via energy and/or deletion and the activation of regulatory T cells.

### Compositions

In some embodiments, the biodegradable particles encapsulating fusion proteins described herein may be formulated into a composition. As used herein, the term “composition” refers to a formulation of one or more particles encapsulating one or more fusion proteins that is capable of being administered to a subject and/or cell. In some embodiments, a composition may be comprised of a plurality of particles, each of which encapsulates the same fusion protein. In some embodiments, a composition may be comprised of a plurality of particles, each of which encapsulates one of two or more different fusion proteins. For example, a composition may be comprised of a plurality of particles each encapsulating one of 2, 3, 4, 5, 6, 7, 8, 9, 10 or more different fusion proteins. In some embodiments, compositions optionally further comprise one or more additional therapeutic agents. Alternatively, the particles of the current invention may be administered to a patient in need thereof in combination with the administration of one or more other therapeutic agents. For example, additional therapeutic agents for conjoint administration or inclusion in a pharmaceutical composition with a compound of this invention may be an approved anti-inflammatory agent, or it may be any one of a number of agents undergoing approval in the Food and Drug Administration that ultimately obtain approval for the treatment of any disorder characterized by an uncontrolled inflammatory immune response or a bacterial or viral infection. It will also be appreciated that particles of present invention can exist in free form for treatment, or where appropriate, as a pharmaceutically acceptable derivative thereof.

Composition formulations may include derivatives, prodrugs, solvates, stereoisomers, racemates, and/or tautomers of the particles described herein with any acceptable carriers, diluents, and/or excipients. A “therapeutic composition” or “pharmaceutical composition” (used interchangeably herein) is a composition of particles encapsulating one or more fusion proteins described herein capable of being administered to a patient and/or cell and resulting in a particular physiologic outcome (e.g., treatment of a particular disease or induction of antigen-specific tolerance).

As used herein “pharmaceutically acceptable carrier” or “pharmaceutically acceptable excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier suitable for use in contact with the tissues of human beings and animals without excessive toxicity, irritation, allergenicity, or other problems or complications. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the particles and/or fusion proteins of the present disclosure, its use in therapeutic compositions is contemplated.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, decanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like, and; metal chelating agents, such as citric acid, ethylenediamine tetraacetic acid (EDTA), sorbitol, tartaric acid, phosphoric acid, and the like.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, the present invention provides for methods of inducing a particular physiological effect (e.g., modulation of an immune response/induction of antigen-specific tolerance) in a subject comprising administering an effective amount of a biodegradable particle or composition described herein. Thus, in one aspect, tolerogenic immune modifying particles are provided. Such tolerance-inducing particles comprise, at a minimum, two or more antigenic epitopes against which induction of tolerance is desired (e.g., an autoantigen, an allergen, and/or a transplant antigen), separated by linkers (e.g., protease-specific linkers). In another aspect activating immune modifying particles are provided. In one embodiment, such immune activating particles comprise two or more antigenic epitopes against which a protective immune response is desired (e.g., tumor antigens and/or infectious agents), separated by linkers (e.g., protease-specific linkers). In a particular embodiment, the activating immune modifying particles further comprise an immune activating agent. In one embodiment, the immune activating agent is a TLR agonist. In a particular embodiment, the immune activating agent is a TLR7, TLR3, or a TLR9 agonist. In a further embodiment, the immune activating agent is a TLR7 agonist.

Compositions may be formulated in a particular manner suitable for a desired administration route and/or to achieve a desired outcome. “Administration” refers to introducing or delivering a biodegradable particle or composition thereof to a subject or contacting a biodegradable particle or composition thereof with a cell or a sample. The term “sample” refers to a volume and/or mass obtained, provided, and/or subjected to analysis. In some embodiments, a sample comprises a tissue sample, cell sample, a fluid sample, and the like. In some embodiments, a sample is taken from a subject (e.g., a human or animal subject). In some embodiments, a tissue sample comprises a portion of tissue taken from any internal organ, a cancerous, pre-cancerous, or non-cancerous tumor, skin, hair (including roots), eye, muscle, bone marrow, cartilage, white adipose tissue, or brown adipose tissue. In some embodiments, a fluid sample comprises buccal swabs, blood, cord blood, saliva, semen, urine, ascites fluid, pleural fluid, spinal fluid, pulmonary lavage, tears, sweat, and the like. Those of ordinary skill in the art will appreciate that, in some embodiments, a “sample” is a “primary sample” in that it is obtained directly from a subject. In some embodiments, a “sample” is the result of processing of a primary sample, for example to remove certain potentially contaminating components and/or to isolate and/or purify certain components of interest.

Administration can occur by injection, irrigation, inhalation, consumption, electro-osmosis, hemodialysis, iontophoresis, and other methods known in the art. The particles and compositions of the present invention may be administered via any acceptable route including, but not limited to, orally, intravenously, sublingually, buccally, enterically, topically, rectally, subcutaneously, nasally, intraosseously (e.g., by intraosseous infusion), intraperitoneally, intrathecally, transdermally, or transmucosally. In particular embodiments, the particles of the invention are administered intravenously or subcutaneously.

The frequency of administration may be determined based on the desired physiologic outcome, the nature of the disorder to be treated and/or prevented, the severity of the disorder, and the subject's response to the formulation. In some aspects, administration of a composition occurs at least once. In further aspects, administration occurs more than once, for example 2, 3, 4, 5, 6, 7, 8, 9, 10 or more times in a given period. The dosage of each administration and/or frequency of administrations may be adjusted as necessary based on the patient's condition and physiologically responses. Where compositions are administered more than once, each administration may be performed by the same actor and/or in the same geographical location. Alternatively, each administration may be performed by a different actor and/or in a different geographical location.

In some embodiments, an effective amount of a particle and/or composition described herein is administered to a subject. The terms “subject” and “patient” are used interchangeably herein and refer to animals (e.g., mammals, swine, fish, birds, insects etc.) suitable for treatment with the particles and/or compositions described herein. In some embodiments, subjects are mammals, such as primates, humans, or rabbits; livestock, such as cattle, sheep, goats, cows, swine, and the like; poultry, such as chickens, ducks, geese, turkeys, and the like; domesticated animals, such as dogs and cats; rodents, such as mice, rats, or hamsters. In some embodiments, particularly in research contexts, the subject is a mouse. In some embodiments, the subject is a human.

The term “effective amount” refers the minimum amount of a biodegradable particle or composition required to induce a particular physiological effect. For example, an effective amount may be the minimum amount required to induce antigen-specific tolerance or to otherwise regulate an immune response. As described herein, regulation of an immune response may be humoral and/or cellular, and is measured using standard techniques in the art and as described herein. The effective amount of a given particle or composition depends on a variety of factors including the nature of the disorder being treated and the severity of the disorder; activity of the specific particle(s) or composition(s) employed; the age, body weight, general health, sex and diet of the subject; the time of administration, route of administration, and rate of excretion of the particle(s) or composition(s) employed; the duration of the treatment; drugs used in combination or coincidental with the particle(s) or composition(s) employed; the judgment of the prescribing physician or veterinarian; the size and physical characteristic of the particles or compositions; and like factors known in the art. Useful dosage ranges of the particles or compositions described herein may be, for example, from about any of the following: 0.5 to 10 mg/kg, 1 to 9 mg/kg, 2 to 8 mg/kg, 3 to 7 mg/kg, 4 to 6 mg/kg, 5 mg/kg, 1 to 10 mg/kg, 5 to 10 mg/kg. Alternatively, the dosage can be administered based on the number of particles. For example, useful dosages of the carrier, given in amounts of carrier delivered, may be, for example, about 10⁶, 10⁷, 10⁶, 10⁹, 10¹⁰, or greater number of particles per dose. The absolute amount given to each patient depends on pharmacological properties such as bioavailability, clearance rate and route of administration. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa, USA., which is hereby incorporated by reference in its entirety.

In some embodiments, compositions of the present invention find use with one or more scaffolds, matrices, and/or delivery systems (See e.g., U.S. Pat. App. 2009/0238879; U.S. Pat. Nos. 7,846,466; 7,427,602; 7,029,697; 6,890,556; 6,797,738; 6,281,256; herein incorporated by reference in their entireties). In some embodiments, particles are associated with, adsorbed on, embedded within, conjugated to, etc. a scaffold, matrix, and/or delivery system (e.g., for delivery of chemical/biological material, cells, tissue, and/or an organ to a subject). In some embodiments, a scaffold, matrix, and/or delivery system (e.g., for delivery of chemical/biological material, cells, tissue, and/or an organ to a subject) comprises and/or is made from materials described herein.

In some embodiments, microporous scaffolds (e.g., for transplanting biological material (e.g., cells, tissue, etc.) into a subject) are provided. In some embodiments, microporous scaffolds are provided having thereon agents (e.g., extracellular matrix proteins, exendin-4) and biological material (e.g., pancreatic islet cells). In some embodiments, the scaffolds are used in the treatment of diseases (e.g., type 1 diabetes), and related methods (e.g., diagnostic methods, research methods, drug screening). In some embodiments, scaffolds are provided with the carrier particles described herein on and/or within the scaffold. In some embodiments, scaffolds are produced from antigen conjugated materials (e.g., antigen conjugated PLG).

In some embodiments, a scaffold and/or delivery system comprises one or more layers and/or has one or more chemical and/or biological entities/agents (e.g., proteins, peptide-conjugated particles, small molecules, cells, tissue, etc.), see, e.g., U.S. Patent Publication No. 2009/0238879; herein incorporated by reference in its entirety. In some embodiments, the particles described herein are co-administered with a scaffold delivery system to elicit induction of immunological tolerance to the scaffold and the associated materials. In some embodiments, microporous scaffold is administered to a subject with particles described herein on or within the scaffold. In some embodiments, particles described herein are coupled to a scaffold delivery system. In some embodiments, a scaffold delivery system comprises any of the carrier particles described herein.

It will also be appreciated that the particles and compositions of the present invention can be formulated and employed in combination therapies, that is, the particles and compositions can be formulated with or administered concurrently with, prior to, or subsequent to, one or more other desired therapeutics or medical procedures. The particular combination of therapies (e.g., combinations of therapeutic compound, and/or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects).

In certain embodiments, the pharmaceutical compositions containing the modified particles of the present invention further comprise one or more additional therapeutically active ingredients (e.g., anti-inflammatory and/or palliative). For purposes of the invention, the term “palliative” refers to treatment that is focused on the relief of symptoms of a disease and/or side effects of a therapeutic regimen, but is not curative. For example, palliative treatment encompasses painkillers, anti-nausea medications and anti-sickness drugs.

In some embodiments, compositions described herein are administered along with (e.g., concurrent with, prior to, or following) an implant (e.g., device) and/or transplant (e.g., tissue, cells, organ) in order to mediate, negate, regulate and/or reduce the immune response associated with the implant and/or transplant.

### Methods of Use

In some embodiments, the present invention provides methods for inducing or otherwise regulating an existing immune response in a subject, preferably a mammal, more preferably a human, comprising administering to the subject the particles or compositions described herein. As used herein, the term “immune response” includes both innate immune responses and adaptive immune responses (e.g., T cell mediated and/or B cell mediated immune responses). In general, innate and adaptive immune responses are distinguished by the level of antigen specificity. For example, cells directly involved in the adaptive immune response (e.g., T cells and B cells) express T cell receptors (TCRs) and B cell receptors that are specific for a particular antigen. Thus, adaptive immune receptors are activated and respond to specific antigens (e.g., a specific epitope or component of a larger antigen). Conversely, cells of the innate immune express innate immune receptors (e.g. pattern recognition receptors (PRRs)) such as TLRs, CLRs, NLRs, RLRs, and others. PRRs are germ-line encoded and non-rearranging receptors that recognize broader types of antigens (e.g., CLRs generally recognize carbohydrate moieties, RLRs recognize viral nucleic acids). Thus, receptors of the innate immune system are activated and respond to a broad range of antigens and are not considered to be antigen-specific.

Cells involved in the immune response include lymphocytes, such as B cells and T cells (CD4⁺, CD8⁺, Th1, Th2, Th17, T regulatory cells); antigen presenting cells (APCs) including professional APCs such as dendritic cells, macrophages, B lymphocytes, Langerhans cells, and nonprofessional APCs such as keratinocytes, endothelial cells, astrocytes, fibroblasts, oligodendrocytes; natural killer cells, and; myeloid cells, such as macrophages, eosinophils, mast cells, basophils, and other granulocytes. Exemplary immune responses include T cell responses, e.g., T cell proliferation, T cell expansion, cytokine production, chemokine production, and T cell-mediated cellular cytotoxicity (e.g., responses mediated by CD8⁺ cytotoxic T cells (CTLs)). In addition, the term immune response includes immune responses that indirectly or directly mediate T cell activation or T cell suppression, such as migration, proliferation, and activation of APCs, and mechanisms of antigen presentation. The term immune response also includes immune responses that are indirectly affected by T cell activation, e.g., antibody production (humoral responses) and activation of cytokine responsive cells, e.g., macrophages, dendritic cells, neutrophils, mast cells, basophils, B cells, T cells themselves, and structural cells such as epithelial cells, endothelial cells, and/or other stromal cells. In some embodiments, the particles of the present invention are effective to reduce inflammatory cell trafficking to the site of inflammation.

“Regulating an immune response” may refer to the modulation of any aspect, or modulation of multiple aspects, of an immune response. In some embodiments, methods for regulating an immune response as provided herein include modulating an immunogenic, pro-inflammatory, or otherwise activating immune response (e.g., through the use of activating immune modifying particles). In such embodiments, the methods provided herein encompass specifically inducing a TH1, TH2, or TH17 response, reducing or inhibiting a T regulatory response, or a combination of these responses. Induction of a TH1 response encompasses increasing the expression of, e.g., IFNγ and/or IL-12, and/or increasing a population of TH1 cells (e.g., increasing the number or percentage of IFNγ⁺, IL-12⁺, and/or T-bet⁺ cells). Induction of a TH2 response encompasses increasing the expression of, e.g., IL-4, IL-5, IL-10, IL-13, or any combination thereof. Typically an increase in a TH2 response will comprise an increase in the expression of at least one of IL-4, IL-5, IL-10, or IL-13; more typically an increase in a TH2 response will comprise an increase in the expression of at least two of IL-4, IL-5, IL-10, or IL-13, most typically an increase in a TH2 response will comprise an increase in the expression of at least three of IL-4, IL-5, IL-10, or IL-13, while ideally an increase in a TH2 response will comprise an increase in the expression of all of IL-4, IL-5, IL-10, and IL-13. Induction of a TH2 response may also comprise increasing a population of TH2 cells (e.g., increasing the number or percentage of IL-4⁺, IL-5⁺, IL-10⁺, IL-13⁺, and/or GATA3⁺ cells). Induction of a TH17 response encompasses increasing the expression of, e.g., TGF-β, IL-6, IL-21, IL-23 or any combination thereof, and effects levels of IL-17, IL-21 and IL-22. Induction of a TH17 response may also comprise increasing a population of TH17 cells (e.g., increasing the number or percentage of IL-17⁺, IL-21⁺, IL-22⁺, and/or RORγt⁺ cells). Reduction of a T regulatory response encompasses decreasing expression of TGFβ, IL-10, or any combination thereof. Reduction of a T regulatory response may also comprise a reduction in a population of T regulatory cells (e.g., decreasing the number or percentage of TGFβ⁺, IL-10⁺, and/or FoxP3⁺ cells).

In some embodiments, methods for regulating an immune response as provided herein include modulating a regulatory, tolerogenic, or otherwise suppressive immune response (e.g., through the use of tolerogenic immune modifying particles). In such embodiments, the methods provided herein encompass specifically reducing a TH1, TH2, or TH17 response, increasing a T regulatory response, or a combination of these responses. Reduction of a TH1 response encompasses decreasing the expression of, e.g., IFNγ and/or IL-12, and/or decreasing a population of TH1 cells (e.g., decreasing the number or percentage of IFNγ⁺, IL-12⁺, and/or T-bet⁺ cells). Reduction of a TH2 response encompasses decreasing the expression of, e.g., IL-4, IL-5, IL-10, IL-13, or any combination thereof. Typically, decreasing a TH2 response will comprise a decrease in the expression of at least one of IL-4, IL-5, IL-10, or IL-13; more typically a decrease in a TH2 response will comprise a decrease in the expression of at least two of IL-4, IL-5, IL-10, or IL-13, most typically a decrease in a TH2 response will comprise a decrease in the expression of at least three of IL-4, IL-5, IL-10, or IL-13, while ideally a decrease in a TH2 response will comprise a decrease in the expression of all of IL-4, IL-5, IL-10, and IL-13. Reduction of a TH2 response may also comprise decreasing a population of TH2 cells (e.g., decreasing the number or percentage of IL-4⁺, IL-5⁺, IL-10⁺, IL-13⁺, and/or GATA3⁺ cells). Reduction of a TH17 response encompasses decreasing the expression of, e.g., TGF-β, IL-6, IL-21, IL-23 or any combination thereof, and effects levels of IL-17, IL-21 and IL-22. Reduction of a TH17 response may also comprise decreasing a population of TH17 cells (e.g., decreasing the number or percentage of IL-17⁺, IL-21⁺, IL-22⁺, and/or RORγt⁺ cells). Induction of a T regulatory response encompasses increasing the expression of TGFβ and/or IL-10. Induction of a T regulatory response may also comprise increasing a population of T regulatory cells (e.g., increasing the number or percentage of TGFβ⁺, IL-10⁺, and/or FoxP3⁺ cells).

As used herein, the terms “tolerance” or “immunological tolerance” refer to a state of unresponsiveness of the immune system. Immunological tolerance is critical in preventing aberrant (e.g., reactivity to autoantigens in the context of autoimmunity) and/or excessive immune responses. “Specific” immunological tolerance occurs when immunological tolerance is preferentially invoked against certain antigens in comparison with others. “Non-specific” immunological tolerance occurs when immunological tolerance is invoked indiscriminately against antigens which lead to an inflammatory immune response. “Quasi-specific” immunological tolerance occurs when immunological tolerance is invoked semi-discriminately against antigens which lead to a pathogenic immune response but not to others which lead to a protective immune response. In particular embodiments, the present invention provides for methods of inducing antigen-specific tolerance in a subject comprising administering an effective amount of a biodegradable particle or composition described herein. As used herein, “antigen-specific tolerance” refers to the insensitivity and/or unresponsiveness of a T cell to TCR-mediated stimulation by particular antigens.

Immunological tolerance is a result of both central and peripheral tolerance. Central tolerance refers to the positive and negative selection of T cells in the thymus that results in the selection of functional, antigen-specific T cells (positive selection) and the elimination of autoreactive T cells (negative selection). Peripheral tolerance refers to tolerance mechanisms present in the periphery (e.g., bone marrow, lymph nodes, spleen, and/or mucosal surfaces). Mechanisms of peripheral tolerance prevent aberrant responses by autoreactive T cells that have escaped thymic deletion and prevent excessive activation of immune responses to foreign antigens. Peripheral tolerance encompasses a variety of mechanisms including T cell anergy, activation-induced cell death of T cells, and mechanisms of immune suppression mediated by regulatory T cells.

As used herein, the term “anergy” refers to insensitivity of T cells to T cell receptor (TCR)-mediated stimulation. Such insensitivity is antigen-specific and generally persists after exposure to the antigenic peptide has ceased. T-cell anergy occurs when T cells are exposed to antigen and receive a first signal (e.g., a TCR- or CD3-mediated signal) in the absence of a second signal (e.g., a costimulatory signal). Under these conditions, re-exposure of the cells to the same antigen, even if re-exposure occurs in the presence of a costimulatory molecule, results in failure of the T cell to produce cytokines (e.g., IL-2) and subsequent failure to proliferate. Thus, a failure to produce cytokines prevents proliferation. Anergic T cells can, however, proliferate if cultured with cytokines (e.g., IL-2). T cell anergy can be observed by the lack of IL-2 production by T cells as measured by ELISA, or by a proliferation assay using an indicator cell line. Alternatively, a reporter gene construct can be used. For example, anergic T cells fail to initiate DL-2 gene transcription induced by a heterologous promoter under the control of the 5′ IL-2 gene enhancer or by a multimer of the API sequence that can be found within the enhancer (Kang et al. 1992 Science. 257:1134).

The terms “regulatory T cells,” “T regulatory cells,” and “T regs” are used interchangeably herein and T cells that suppress or prevent the induction of an immune response. The term T reg can refer to both natural (e.g., T regs that emerge from the thymus as suppressive cells) and induced Tregs (e.g., T regs that differentiate into suppressive cells in response to peripheral stimuli). Induced T regs can be divided into multiple sub-populations based on their expression of the transcription factor, FoxP3, cell surface markers, and cytokine production. In some embodiments, T regs may refer to induced T reg populations such as Tr1, Th3, CD8⁺ suppressor cells, and others. In particular embodiments, T regs may refer to Tr1 cells, which are defined as CD4⁺FoxP3⁻LAG3⁺IFNγ⁺IL-10⁺. In some embodiments, T reg-mediated suppression of an immune response may be antigen-specific or non-antigen specific. In some embodiments, T reg mediated suppression of an immune response may be a result of cytokine production by the T reg (e.g., the production of IL-10 and/or TGFβ) or by the production of another immunosuppressive mediator.

T regs play an important role in mediating and maintaining peripheral tolerance. See, for example, Walker et al. (2002) Nat. Rev. Immunol. 2: 11-19; Shevach et al. (2001) Immunol. Rev. 182:58-67. In some situations, peripheral tolerance to an autoantigen is lost (or broken) and an autoimmune response ensues. For example, in an animal model for EAE, activation of APCs through innate immune receptors such as Toll-like receptors was shown to break self-tolerance and result in the induction of EAE (Waldner et al. (2004) J. Clin. Invest. 113:990-997). Additionally, T regs can prevent excessive immune activation in the context of beneficial immune responses, such as those generated in response to a viral or bacterial infection. The regulation of these immune responses can therefore prevent excessive damage to healthy cells or tissues.

In some embodiments, immunological tolerance can be measured by decreased level of a specific immune response, for example those mediated at least in part by antigen-specific effector T lymphocytes, B lymphocytes, antibodies, or their equivalents; a delay in the onset or progression of a specific immune response; or a reduced risk of the onset or progression of a specific immune response. Immunological tolerance can be determined by methods performed on a proportion of treated subjects in comparison with untreated subjects, wherein T cell and/or B cell proliferation and/or activation, cytokine production, antibody production can be determined by methods known in the art (e.g., in vitro proliferation assays, flow cytometry, ELISA, Western blots, etc.).

In some embodiments, induction of an antigen-specific immune response comprises inducing an increase in tolerogenic activity. In some embodiments, an increase in tolerogenic activity includes expansion and/or proliferation of T regs. In some embodiments, an increase in tolerogenic activity includes increased production of regulatory cytokines such as IL-10 and/or TGFβ. A proxy for tolerogenic activity is the ability of an intact antigen or fragment to stimulate the production of an appropriate cytokine at the target site. The immunoregulatory cytokine released by T regulatory cells at the target site is thought to be TGF-β (Miller et al., Proc. Natl. Acad. Sci. USA 89:421, 1992). Other factors that may be produced during tolerance are the cytokines IL-4 and IL-10, and the mediator PGE. In contrast, lymphocytes in tissues undergoing activating immune responses secrete cytokines such as IL-1, IL-2, IL-6, and IFNγ. Hence, the ability of an antigen to induce a tolerogenic or an immunogenic response can be evaluated by measuring its ability to stimulate the production of immunoregulatory cytokines (e.g., TGFβ and/or IL-10) in comparison to immune-activating cytokines (e.g., IFNγ, IL-2, IL-6, IL-17, etc.).

In certain embodiments, the present invention relates to the priming of immune tolerance in a subject not previously tolerized by therapeutic intervention. In some embodiments, the invention relates to methods for reducing the incidence and/or severity of an aberrant immune response to a therapeutic protein in a subject. These embodiments generally involve a plurality of administrations of a combination of antigen and mucosal binding component. Typically, at least three administrations, frequently at least four administrations, and sometimes at least six administrations are performed during priming in order to achieve a long-lasting result, although the subject may show manifestations of tolerance early in the course of treatment. Most often, each dose is given as a bolus administration, but sustained formulations capable of mucosal release are also suitable. Where multiple administrations are performed, the time between administrations is generally between 1 day and 3 weeks, and typically between about 3 days and 2 weeks. Generally, the same antigen and mucosal binding component are present at the same concentration, and the administration is given to the same mucosal surface, but variations of any of these variables during a course of treatment may be accommodated.

In some embodiments, the methods of the present invention comprise inducing a protective immune response to a particular antigen, such as a target antigen. Such methods are particularly useful in the context of cancer therapeutics and infectious disease. In such embodiments, the methods comprise administration of particles encapsulating a fusion protein that includes two or more target antigens (such as a tumor antigen) separated by protease-specific linkers. In certain embodiments, the particles encapsulating linked epitopes further comprise an immune agonist. An immune agonist may comprise any of a protein, a hapten, a toxin, a lipid, and/or a nucleic acid and is capable of acting as an adjuvant to result in antigen-specific immune responses against the target antigen. An immune agonist may comprise a hapten, such as biotin, dinitrophenol, urushiol, fluoroscein and others. In some embodiments, an immune agonist may comprise a nucleic acid, including single-stranded (ss) and double stranded RNA and DNA, and modified forms thereof. In some embodiments, an immune agonist is a toxin. In some embodiments, the immune agonist is a protein such as an immune activating cytokine (e.g. IL-2, IL-12, IFNγ, IFNα, IFNβ, TNFα, etc.); a chemokine capable of recruiting T cells, antigen presenting cells, and/or granulocytes; an antibody or fragment thereof that binds to and inhibits an immune checkpoint receptor (e.g., PD1, PDL1, CTLA4, LAG3, TIM3, or A2aR).

In some embodiments, the immune agonist is an agonist of a CLR (e.g., DEC-205, DC-SIGN, DCIR, CLEC-1, Dectin1, Dectin2, or DLEC), a TLR (e.g., TLR1, TLR2, TLR3, TLR4, TLR5, TLR6, TLR7, TLR8, TLR9, TLR10, TLR11, TLR12, or TLR13), an NLR (e.g. NOD1, NOD2, NAIP, NLRC4, NLRC3, NLPR1, NLPR3, NLRP10), an RLR (e.g., MDA or, RIG1), STING, an inflammasome (e.g., NLPR3 or AIM2). In further embodiments, the immune agonist is a TLR7 or a TLR9 agonist. In further particular embodiments, the immune agonist results in the activation of CD8⁺ CTLs. In further embodiments, the immune agonist results in the cell-lysis of cells expressing the target antigen. In some embodiments, the target antigen is a tumor antigen such as CD19, CD20, BCMA, CD22, CLL1, CD33, CEA, CD123, CS1, EGFR, PSMA, EphA2, MCSP, ADAM17, PSCA, TPTE, HPU16, immature laminin receptor, TAG-72, HPV E6, HPV E7, BING-4, Calcium-activated chloride channel 2, cyclin B₁, 9D7, Ep-CAM, EphA3, Her2/Neu, telomerase, mesothelin, SAP-1, survivin, proteins of the BAGE family, proteins of the CAGE family, proteins of the GAGE family, proteins of the MAGE family (e.g., MAGE-A3), proteins of the SAGE family, proteins of the XAGE family, CT9, CT10, NY-ESO1/LAGE-1, PRAME, SSX-2, Melan-A/MART-1, Cp100/pmel17, tyrosinase, TRP-1/TRP-2, P.polypeptide, MC1R, prostate-specific antigen, β-catenin, BRCA1/2, CDK4, CML66, fibronectin, MART-2, p53, Ras, TGF-βRII, and MUC1.

Increased antigen-specific immune responses may be measured by increased antigen-specific effector T cell proliferation, increased production of pro-inflammatory and/or immune activating cytokines (e.g., IFNγ or IFNα), or increased cell lysis of cells expressing the target antigen.

In further embodiments, methods are provided for the treatment of a particular disease or disorder. “Treating” and “treatment” as used herein refer to an improvement of a disease, or a symptom of a disease, and may be a measureable or observable improvement, or an improvement in the general well-being of the subject. In particular embodiments, treating a particular disease or disorder refers to inducing antigen-specific tolerance or otherwise increasing a regulatory immune response to decrease or ameliorate pathologic inflammation (e.g., in the context of an autoimmune disease).

In some embodiments, the invention relates to uses of the particles and compositions described herein prior to the onset of disease. In other embodiments, the invention relates to uses of the particles and compositions described herein to inhibit ongoing disease. In some embodiments, the invention relates to ameliorating disease in a subject. By ameliorating disease in a subject is meant to include treating, preventing or suppressing the disease in the subject.

In some embodiments, the invention relates to preventing the relapse of disease. For example, an unwanted immune response can occur at one region of a peptide (such as an antigenic determinant). Relapse of a disease associated with an unwanted immune response can occur by having an immune response attack at a different region of the peptide. T-cell responses in some immune response disorders, including MS and other Th1/17-mediated autoimmune diseases, can be dynamic and evolve during the course of relapsing-remitting and/or chronic-progressive disease. The dynamic nature of the T cell repertoire has implications for treatment of certain diseases, since the target may change as the disease progresses. Previously, pre-existing knowledge of the pattern of responses was necessary to predict the progression of disease. The present invention provides compositions that can prevent the effect of dynamic changing disease, a function of “epitope spreading.” A known model for relapse is an immune reaction to proteolipid protein (PLP) as a model for multiple sclerosis (MS). Initial immune response can occur by a response to PLP139-15. Subsequent disease onset can occur by a relapse immune response to PLP[pi]s-iβi. The compositions of the present invention are particularly useful for the treatment of MS and other autoimmune diseases where disease-causing epitopes are present in multiple proteins (e.g., PLP, MBP, and MOG) or where multiple disease-causing epitopes reside on a single protein and encapsulation of the whole protein is not otherwise possible.

In certain embodiments, the subject suffers from a disorder associated with unwanted immune activation, such as allergic disease or condition, allergy, and asthma. A subject having an allergic disease or asthma is a subject with a recognizable symptom of an existing allergic disease or asthma. Tolerance can be induced in such a subject, for example, by particles complexed with the specific foods (e.g., peanut proteins, etc.), injected substances (e.g., bee venom proteins, etc.), or inhaled substances (e.g., ragweed pollen proteins, pet dander proteins, etc.) which elicit the allergic reaction.

In certain embodiments, the subject suffers from a disorder associated with unwanted immune activation, such as autoimmune disease and inflammatory disease. A subject having an autoimmune disease or inflammatory disease is a subject with a recognizable symptom of an existing autoimmune disease or inflammatory disease. Tolerance can be induced in such a subject, for example, by particles complexed with the relevant autoantigens driving the particular autoimmune disease.

In certain embodiments, the subject suffers from a disorder associated with enzyme replacement therapy. Tolerance can be induced in such a subject, for example, by particles complexed with the enzymes which patients with genetic deficiencies fail to produce, to prevent them from making neutralizing antibody responses to recombinantly-produced enzymes administered to treat their particular deficiency, e.g. tolerance to human Factor VIII in patients with hemophilia due to a genetic deficiency in the ability to make Factor VIII.

In certain embodiments, the subject suffers from a disorder associated with disease therapy. In the case of recombinant antibodies, tolerance is induced for example, to a humanized antibody being employed in a therapeutic context to prevent a patient from making neutralizing antibodies against the antibody therapeutic, e.g. tolerance to a humanized immune subset depleting antibody or anti-cytokine antibody being used as a treatment for autoimmune disease.

Autoimmune diseases can be divided in two broad categories: organ-specific and systemic. Autoimmune diseases include, without limitation, rheumatoid arthritis (RA), systemic lupus erythematosus (SLE), type I diabetes mellitus, type II diabetes mellitus, multiple sclerosis (MS), immune-mediated infertility such as premature ovarian failure, scleroderma, Sjogren's disease, vitiligo, alopecia (baldness), polyglandular failure, Grave's disease, hypothyroidism, polymyositis, pemphigus vulgaris, pemphigus foliaceus, inflammatory bowel disease including Crohn's disease and ulcerative colitis, autoimmune hepatitis including that associated with hepatitis B virus (HBV) and hepatitis C virus (HCV), hypopituitarism, graft-versus-host disease (GvHD), myocarditis, Addison's disease, autoimmune skin diseases, uveitis, pernicious anemia, Celiac disease, and hypoparathyroidism.

Autoimmune diseases may also include, without limitation, Hashimoto's thyroiditis, Type I and Type II autoimmune polyglandular syndromes, paraneoplastic pemphigus, bullous pemphigoid, dermatitis herpetiformis, linear IgA disease, epidermolysis bullosa acquisita, erythema nodosa, pemphigoid gestationis, cicatricial pemphigoid, mixed essential cryoglobulinemia, chronic bullous disease of childhood, hemolytic anemia, thrombocytopenic purpura, Goodpasture's syndrome, autoimmune neutropenia, myasthenia gravis, Eaton-Lambert myasthenic syndrome, stiff-man syndrome, acute disseminated encephalomyelitis, Guillain-Barre syndrome, chronic inflammatory demyelinating polyradiculoneuropathy, multifocal motor neuropathy with conduction block, chronic neuropathy with monoclonal gammopathy, opsoclonus-myoclonus syndrome, cerebellar degeneration, encephalomyelitis, retinopathy, primary biliary sclerosis, sclerosing cholangitis, gluten-sensitive enteropathy, ankylosing spondylitis, reactive arthritides, polymyositis/dermatomyositis, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiitis obliterans, sarcoidosis, glomerulonephritis, and cryopathies. These conditions are well known in the medical arts and are described, for example, in Harrison's Principles of Internal Medicine, 14th ed., Fauci A S et al., eds., New York: McGraw-Hill, 1998.

Animal models for the study of autoimmune disease are known in the art. For example, animal models which appear most similar to human autoimmune disease include animal strains which spontaneously develop a high incidence of the particular disease. Examples of such models include, but are not limited to, the non-obese diabetic (NOD) mouse, which develops a disease similar to type 1 diabetes, and lupus-like disease prone animals, such as New Zealand hybrid, MRL-Fas^{lpr} and BXSB mice. Animal models in which an autoimmune disease has been induced include, but are not limited to, EAE (a murine model of multiple sclerosis), collagen-induced arthritis (CIA, a murine model of rheumatoid arthritis), and experimental autoimmune uveitis (EAU, a murine model of uveitis). Animal models for autoimmune disease have also been created by genetic manipulation and include, for example, IL-2/IL-10 knockout mice for inflammatory bowel disease, Fas or Fas ligand knockout for SLE, and IL-1 receptor antagonist knockout for rheumatoid arthritis.

In certain embodiments, the subject suffers from an infection. A subject having a bacterial, fungal, parasitic, or viral infection is a subject with a recognizable symptom of an existing bacterial, fungal, parasitic, or viral infection. Infectious agents include, but are not limited to, bacterial, fungal, parasitic, and viral agents. Examples of such infectious agents include the following: staphylococcus, methicillin-resistant Staphylococcus aureus, Escherichia coli, streptococcaceae, neisseriaaceae, cocci, enterobacteriaceae, enterococcus, vancomycin-resistant enterococcus, cryptococcus, histoplasmosis, aspergillus, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, candida, systemic mycoses, opportunistic mycoses, protozoa, nematodes, trematodes, cestodes, adenoviruses, herpesviruses (including, for example, herpes simplex virus and Epstein Barr virus, and herpes zoster virus), poxviruses, papovaviruses, hepatitis viruses, (including, for example, hepatitis B virus and hepatitis C virus), papilloma viruses, orthomyxoviruses (including, for example, influenza A, influenza B, and influenza C), paramyxoviruses, coronaviruses, picomaviruses, reoviruses, togaviruses, flaviviruses, bunyaviridae, rhabdoviruses, rotavirus, respiratory syncitial virus, human immunodeficiency virus and retroviruses. Exemplary infectious diseases include but are not limited to candidiasis, candidemia, aspergillosis, streptococcal pneumonia, streptococcal skin and oropharyngeal conditions, gram negative sepsis, tuberculosis, mononucleosis, influenza, respiratory illness caused by Respiratory Syncytial Virus, malaria, schistosomiasis, and trypanosomiasis.

In some embodiments, the viral infection is a herpes virus infection, hepatitis virus infection, West Nile virus infection, flavivirus infection, influenza virus infection, rhinovirus infection, papillomavirus infection, paramyxovirus infection, parainfluenza virus infection, and/or a retrovirus infection. Preferred viruses are those viruses that infect the central nervous system of the subject. Most preferred viruses are those that cause encephalitis or meningitis.

In some embodiments, the bacterial infection is a Staphylococcus infection, Streptococcus infection, mycobacterial infection, Bacillus infection, Salmonella infection, Vibrio infection, Spirochete infection, and Neisseria infection. Preferred are bacteria that infect the central nervous system of the subject. Most preferred are bacteria that cause encephalitis or meningitis.

Other embodiments of this invention relate to transplantation. Transplantation refers to the transfer of a sample or graft from a donor subject to a recipient subject, and is frequently performed on human recipients who need the tissue in order to restore a physiological function provided by the tissue. Tissues that are transplanted include (but are not limited to) whole organs such as kidney, liver, heart, lung; organ components such as skin grafts and the cornea of the eye; and cell suspensions such as bone marrow cells and cultures of cells selected and expanded from bone marrow or circulating blood, and whole blood transfusions.

A serious potential complication of any transplantation ensues from antigenic differences between the host recipient and the engrafted tissue. Depending on the nature and degree of the difference, there may be a risk of an immunological assault of the graft by the host, or of the host by the graft, or both, may occur. The extent of the risk is determined by following the response pattern in a population of similarly treated subjects with a similar phenotype, and correlating the various possible contributing factors according to well accepted clinical procedures. The immunological assault may be the result of a preexisting immunological response (such as preformed antibody), or one that is initiated about the time of transplantation (such as the generation of TH cells). Antibody, TH cells, or Tc cells may be involved in any combination with each other and with various effector molecules and cells. However, the antigens which are involved in the immune response are generally not known, therefore posing difficulties in designing antigen-specific therapies or inducing antigen-specific tolerance.

Certain embodiments of the invention relate to decreasing the risk of host versus graft disease, leading to rejection of the tissue graft by the recipient. The treatment may be performed to prevent or reduce the effect of a hyperacute, acute, or chronic rejection response. Treatment is preferentially initiated sufficiently far in advance of the transplant so that tolerance will be in place when the graft is installed; but where this is not possible, treatment can be initiated simultaneously with or following the transplant. Regardless of the time of initiation, treatment will generally continue at regular intervals for at least the first month following transplant. Follow-up doses may not be required if a sufficient accommodation of the graft occurs, but can be resumed if there is any evidence of rejection or inflammation of the graft. Of course, the tolerization procedures of this invention may be combined with other forms of immunosuppression to achieve an even lower level of risk.

In some embodiments, the present invention provides methods of treating cancer in a subject. “Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer.

In some embodiments, the present invention provides methods of treating allergy in a subject. “Allergy,” as used herein, includes all immune reactions mediated by IgE as well as those reactions that mimic IgE-mediated reactions. Allergies are induced by allergens, including proteins, peptides, carbohydrates, and combinations thereof, that trigger an IgE or IgE-like immune response. Allergies include food allergies (e.g., nut, milk, egg, fish, shellfish, wheat, or soy allergies). Exemplary food allergens include Ara h 1, Ara h 2, and Ara h 3 epitopes in peanuts; the 15 kd antigen in celery; apple antigen Mal d 1; Pru p3 in peach, and; α-gliadin and γ-gliadin epitopes in gluten. Allergies also include other environmental allergies (e.g., pollen, insect sting, dust, mold, fungal allergies, etc.). Exemplary environmental allergens include urushiol in poison ivy and oak; house dust antigen; birch pollen components Bet v 1 and Bet v 2; Timothy grass pollen allergen Phl p 1; Lol p 3, Lol p I, or Lol p V in Rye grass; Cyn d 1 in Bermuda grass; dust mite allergens dust mite Der p1, Der p2, or Der f1; bee venom phospholipase A2, and; Japanese cedar pollen

Various modification, recombination, and variation of the described features and embodiments will be apparent to those skilled in the art without departing from the scope and spirit of the invention. Although specific embodiments have been described, it should be understood that the invention as claimed should not be unduly limited to such specific embodiments. Indeed, various modifications of the described modes and embodiments that are obvious to those skilled in the relevant fields are intended to be within the scope of the following claims.

### EXAMPLES

The following examples are provided to further illustrate the advantages and features of the invention, but are not intended to limit the scope of this disclosure.

### Example 1—Characterization of PLG Particles Encapsulating Peptide Epitopes

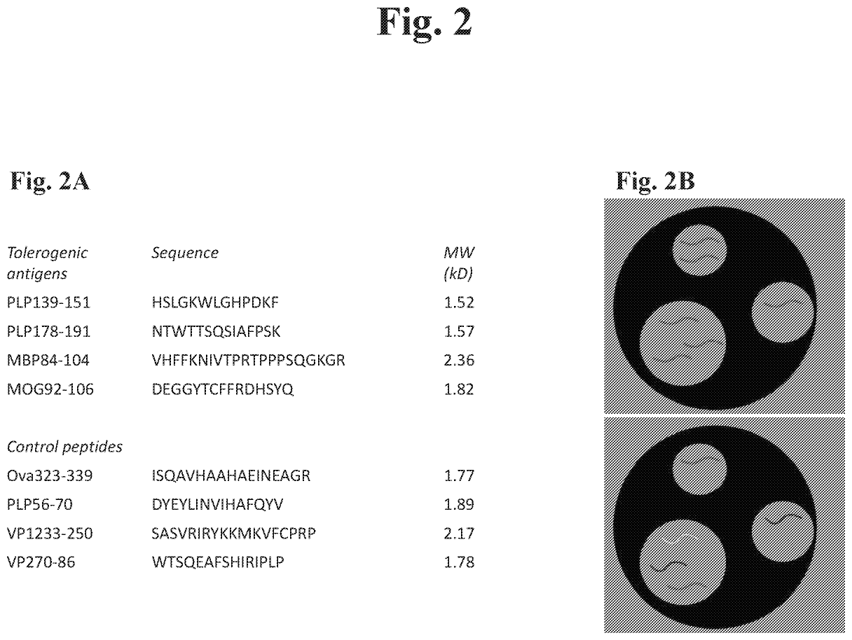

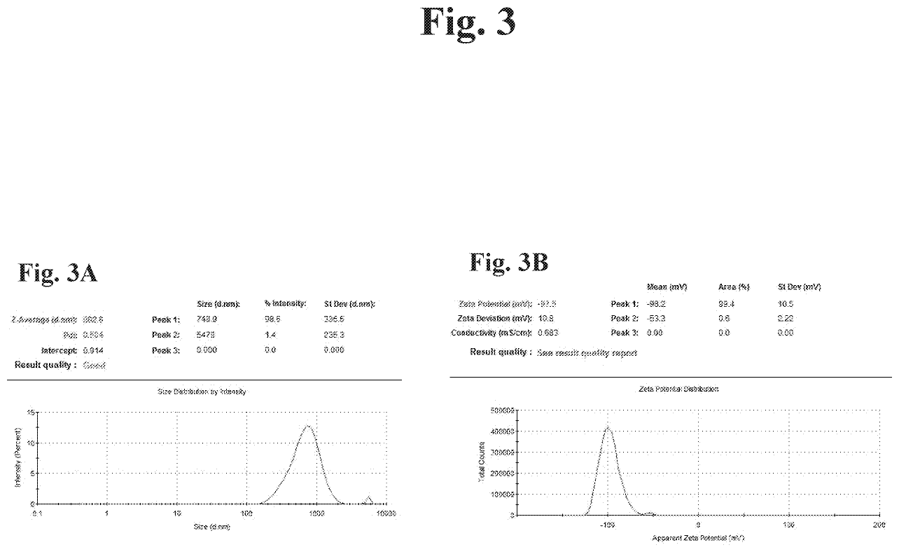

Experiments were performed to determine the physical and functional characteristics of PLG particles encapsulating different peptide epitopes. A listing of tolerogenic and control epitopes is shown in FIG. 2. As an example, PLG particles encapsulating PLP₁₃₉₋₁₅₁ were generated and the size distribution and Zeta potential were determined by light scattering. The peak of the size distribution for PLG particles encapsulating PLP₁₃₉₋₁₅₁ was 748.9 nm and the Z-average size was 882.6 nm, indicating the presence of larger species as well (FIG. 3A). Further, the PLG particles encapsulating PLP₁₃₉₋₁₅₁ had a very negative charge (Z-potential=−97.5 mV, FIG. 3B), likely due to the use of poly(ethylene-co-maleic acid) (PEMA) as an emulsifying agent.

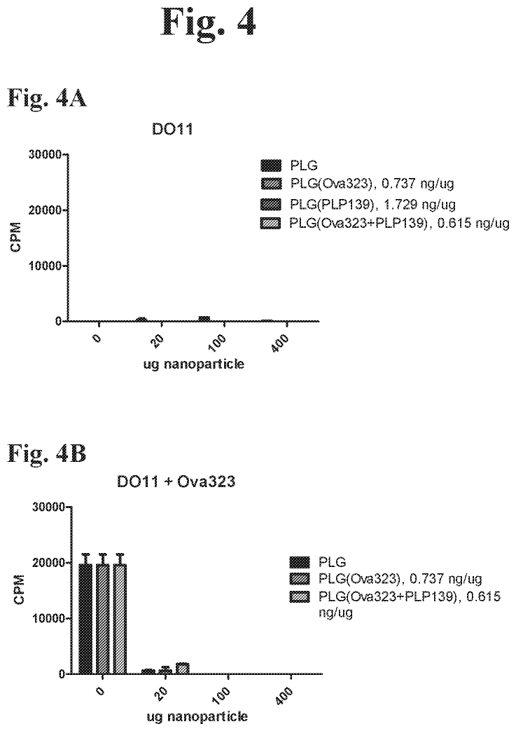

The effects of PLG nanoparticles encapsulating different peptide epitopes on cell proliferation were then determined. 2×10⁵ Splenocytes from DO11.10 transgenic mice (TCR transgenic mice specific for Ova323) were incubated with PLG nanoparticles encapsulating PLP₁₃₉₋₁₅₁ (0.737 ng/μg) or Ova₃₂₃₋₃₃₉ (1.729 ng/μg) individually or PLG nanoparticles encapsulating PLP₁₃₉₋₁₅₁ and Ova₃₂₃₋₃₃₉ together (0.615 ng/μg). Nanoparticles and splenocytes were plated with or without Ova323 (1 μg) and allowed to grow for 2 days, then pulsed with thymidine and cultured for an additional 3 days. Exposure of cells to Ova323 in the absence of nanoparticles resulted in increased cell proliferation (FIG. 4B). However, treatment of cells exposed to Ova323 with nanoparticles encapsulating Ova₃₂₃₋₃₃₉ individually or nanoparticles encapsulating PLP₁₃₉₋₁₅₁ and Ova₃₂₃₋₃₃₉ together significantly reduced cell proliferation (FIG. 4B). Cells that were not exposed to Ova323 did not proliferate (FIG. 4A, negative control).

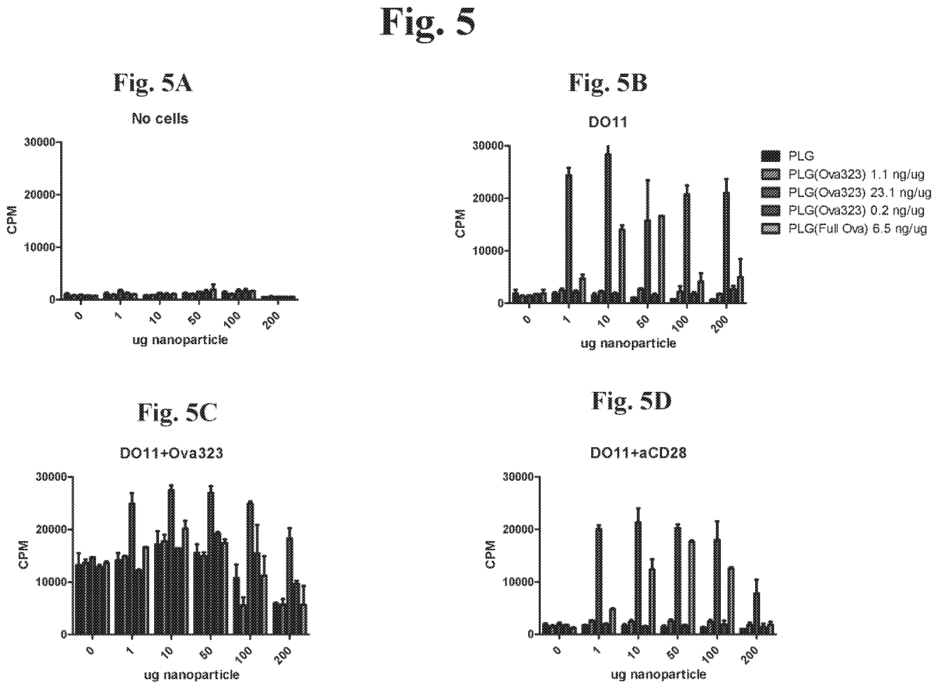

Similar experiments were performed using nanoparticles encapsulating different amounts of Ova₃₂₃₋₃₃₉ (1.1 ng/μg, 23.1 ng/μg, 0.2 ng/μg) or nanoparticles encapsulating full length Ova (FIG. 5). In the absence of addition of the Ova₃₂₃ peptide to the culture, only cells incubated with the nanoparticles encapsulating the greatest amount of Ova₃₂₃₋₃₃₉ peptide (23.1 ng/μg) or full length Ova proliferated (FIG. 5B). Further, the nanoparticles encapsulating the greatest amount of Ova₃₂₃₋₃₃₉ peptide did not inhibit proliferation induced by the addition of Ova323 to the culture at day 2 (FIG. 5C). However, higher doses of nanoparticles encapsulating lower amounts of Ova₃₂₃₋₃₃₉ (1.1 ng/μg and 0.2 ng/μg) reduced the level of cell proliferation induced by Ova323 (FIG. 5C). Addition of αCD28 to the cultures did not significantly affect cell proliferation, indicating that the proliferation in response to Ova323 was antigen-specific. These results indicate nanoparticles encapsulating peptide epitopes can modulate antigen-induced cell proliferation.

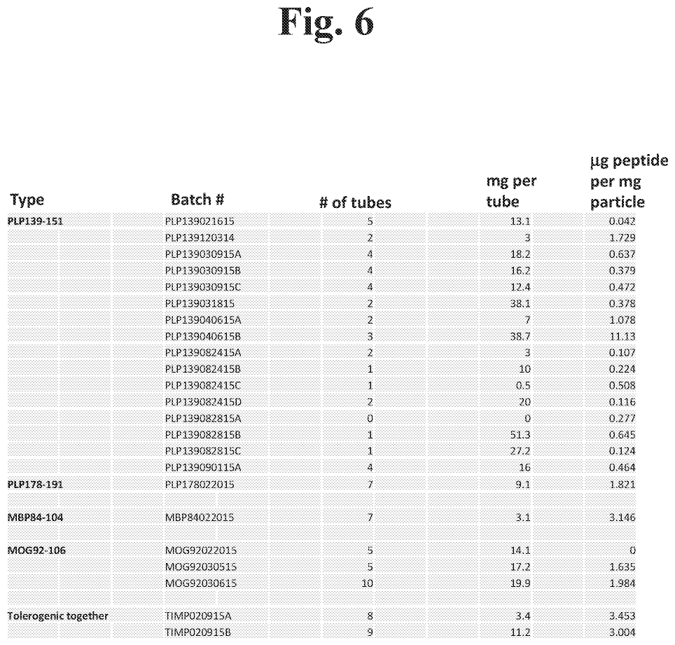

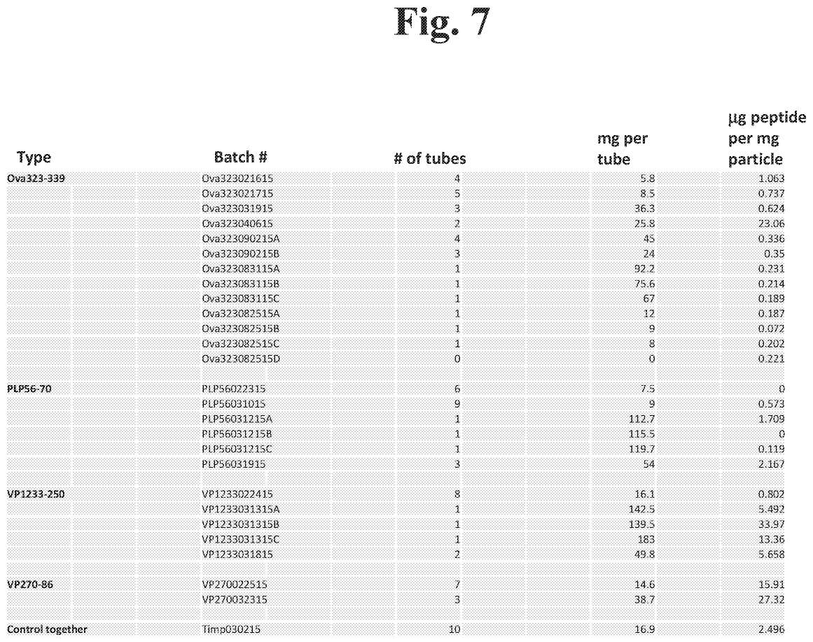

Further, a variety of peptide epitopes can be encapsulated in nanoparticles. For example, FIG. 6 and FIG. 7 show the encapsulation efficiency of tolerogenic and control peptide epitopes, respectively. The peptide epitopes may be encapsulated individually or together. Batches were aliquoted into pre-weighed tubes, dried and weighed to determine the mg/tube of particles. mass in pre-weighed tubes. μg peptide/mg particle was determined using a 3-(4-carboxybenzoyl)quinoline-2-carboxaldehyde (CBQCA) protein quantification assay.

### Example 2—Nanoparticles Encapsulating PLP₁₃₉₋₁₅₁ Peptide Epitopes Modulate Type 1 T Regulatory Cell Populations

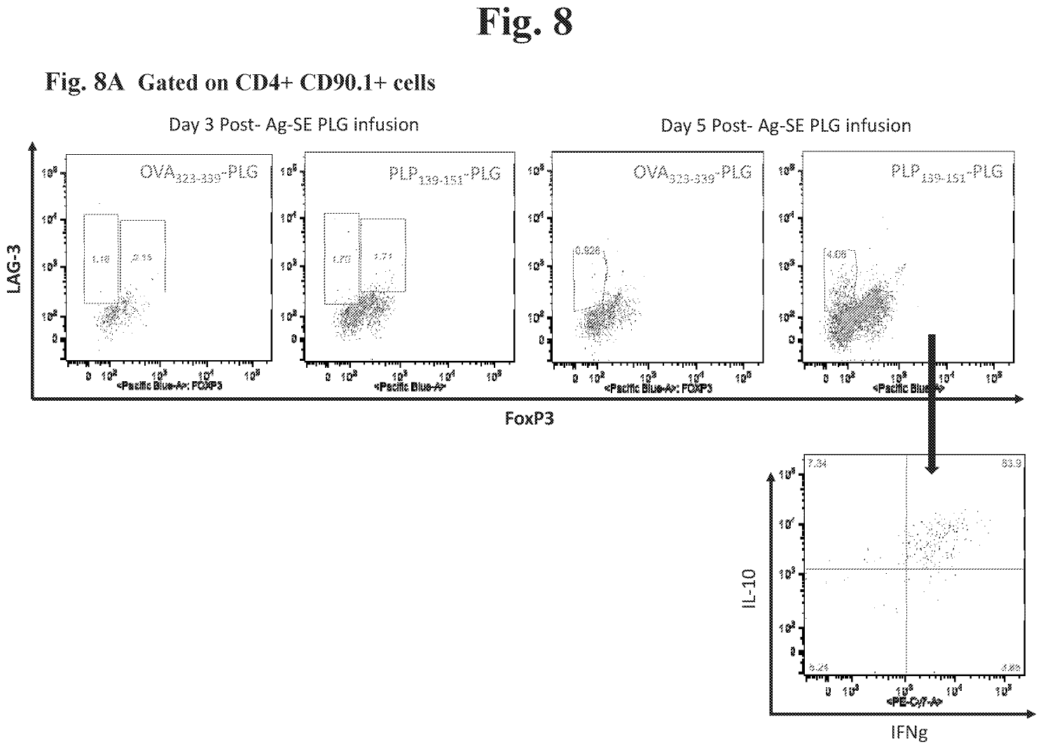

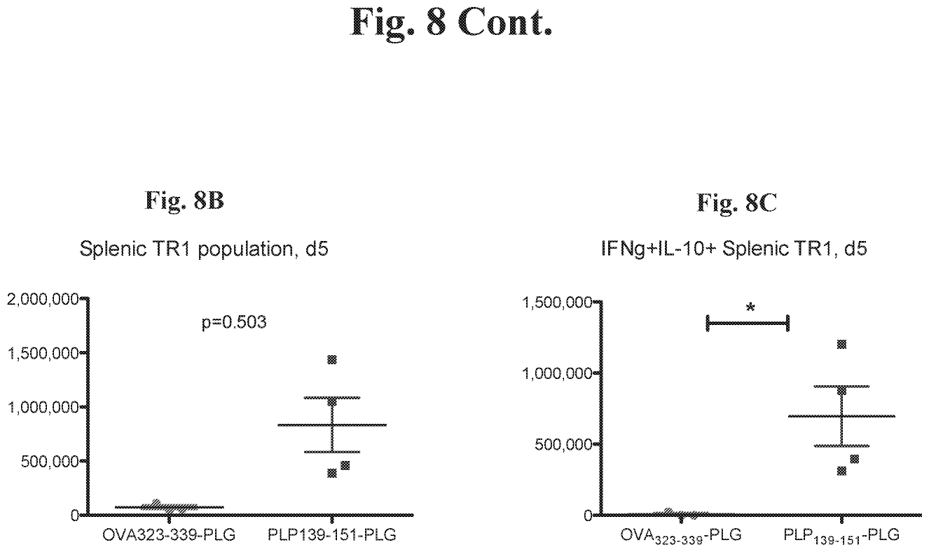

Experiments were performed to assess the effects of nanoparticles encapsulating peptide epitopes on different cell populations using ex vivo assays. Briefly, 3.5×10⁶ CD4⁺ cells from PLP₁₃₉₋₁₅₁ TCR transgenic mice (5B6 mice, donors, Thy1.1⁺) were transferred intravenously into näive, 6-8 week old female SJL recipient mice on day −2. On day 0, SJL recipient mice were infused with either PLG nanoparticles encapsulating PLP₁₃₉₋₁₅₁ SE or PLG nanoparticles encapsulating OVA₃₂₃₋₃₃₉-SE (control particles). Spleens were harvested from recipient mice 3 and 5 days post-infusion, and regulatory T Cell populations were analyzed by flow cytometry (FIG. 8). All populations were gated on CD90.1/Thy1.1⁺ cells to select for the PLP₁₃₉₋₁₅₁-TCR⁺ populations.

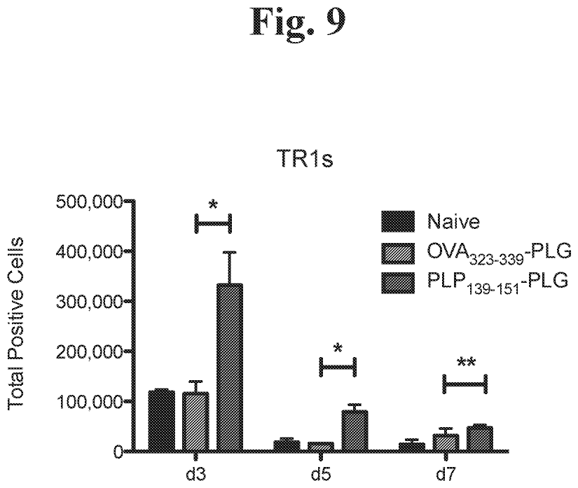

Percentages of Type 1 regulatory T cells (TR1) were determined by gating first for Lag3⁺FoxP3⁻ cells in mice injected with OVA₃₂₃₋₃₃₉-SE PLG or PLP₁₃₉₋₁₅₁-SE PLG on Day 3 (FIG. 8A, left panels) and Day 5 (FIG. 8A, right panels). No difference in the Lag3⁺FoxP3⁻ population was observed at Day 3. However, there was a clear expansion of the Lag3⁺FoxP3⁻ population by Day 5 in the PLP₁₃₉₋₁₅₁-PLG infused animals relative to OVA₃₂₃₋₃₃₉-SE PLG controls. These cells are also largely IFNγ⁺IL-10⁺, indicating that they have a TR1 phenotype (FIG. 8A, bottom panel). Quantification of the number of Lag3⁺FoxP3⁻ cells showed no difference between PLP₁₃₉₋₁₅₁-PLG treated and OVA₃₂₃₋₃₃₉-SE PLG-treated mice. However, PLP₁₃₉₋₁₅₁-PLG treated mice showed a significant increase in the number of antigen-specific TR1 cells (LAG3⁺FoxP3⁻IFNγ⁺IL-10⁺) compared to OVA₃₂₃₋₃₃₉-SE PLG-treated controls. Similar experiments were done with spleens harvested at day 3, 5, and 7 post-infusion of PLP₁₃₉₋₁₅₁-SE PLG or OVA₃₂₃₋₃₃₉-SE PLG (FIG. 9). Similar to the results in FIG. 8, the number of TR1 cells was significantly increased in mice treated with PLP₁₃₉₋₁₅₁-PLG. These data demonstrate that PLG nanoparticles encapsulating peptide epitopes increase the number of regulatory T cells, indicating a potential therapeutic role in tolerance induction.

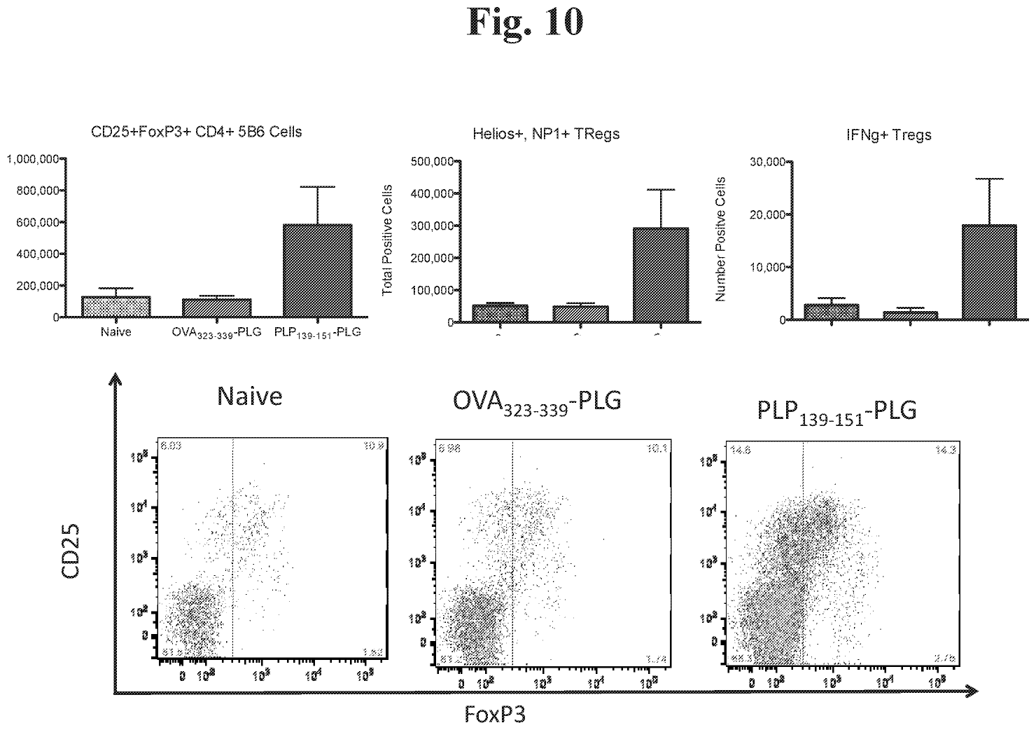

Separate experiments were performed according to the same protocol and splenic regulatory T cells were analyzed by flow cytometry. Cells were gated on CD90.1/Thy1.1 (PLP₁₃₉₋₁₅₁TCR⁺) populations as above and analyzed for expression of the regulatory T cell markers CD25, FoxP3, Helios, NP1, and IFNγ. As shown in FIG. 10, infusion of PLP₁₃₉₋₁₅₁-PLG increased the number of CD25⁺FoxP3⁺, Helios⁺NP1⁺, and IFNγ⁺ regulatory T cell populations. These results further support the conclusion that PLG nanoparticles encapsulating peptide epitopes have a potential therapeutic role in tolerance induction.

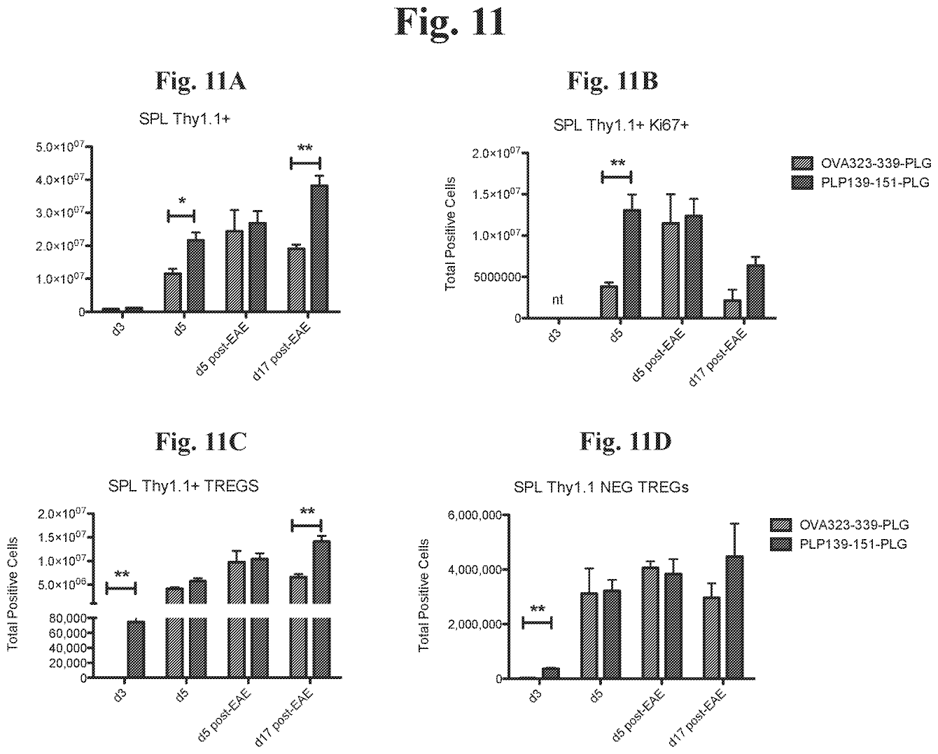

The ability of PLG nanoparticles encapsulating peptide epitopes to modulate disease activity was tested in EAE, a mouse model of multiple sclerosis. Briefly, 3.5×10⁶ CD4⁺ cells from 5B6 donors were transferred intravenously into naïve, 6-8 week old, female SJL mice on day −2. On Day 0, SJL recipient mice were infused with either PLP₁₃₉₋₁₅₁-SE PLG or control OVA₃₂₃₋₃₃₉-SE PLG. Spleens were harvested from mice on days +3 and +5 relative to infusion. EAE was induced in a separate cohort on day 7, and spleens were harvested from these mice for analysis on days 5 and 17 after EAE induction. Splenic T cell populations were then analyzed by flow cytometry. Cell populations were gated on CD90.1/Thy1.1 (PLP₁₃₉₋₁₅₁TCR⁺) populations (FIG. 11A). As shown, infusion with PLP₁₃₉₋₁₅₁-SE PLG significantly increased the number of antigen-specific T cells compared to OVA₃₂₃₋₃₃₉-SE PLG-infused controls at later time point in both EAE and non-EAE mice (FIG. 11A). Further, infusion with PLP₁₃₉₋₁₅₁-SE PLG significantly increased the number of proliferating antigen-specific T cells (FIG. 11B) and the number of antigen-specific regulatory T cells compared to OVA₃₂₃₋₃₃₉-SE PLG-infused controls (FIG. 11C). There was no significant difference in the number of non-antigen specific regulatory T cells (FIG. 11D). These results indicate that PLP₁₃₉₋₁₅₁-SE PLG resulted in the increased proliferation of antigen-specific T regulatory cells, indicating that nanoparticles encapsulating peptide epitopes to mediate tolerogenic responses in models of autoimmunity.

### Example 3—Nanoparticles Encapsulating OVA₃₂₃₋₃₃₉ Peptide Epitopes Modulate Type 1 T Regulatory Cell Populations

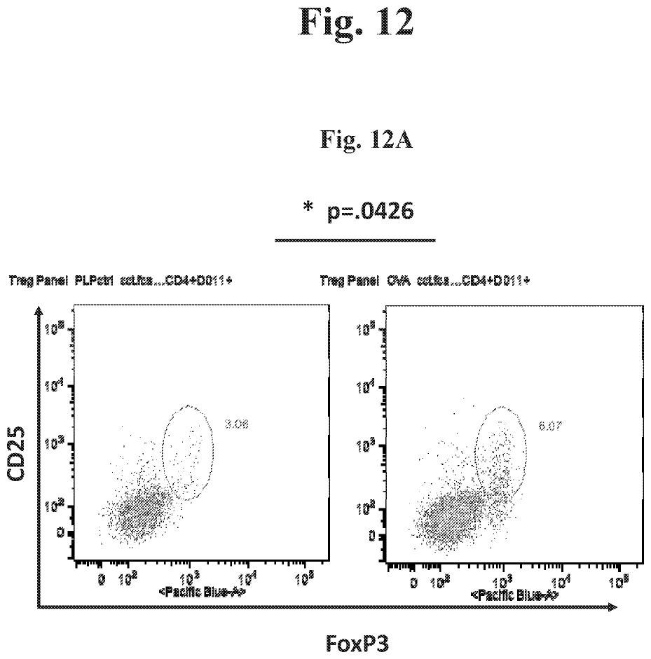

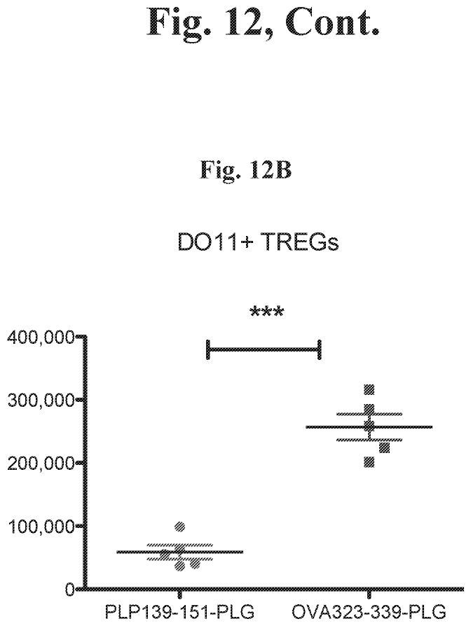

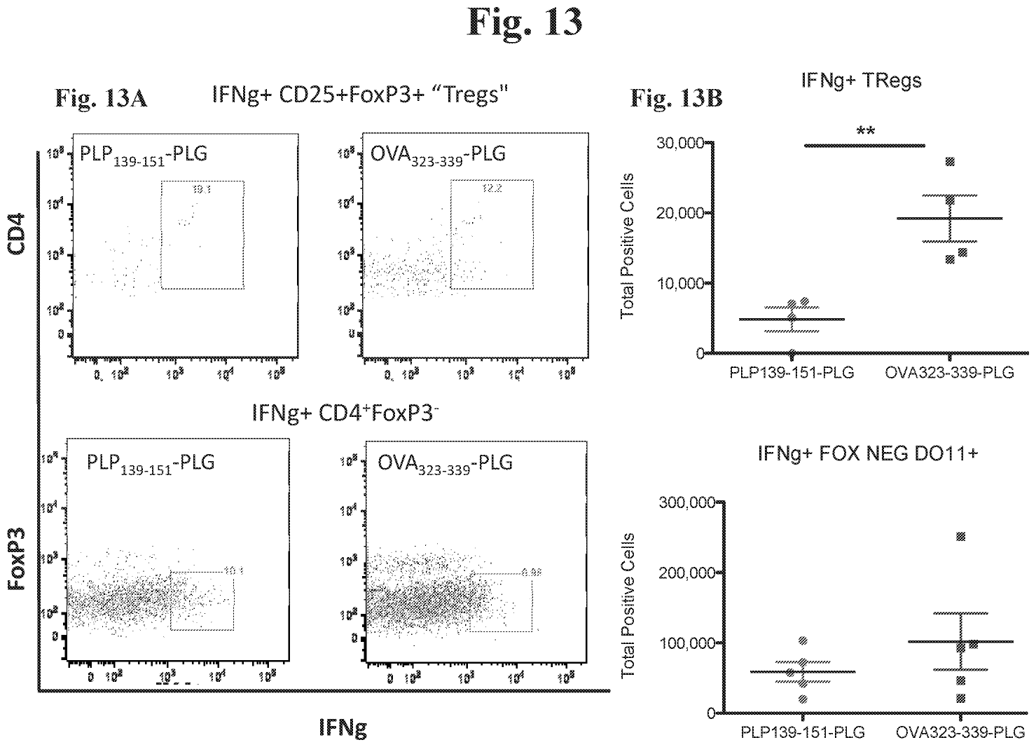

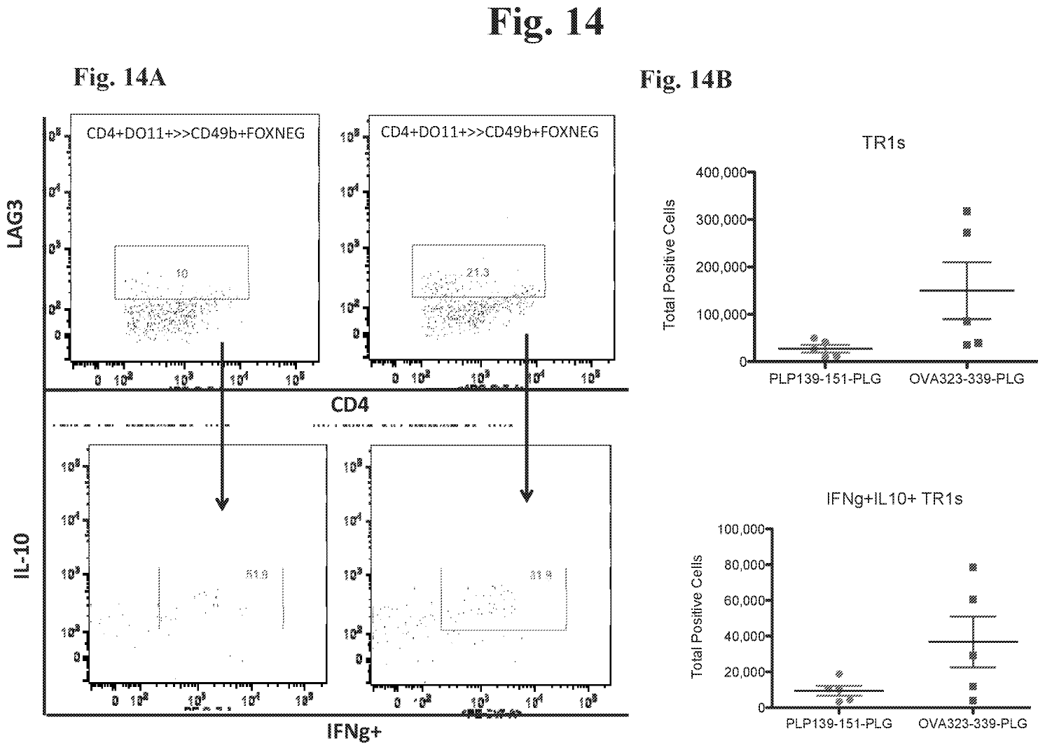

Complementary experiments were performed, wherein 2.5×10⁶ CD4⁺ cells from OVA₃₂₃₋₃₃₉ TCR donors (DO11 mice) were transferred intravenously into naïve female Balb/c RAG KO recipient mice, aged 6-8 weeks. Two weeks after transfer, recipient mice were infused with either OVA₃₂₃₋₃₃₉-SE PLG or PLP₁₃₉₋₁₅₁-SE PLG (control). Spleens were harvested from mice on day post-infusion, and regulatory T Cell populations were analyzed by flow cytometry (FIG. 12). Cell populations were on DO11-TCR⁺ populations. As shown, there was a significant increase in both the percentage (FIG. 12A) and total number (FIG. 12B) of CD25⁺FoxP3⁺CD4⁺ T cells from OVA₃₂₃₋₃₃₉-PLG-infused animals relative to PLP₁₃₉₋₁₅₁-SE PLG-infused controls. In similar experiments, the percentage (FIG. 13A) and number (FIG. 13B) of IFNγ-producing antigen-specific regulatory T cells was also significantly increased in OVA₃₂₃₋₃₃₉-PLG-infused animals relative to PLP₁₃₉₋₁₅₁-SE PLG-infused controls. There was no significant difference in the number of IFNγ⁺ non-regulatory T cells between experimental and control groups (FIG. 13). Further, there was a trend toward increased percentages (FIG. 14A) and numbers (FIG. 14B) of TR1 populations from DO11 donors, similar to the trend seen with the 5B6 CD90.1⁺TR1 cells isolated from SJL mice.

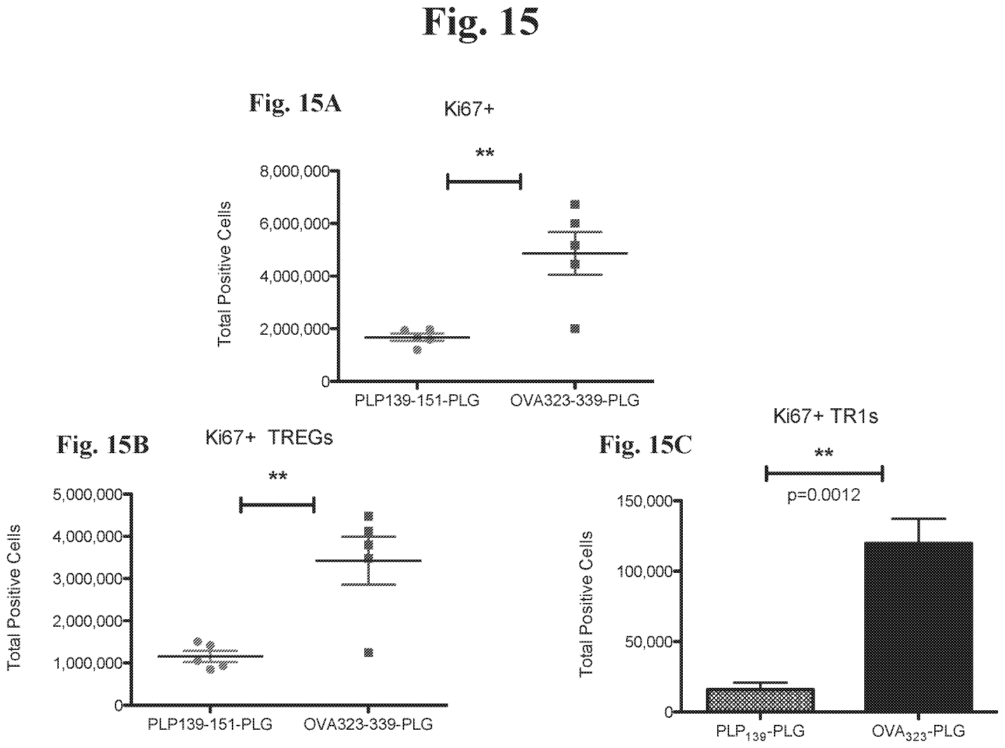

There was also an increase in the number of proliferating antigen-specific cells (FIG. 15A), the number of proliferating antigen-specific T regulatory cells (FIG. 15B) and the number of proliferating antigen specific TR1 cells (FIG. 15C, from a separate experiment, not confirmed as FoxP3⁻). These results indicate that, similar to the 5B6-transfer experiments in Example 2, PLG nanoparticles encapsulating peptide epitopes regulate the expansion of T regulatory cell populations, indicating a role in the induction of tolerance.

### Example 4—Characterization of Nanoparticles Encapsulating PLP139-Ova323 Fusion Peptides

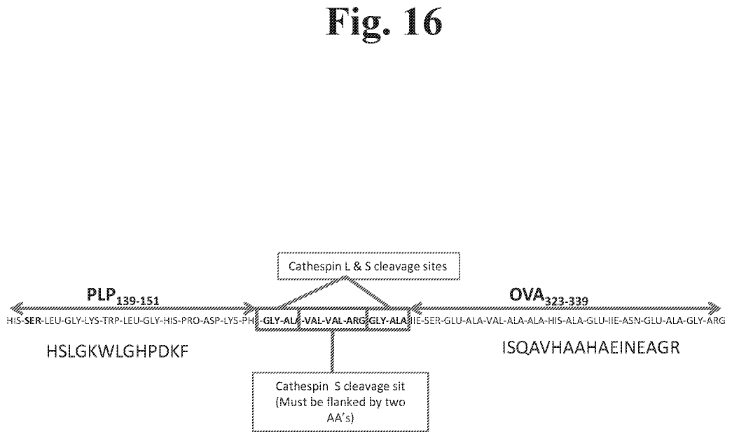

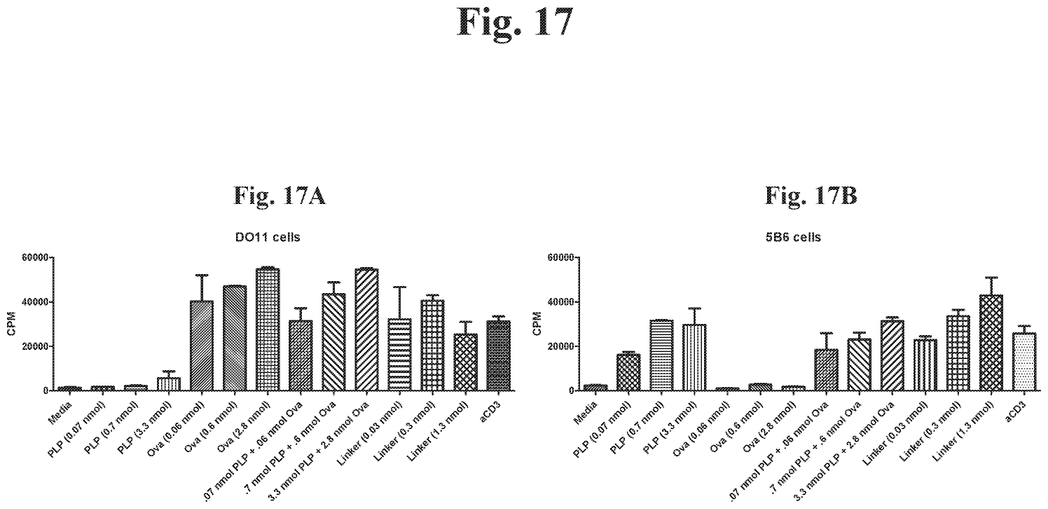

Fusion peptides of the PLP₁₃₉₋₁₅₁ and OVA₃₂₃₋₃₃₉ epitopes linked by a peptide linker comprising a cathepsin-specific cleavage site were generated (FIG. 16, PLP139-Ova323 fusion peptide). 2×10⁵ splenocytes from DO11.10 (FIG. 17A) or 5B6 (FIG. 17B) transgenic mice were plated with varying amounts of OVA₃₂₃₋₃₃₉, PLP₁₃₉₋₁₅₁, OVA₃₂₃₋₃₃₉+PLP₁₃₉₋₁₅₁, or PLP139-Ova323 fusion peptide (linker) and cell proliferation was assessed. The PLP139-Ova323 fusion peptide induced cell proliferation that was comparable to the responses induced by OVA₃₂₃₋₃₃₉ in DO11 cells and PLP₁₃₉₋₁₅₁ in 5B6 cells. These results indicate that the fusion protein is capable of modulating cellular responses, similar to either epitope alone or in combination.

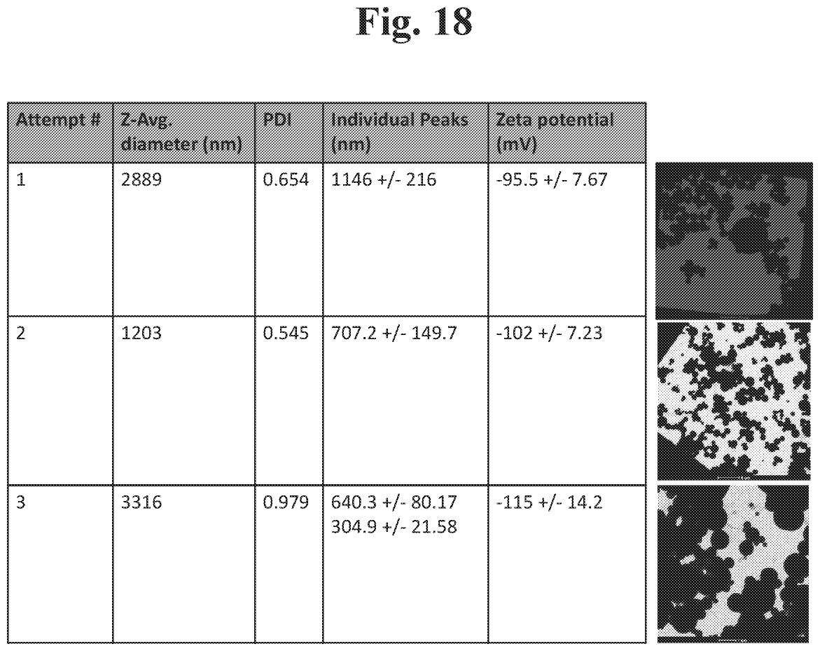

The physical characteristics (Z-Avg diameter, PDI, Peak diameter, and zeta potential) of nanoparticles encapsulating the PLP139-Ova323 fusion peptide were determined by dynamic light scattering (DLS) (FIG. 18). The Z-Avg diameter ranged from 1203-3316 nm over three attempts and the zeta potential was between −95 and −115 mV. Images of the nanoparticles were also obtained using transmission electron microscopy (TEM) with particles dried on carbon-coated copper mesh grids (FIG. 18).

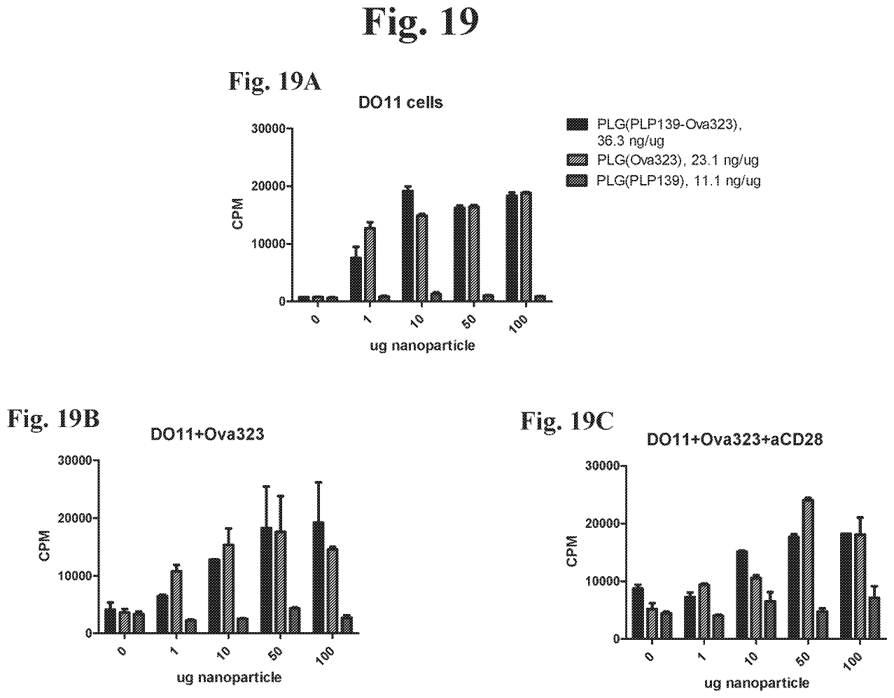

The effects of the nanoparticles on cell proliferation were assessed by plating 2×10⁵ DO11 splenocytes with 1-100 μg of nanoparticles (nanoparticles encapsulating PLP139-Ova323 fusion, PLP₁₃₉₋₁₅₁, or OVA₃₂₃₋₃₃₉). Cells were allowed to grow for 2 days and were then pulsed with thymidine and cultured for an additional 3 days. Treatment of cells with either PLP139-Ova323 fusion or OVA₃₂₃₋₃₃₉ encapsulated nanoparticles resulted in increased cellular proliferation compared to untreated controls (FIG. 19A). Similar results were observed when 1 μg Ova₃₂₃ (FIG. 19B) or 1 μg Ova₃₂₃ and 1 μg/mL αCD28 (FIG. 19C) were added to the culture on Day 2.

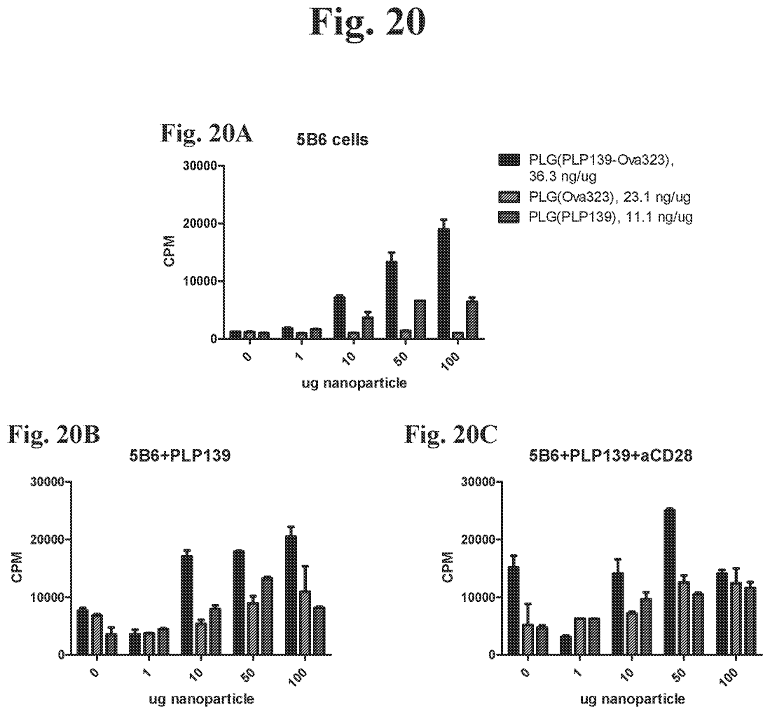

Complementary experiments were performed with 5B6 splenocytes (FIG. 20). 2×10⁵ 5B6 splenocytes were plated and allowed to grow for 2 days, then pulsed with thymidine and cultured for an additional 3 days. Treatment of cells with nanoparticles encapsulating either PLP139-Ova323 fusion or PLP₁₃₉₋₁₅₁ resulted in increased cell proliferation at higher doses, compared to untreated controls or cells treated with nanoparticles encapsulating OVA₃₂₃₋₃₃₉ (FIG. 20A). Similar results were observed when 1 μg PLP139 (FIG. 20B) or 1 μg PLP139 and 1 μg/mL αCD28 (FIG. 20C) were added to the culture on Day 2. These data demonstrate that the linked-epitope protein fusions are immunogenic and suggest that they have increased immune modulating potential compared to the single epitopes alone.

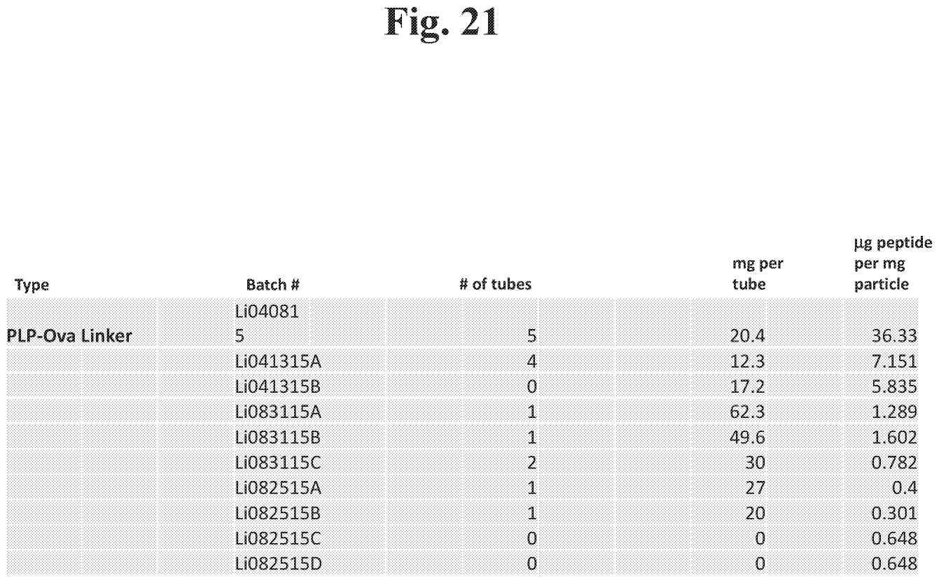

The fusion protein can be efficiently encapsulated in a nanoparticle, as shown in FIG. 21. Batches were aliquoted into pre-weighed tubes, dried and weighed to determine the mg/tube of particles. mass in pre-weighed tubes. μg peptide/mg particle was determined using a CBQCA protein quantification assay.

### Example 5—Nanoparticles Encapsulating PLP139-Ova323 Fusion Peptides Mediate Tolerogenic Responses in EAE

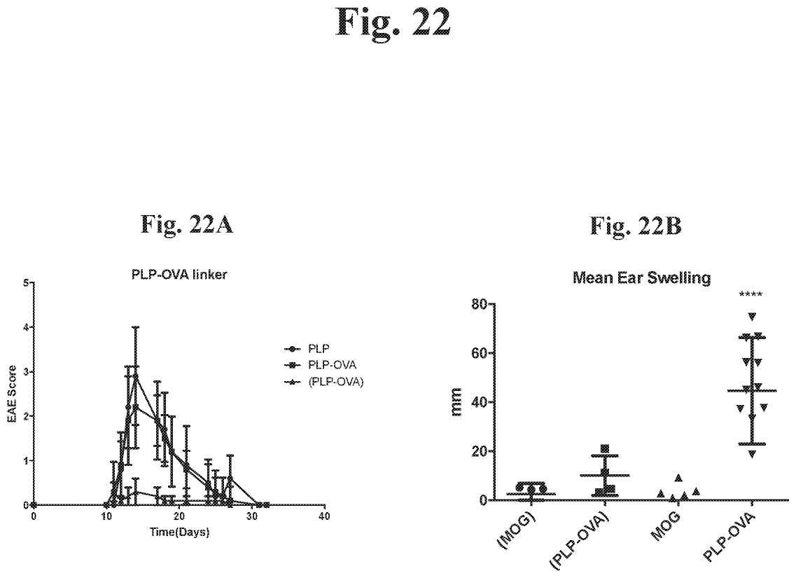

Experiments were performed to determine the in vivo effects of nanoparticles encapsulating PLP139-OVA323 fusion protein in a model of EAE. EAE was induced in SLJ mice by immunization with the PLP₁₃₉₋₁₅₁ peptide epitope or PLP139-OVA323 fusion protein emulsified in adjuvant (N=5 per group). As shown in FIG. 22A, immunization with the PLP139-OVA323 fusion protein was sufficient to induce EAE. The magnitude of the resultant disease score was comparable to that induced by PLP₁₃₉₋₁₅₁ peptide immunization (FIG. 22A). FIG. 22→PLP-OVA linked EAE scores. PLP-OVA linked peptide induces disease and induces tolerance when encapsulated in PLGA nanoparticles. Importantly, administration of encapsulated PLP-OVA fusion protein (PLP-OVA) resulted in abrogation of EAE disease score (FIG. 22A). Further, this immune-modulation was antigen specific, as administration of encapsulated PLP-OVA fusion protein only reduced immune responses in SLJ mice immunized with the PLP, and did not affect immune responses induced by MOG peptide immunization (FIG. 22B). These results indicate that nanoparticles encapsulating linked-epitope fusion peptides induce tolerogenic immune responses in an antigen-specific manner.

### Example 6—Characterization of Nanoparticles Encapsulating Multi-Epitope Fusion Peptides

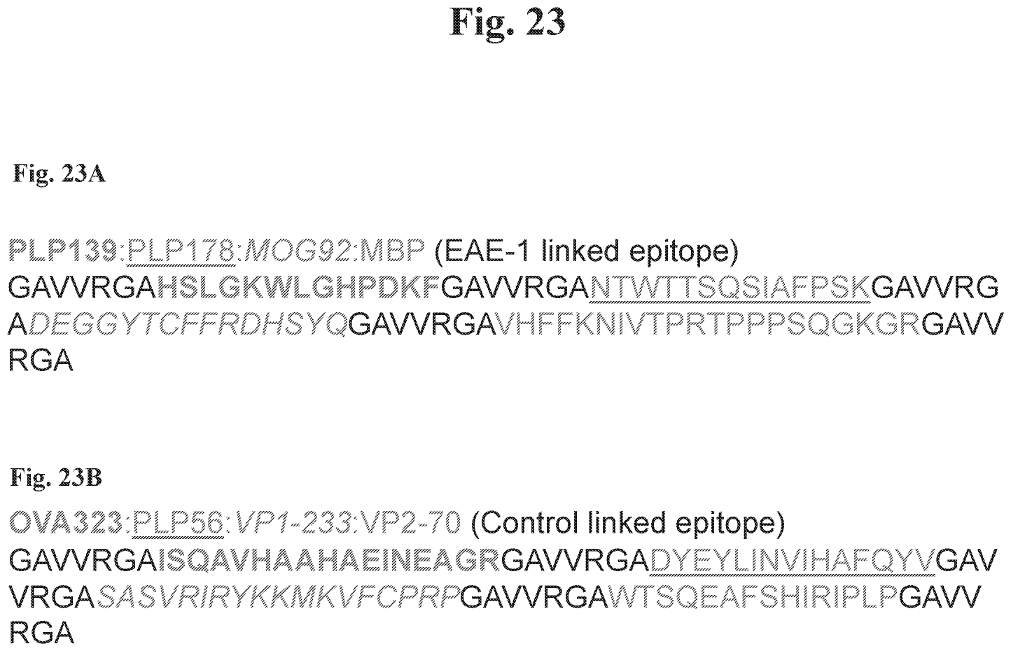

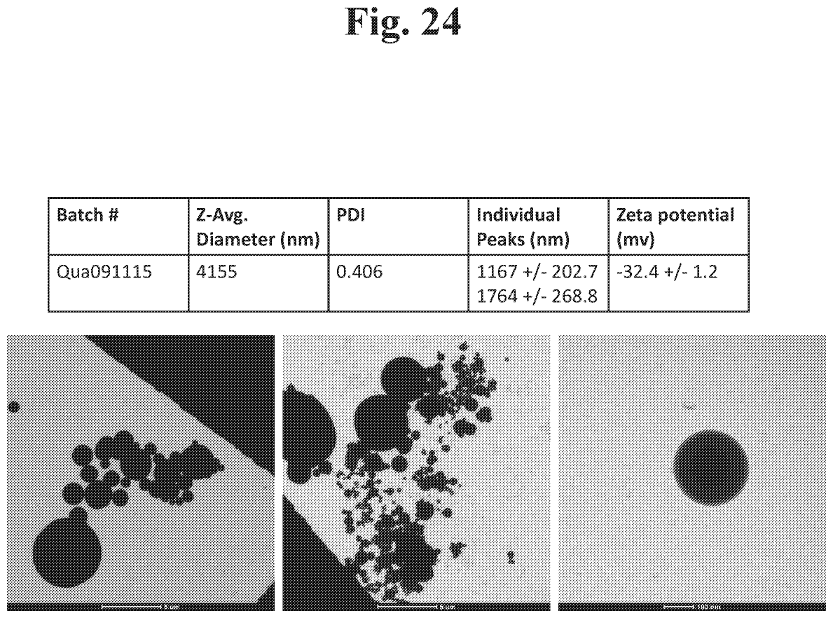

Fusion peptides of the PLP₁₃₉₋₁₅₁, PLP₁₇₈₋₁₉₁, MOG₉₂₋₁₀₆, and MBP₆₄₋₁₀₄ epitopes linked by a peptide linker comprising a cathepsin-specific cleavage site were generated (FIG. 23A, tolerogenic EAE-1 fusion peptide). Additionally, control fusion peptides comprising OVA₃₂₃₋₃₃₉, PLP₅₆₋₇₀, VP1₂₃₃₋₂₅₀, and VP2₇₀₈₆ epitopes linked by a peptide linker comprising a cathepsin-specific cleavage site were generated (FIG. 23B, EAE-1 control fusion peptide). Physical characteristics (Z-avg diameter, PDI, Peak diameter, and zeta potential) of nanoparticles encapsulating the tolerogenic fusion peptide were determined by DLS. Images of the particles were obtained using transmission electron microscopy (TEM) with particles dried on carbon-coated copper mesh grids. Both DLS and EM data indicate larger than normal size distribution (FIG. 24).

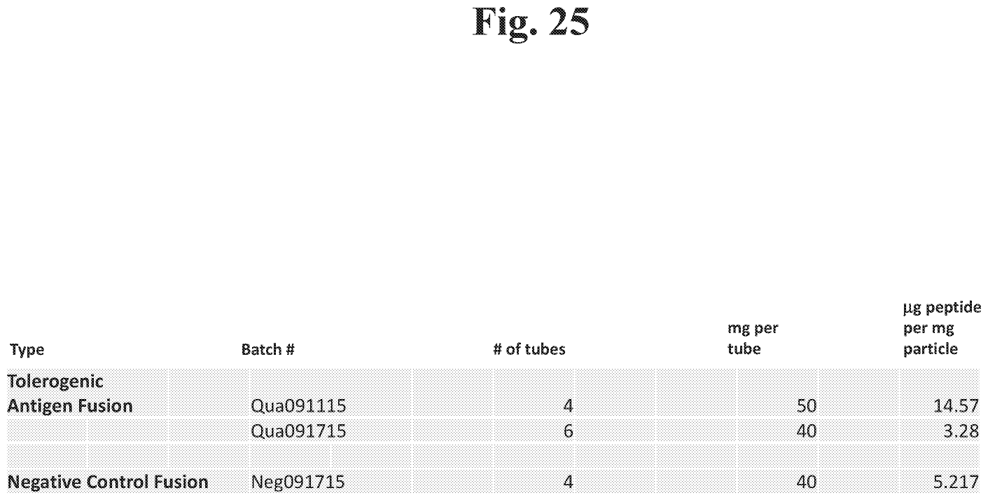

Encapsulation efficiency for tolerogenic fusion peptide or negative control fusion peptides are shown in FIG. 25. Batches were aliquoted into pre-weighed tubes, dried and weighed to determine the mg/tube of particles. mass in pre-weighed tubes. μg peptide/mg particle was determined using a CBQCA protein quantification assay.

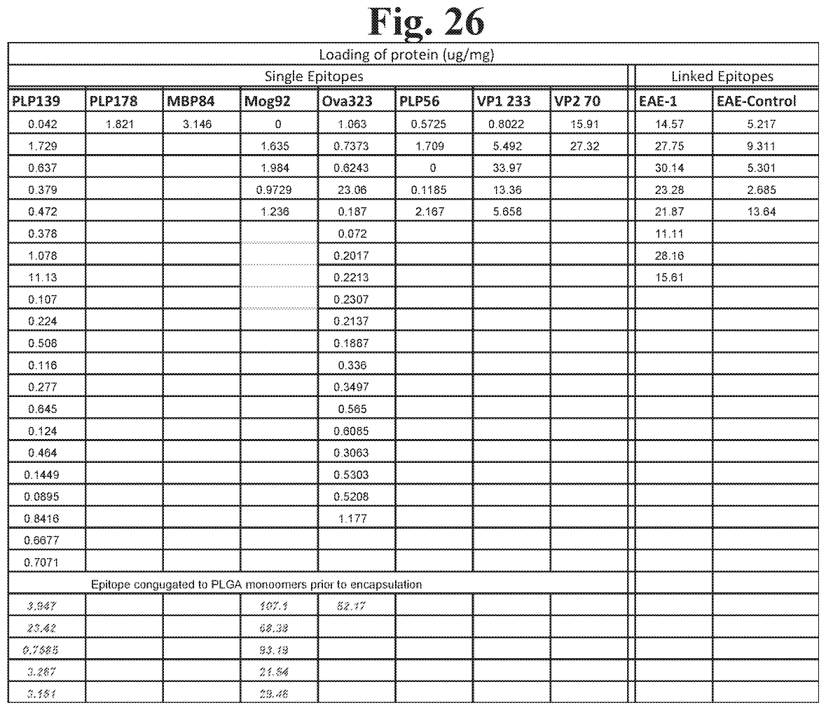

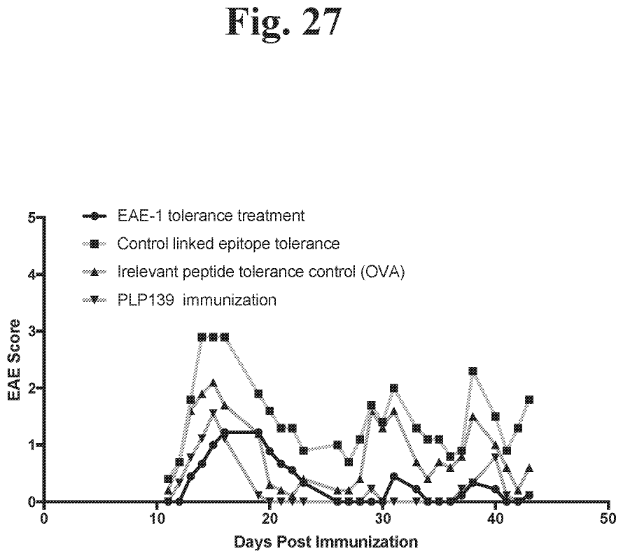

Encapsulation of single and linked epitopes into PLGA nanoparticles is shown in FIG. 26. Single myelin specific T cells epitopes (PLP139, PLP178, MBP84, and MOG92) were encapsulated at lower efficiencies than linked EAE-1 peptides (PLP139:PLP178:MOG92:MBP), as determined by one way-ANOVA statistical test (p<0.0001). Similarly, individual control epitopes (Ova323, PLP56, VP1233 and VP270) were encapsulated at lower efficiencies than linked EAE-control epitopes (OVA323: PLP56: VP1: VP2).

### Example 7—Characterization of Nanoparticles Encapsulating Multi-Epitope Fusion Peptides

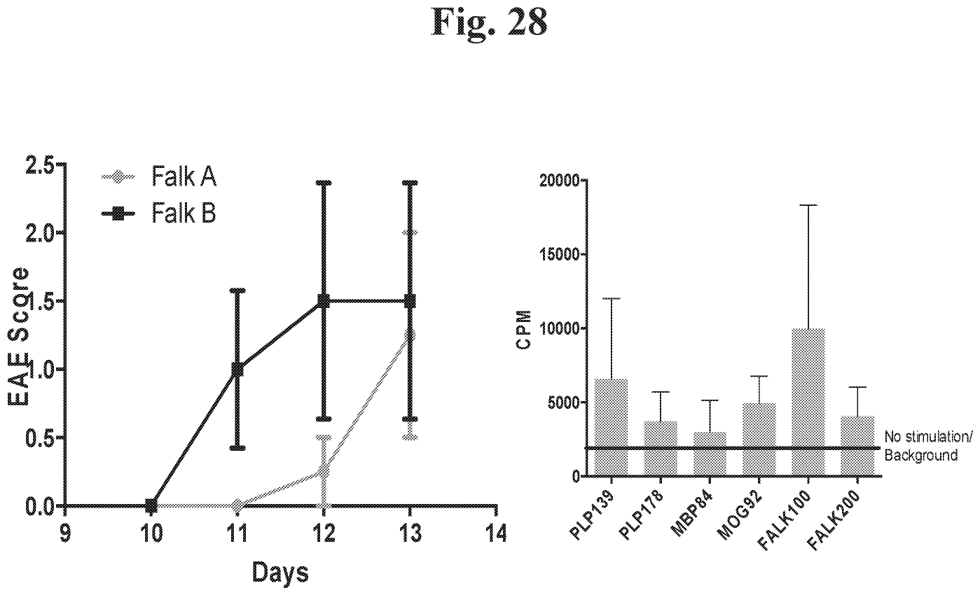

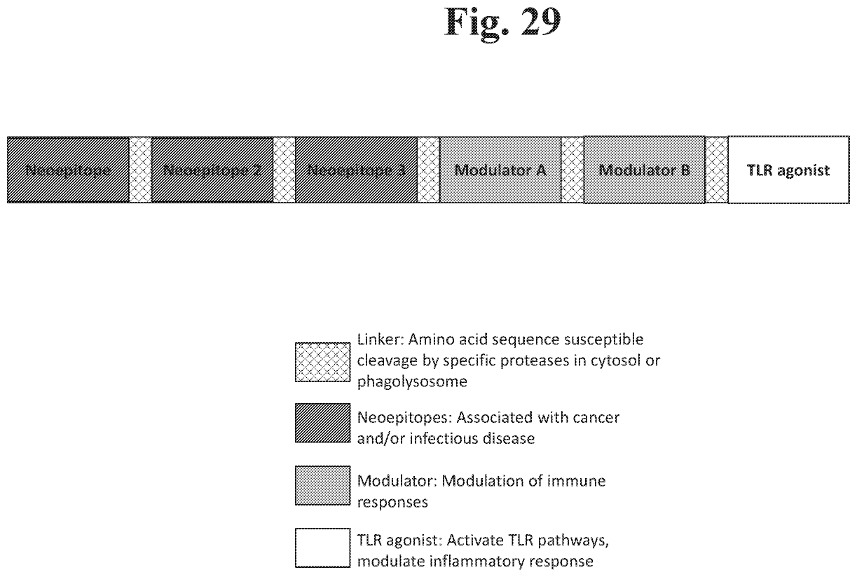

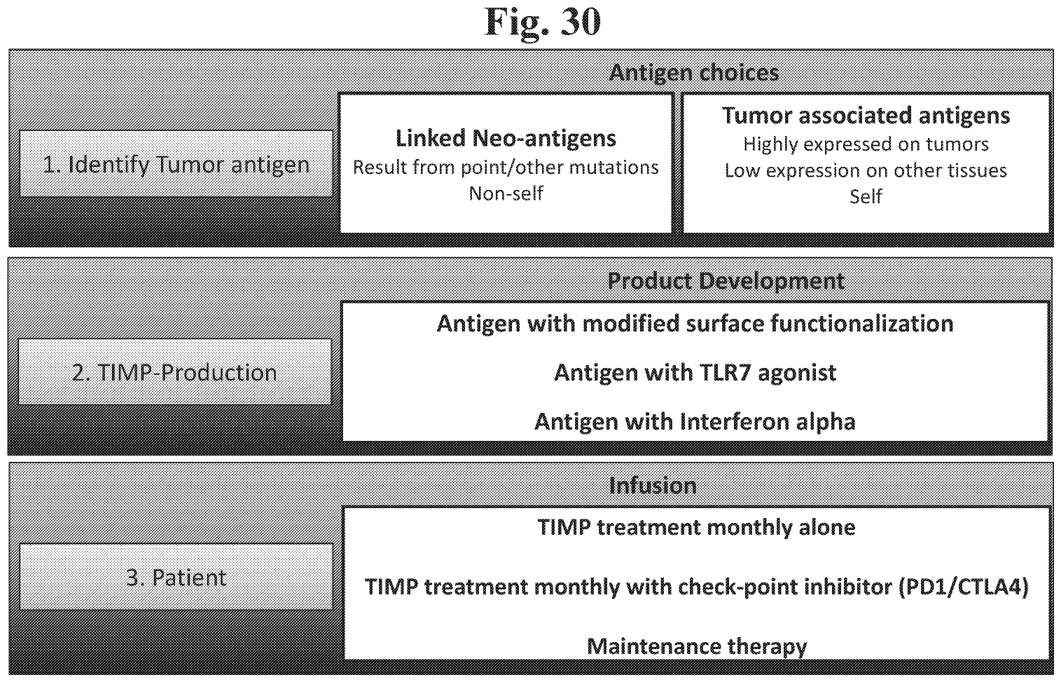

Experiments were performed to determine the effects of PLGA nanoparticles encapsulating the linked EAE-1 fusion peptide (PLP139:PLP178:MOG92:MBP) in an EAE model into nanoparticles results in significant reduction in EAE disease, compared to irrelevant control peptide (OVA) or a control linked epitope (OVA323:PLP56:VP1:VP2). Treatment of mice with encapsulated linked EAE-1 fusion peptide significantly reduced EAE disease score compared to treatment with the control fusion peptide or irrelevant control peptide (OVA) (FIG. 28). These data demonstrate that fusion peptides comprising multiple linked disease epitopes can induce tolerance in models of autoimmunity and indicate their therapeutic potential. FIG. 29 demonstrates that FALK peptides can also induce EAE, indicating that epitopes found in this protein may be incorporated into tolerogenic EAE fusion proteins as well.

### Example 8—Use of Encapsulated Linked Epitope Fusion Proteins in Treatment of Cancer

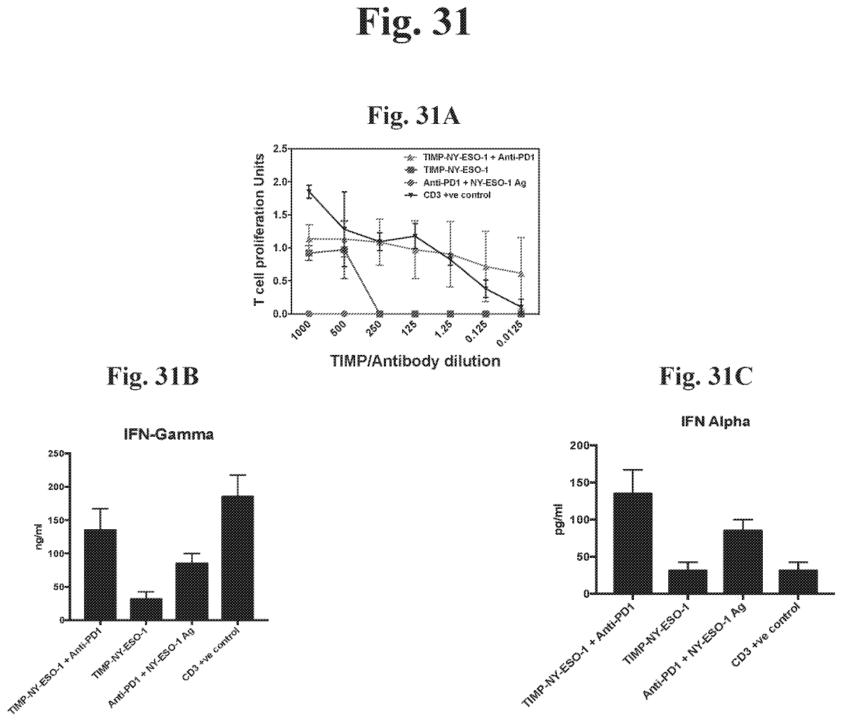

Fusion peptides comprising linked epitopes of neoantigens or tumor antigens are also used in the treatment of cancer. Such epitopes are combined with immune modulators, such as PD1, or Toll-like receptor agonists to increase therapeutic efficacy (See, FIG. 29). Data will show that encapsulated cancer antigens, such as Ny-Esol, delivered with an immune modulator such as anti-PD1 will result in T cell proliferation (See exemplary results in FIG. 31A), IFNγ production (See exemplary results in FIG. 31B), and IFNα production (See exemplary results in FIG. 31C). These results will show that encapsulated cancer antigen mediate immunogenic responses, resulting in pro-inflammatory cytokine production.

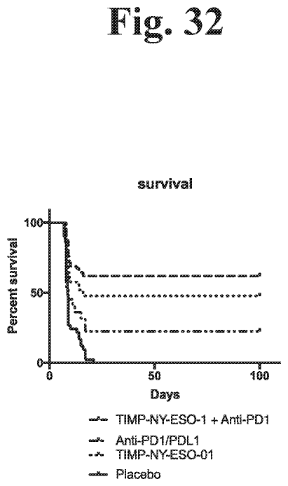

Further, treatment with encapsulated Ny-Esol will result in increased survival of mice in a murine model of melanoma. 0.01 mg/Kg infusions of encapsulated NY-ESO-1 (TIMP-NY-ESO-1) are given twice monthly (7 days apart) 6 months. Inhibition of PD1/PD-L 1 is achieved with Pembrolizumab or Nivolumab or Atezolizumab. TIMP-NY-ESO-1 treatment alone is sufficient to increase survival time and the effect is enhanced when was combined with anti-PD1 treatment (See exemplary results in FIG. 32).

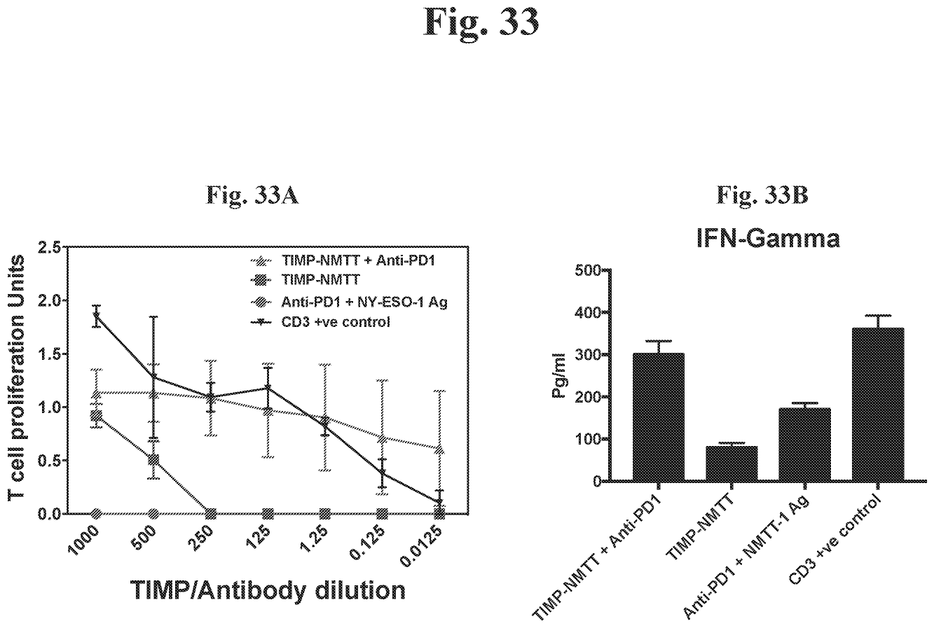

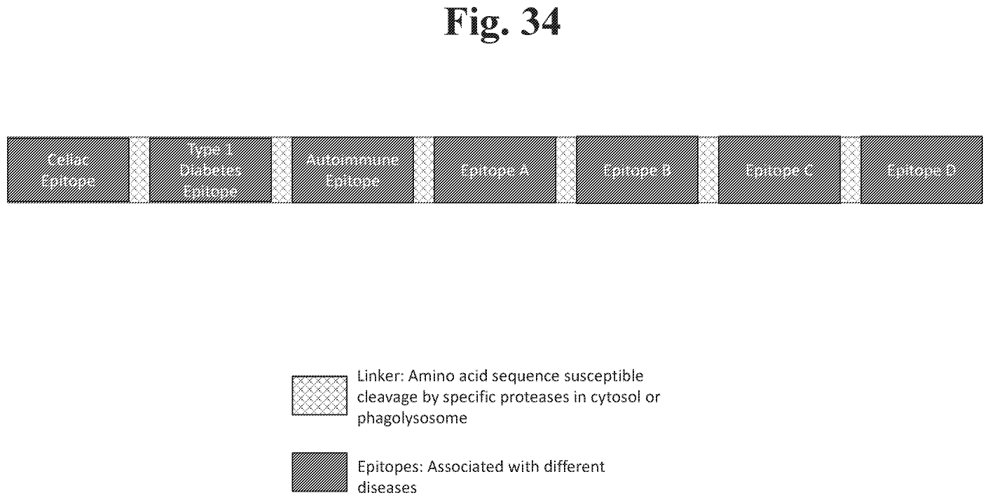

Fusion peptides comprising linked cancer epitopes (NY-ESO-1, Mage-A3, TPTE, Tyrosinase, HPU16) have been previously described (See Kranz et al., Nature, V. 534, pp 396-401, 2016; U.S. Patent Pub. No. 2011-0070252). Fusion proteins comprising 4 cancer epitopes (NY-ESO-1, Mage-A3, TPTE, Tyrosinase) are generated to form an NMTT fusion protein. Various concentrations of encapsulated NMTT (TIMP-NMTT) are incubated with peripheral blood monocytes (PBMCs) from healthy subjects with or without anti-PD1/PDL1 treatment Cultures are incubated for 3-5 days and T cell proliferation is determined using cell-tire glow or incorporation of tritiated thymidine. IFNγ concentrations are determined using an ELISA. Incubation of PBMCs with TIMP-NMTT in combination with anti-PD1/PDL1 will result in increased T cell proliferation compared to controls (Exemplary results shown in FIG. 33A). Further, TIMP-NMTT in combination with anti-PD1/PDL1 will result in IFNγ production from PBMCs, whereas TIMP-NY-ESO-1 alone will have a negligible impact. These results will show that encapsulated fusion protein of cancer epitopes mediate immunogenic responses that are beneficial in the context of cancer treatment.

To determine the clinical effects of TIMP-NMTT, tumor antigen encoding TIMP-NMTT are prepared from GMP-manufactured components in a dedicated pharmacy under GMP. Patients are injected i.v. with weekly escalating doses of TIMP-NMTT encoding antigens NY-ESO-149, tyrosinase 50, MAGE-A351 and TPTE52 (1.9, 3.6 or 7.2 μg TIMP-NMTT of each antigen). Blood samples are obtained for cytokine measurements prior to vaccination, 2, 6, and 24 hours after vaccination on day 1, and on days 8 and 15 after vaccination for ELISPOT and MHC class I dextramer staining analyses. Blood samples for T-cell monitoring are obtained before vaccination on the respective vaccination day. Clinically administered TIMP-NMTT vaccines will dose-dependently induce systemic INFα and de novo T cell response.

### Example 9—Additional Uses of Encapsulated Linked Epitope Fusion Proteins

Additional fusion proteins comprising linked epitopes may be generated. For example, fusion proteins comprising epitopes derived from multiple disease types may be generated and used in the treatment of more than one disease. Alternatively, fusion proteins comprising disease-related epitopes may be generated that further comprise a TLR agonist. Inclusion of such an agonist will increase the immunogenicity of the protein and increase the therapeutic efficacy. Further fusion proteins may be generated with epitopes derived from infectious viruses, bacteria, or fungi. Addition of a TLR agonist to such constructs may also increase the therapeutic efficacy.

1. A biodegradable particle comprising one or more fusion proteins encapsulated therein;
wherein each one of said one or more fusion proteins comprises two or more antigenic epitopes;
wherein said two or more antigenic epitopes are separated by a linker; wherein the linker is cleavable by an intracellular protease;
and
wherein said biodegradable particle has a negative zeta potential between about −100 mV to about 0 mV.

2-6. (canceled)

7. The biodegradable particle of claim 1, wherein said biodegradable particle has a zeta potential of between about −80 mV and about −30 mV.

8-9. (canceled)

10. The biodegradable particle of claim 1, wherein said biodegradable particle has a diameter of between about 0.1 μm to about 10 μm.

11-15. (canceled)

16. The biodegradable particle of claim 1, wherein the amino acid sequence of the linker is susceptible to site-specific cleavage by proteases.

17-26. (canceled)

27. The biodegradable particle of claim 1, wherein the one or more antigenic epitopes comprise autoimmune antigens, antigens expressed on a tissue to be transplanted into a subject, antigens derived from an enzyme for enzyme replacement therapy, or antigens derived from an allergen or combinations thereof.

28-54. (canceled)

55. A pharmaceutical composition comprising a biodegradable particle of claim 1.

56-57. (canceled)

58. A method of inducing antigen-specific tolerance in a subject comprising administering an effective amount of the biodegradable particle of claim 1.

59. A method of inducing antigen-specific tolerance in a subject comprising administering to the subject an effective amount of a biodegradable particle comprising one or more fusion proteins encapsulated therein;
wherein each one of said one or more fusion proteins comprises two or more antigenic epitopes;
wherein said two or more antigenic epitopes are separated by a linker;
and,
wherein said biodegradable particle has a negative zeta potential between about −100 mV and about 0 mV.

60-63. (canceled)

64. The method of claim 59, wherein the effective amount of the biodegradable particle is administered to the subject to treat or prevent a disease or condition.

65. The method of claim 64, wherein the disease or condition is selected from the group consisting of an autoimmune disease, a lysosomal storage disease, an enzyme deficiency, inflammatory disease, an allergy, transplantation rejection, and a hyperimmune response.

66-81. (canceled)

82. A method of decreasing inhibitory neutrophil accumulation in a subject comprising administering to the subject an effective amount of a biodegradable particle comprising one or more fusion proteins encapsulated therein;
wherein each one of said one or more fusion proteins comprises two or more antigenic epitopes;
wherein said two or more antigenic epitopes are separated by a linker;
and,
wherein said biodegradable particle has a negative zeta potential between about −100 mV and about 0 mV.

83. The method of claim 82, wherein the subject has cancer.

84-110. (canceled)

111. A method of treating multiple sclerosis in a subject comprising administering to the subject an effective amount of a biodegradable particle comprising one or more fusion proteins encapsulated therein;
wherein each one of said one or more fusion proteins comprises two or more antigenic epitopes selected from the group consisting of MOG₁₋₂₀, MBP₁₃₋₃₂, MOG₃₅₋₅₅, MBP₁₄₆₋₁₇₀, PLP₁₃₉₋₁₅₄, MBP₁₁₁₋₁₂₉, and MBP₈₃₋₉₉;
wherein said two or more antigenic epitopes are separated by a linker;
wherein said biodegradable particle has a diameter of about 200 nm to 1000 nm and;
wherein said biodegradable particle has a negative zeta potential of less than −30 mV.

112-115. (canceled)

116. A method of treating primary biliary cirrhosis in a subject comprising administering to the subject an effective amount of a biodegradable particle comprising one or more fusion proteins encapsulated therein;
wherein each one of said one or more fusion proteins comprises two or more antigenic epitopes from pyruvate dehydrogenase dihydrolipoamide acetyltransferase (PCD-E2);
wherein said two or more antigenic epitopes are separated by a linker; and;
wherein said biodegradable particle has a negative zeta potential between about −100 mV and about 0 mV.
